(19) United States
(12) Patent Application Publication
(10) Pub. No.: US 2023/0375537 A1
(43) Pub. Date: Nov. 23, 2023

(54) BASE MATERIAL FOR MANUFACTURING SENSOR FOR ANALYZING DETECTION OBJECT, SENSOR FOR ANALYZING DETECTION OBJECT, AND METHOD FOR ANALYZING DETECTION OBJECT

(71) Applicant: NATIONAL UNIVERSITY CORPORATION KOBE UNIVERSITY, Hyogo (JP)

(72) Inventors: Toshifumi TAKEUCHI, Hyogo (JP); Hirobumi SUNAYAMA, Hyogo (JP); Eri TAKANO, Hyogo (JP)

(21) Appl. No.: 18/246,300

(22) PCT Filed: Aug. 27, 2021
(86) PCT No.: PCT/JP2021/031621

§ 371 Date: Mar. 22, 2023

(30) Foreign Application Priority Data
Sep. 23, 2020 (JP) 2020-159008
Mar. 1, 2021 (WO) PCT/JP2021/007726

(51) Int. Cl. G01N 33/533; G01N 33/543; G01N 33/545

(52) U.S. Cl. CPC G01N 33/533; G01N 33/54386; G01N 33/545; G01N 2333/96455; G01N 2333/912; G01N 2600/00

(57) ABSTRACT

The present disclosure provides a biosensor having improved sensitivity. The present disclosure also provides a base material for manufacturing a sensor for analyzing a detection object, said base material comprising a molecularly imprinted polymer having: a molecularly imprinted recess in which a target of the detection object is received; and a group for binding a specific bondable molecule to the target, said group being provided inside the molecularly imprinted recess. The base material is characterized in that the group for binding the specific bondable molecule is a group suited for a small substance, and/or is characterized in that the molecularly imprinted recess has a shape or structure suited for a small substance.

### TECHNICAL FIELD

The present disclosure relates to the technique of sensitively detecting a detection subject on a base material. More specifically, the present disclosure relates to a base material for making a sensor for analyzing a detection subject and a manufacturing method thereof, a sensor for analyzing a detection subject and a manufacturing method thereof, and a method of analyzing a detection subject.

### BACKGROUND ART

Small Extracellular Vesicles (sEVs) such as exosome are one of the endoplasmic reticula released from a cell, which is lipid bilayer vesicle with a diameter of 20 to 200 nm. A small extracellular vesicle encapsulates a protein and a nucleic acid such as miRNA or mRNA therein, and has a protein on the surface thereof. Since a small extracellular vesicle is characterized by such a substance, it is considered that it is possible to presume what kind of cell a discharged cell is by analyzing the characteristic of the small extracellular vesicle. In addition, the presence of a small extracellular vesicle is confirmed in various body fluids, which can be relatively easily collected.

A small extracellular vesicle discharged from a cancer cell comprises a substance derived from a tumor. Therefore, it is expected that it is possible to diagnose cancer by analyzing a substance comprised in a small extracellular vesicle in a body fluid. Furthermore, since a small extracellular vesicle is actively discharged by a cell, it is expected that some sort of characteristic is exhibited even at an early stage of cancer.

### SUMMARY OF INVENTION

### Solution to Problem

When the present inventors intentionally introduced a small substance instead of an antibody in a biosensor, which comprises a polymer (molecularly imprinted polymer: MIP) having a hole that had been formed by molecular imprinting technique and is selectively introduced with an antibody in said hole, it was discovered that, regardless of the natural binding capacity (hereafter, “binding capacity” refers to affinity) of said small substance being low, the binding capacity per target would be increased to a surprising level which surpasses the case of introducing an antibody. The present disclosure was completed by carry out further examinations based on this finding.

The present disclosure comprises a base material for making a sensor for analyzing a detection subject and a manufacturing method thereof, a sensor for analyzing a detection subject and a manufacturing method thereof, and a method of analyzing a detection subject. For example, the present disclosure presents inventions in the following forms.

While an antibody used for a biosensor greatly contributes to the performance of the biosensor by its excellent specificity and affinity, the present disclosure provides improvement in the sensitivity thereof.

The present disclosure provides a biosensor with improved sensitivity.

### (Item 1)

A base material for use in making a sensor for use in analyzing a detection subject, comprising a molecularly imprinted polymer having:
a molecular imprint recessed part that receives a target of a detection subject; and
a group for use in binding a binding molecule to the target provided within the molecular imprint recessed part,
wherein the group for use in binding a binding molecule is a group adapted to a small substance.

### (Item 2)

A base material for use in making a sensor for use in analyzing a detection subject, comprising a molecularly imprinted polymer having:
a molecular imprint recessed part that receives a target of a detection subject; and
a group for use in binding a binding molecule to the target provided within the molecular imprint recessed part,
wherein the molecular imprint recessed part has a shape or configuration adapted to a small substance.

### (Item 3)

The base material for use in making a sensor for use in analyzing a detection subject of any one of the items above, further having a group for use in binding a signal substance within the molecular imprint recessed part.

### (Item 4)

The base material for use in making a sensor for use in analyzing a detection subject of any one of the items above, wherein the group for use in binding a binding molecule and the group for use in binding a signal substance are independent from one another in the molecular imprint recessed part surface.

### (Item 5)

The base material for use in making a sensor for use in analyzing a detection subject of any one of the items above, wherein the group for use in binding a binding molecule and the group for use in binding a signal substance configure the functional group shown in Formula (1) below within the molecular imprint recessed part.

[Chemical 1]

—R₂-L¹-R¹ (1)

<R¹ represents the group for use in binding a binding molecule, L¹ represents a direct binding or linking group, and R² represents a linking group comprising the group for use in binding a signal substance.>

### (Item 6)

The base material for use in making a sensor for use in analyzing a detection subject of any one of the items above, wherein a linking group comprising the group for use in binding a signal substance is represented by Formulas (2) and (3) below.

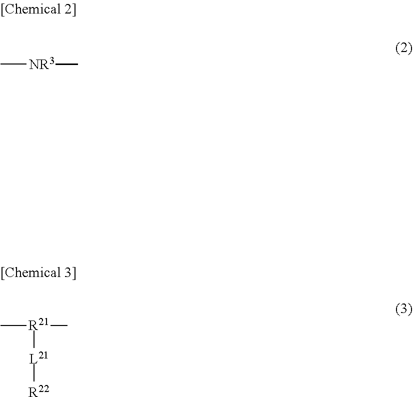

<R³ represents an alkyl group with a carbon number of 1 to 8 that may comprise hydrogen or a polyalkylene glycol chain, R²¹ represents a nitrogen atom or hydrocarbon, L²¹ represents a direct binding or linking group, and R²² represents the group for use in binding a signal substance.>

### (Item 7)

The base material for use in making a sensor for use in analyzing a detection subject of any one of the items above, wherein the group for use in binding a binding molecule is a group selected from the group consisting of an amino group, a carboxyl group, a boronic acid group, a cis-diol group, an aldehyde group, a ketone group, an oxyamino group, a biotin (biocytin, desthiobiotin) group, a nickel-nitrilotriacetic acid-driven group, a thiol group, and a pyridyl disulfide group.

### (Item 8)

The base material for use in making a sensor for use in analyzing a detection subject of any one of the items above, wherein the group for use in binding a binding molecule is a group selected from the group consisting of a nickel-nitrilotriacetic acid-driven group, a thiol group, and a pyridyl disulfide group.

### (Item 9)

The base material for use in making a sensor for use in analyzing a detection subject of any one of the items above, wherein the group for use in binding a binding molecule of Item 7 is adapted to a small substance via an Fc domain binding protein of an antibody, avidin, streptavidin, NeutrAvidin, or a polymer, dendrimer, or oligoDNA comprising one or more of the binding group of any one of the items above.

### (Item 10)

The base material for use in making a sensor for use in analyzing a detection subject of any one of the items above, wherein the Fc domain binding protein of an antibody is protein A, protein G, protein L, or protein A/G.

### (Item 11)

The base material for use in making a sensor for use in analyzing a detection subject of any one of the items above, having an interaction group for a mold of the molecular imprint recessed part or the detection subject, within the molecular imprint recessed part.

### (Item 12)

The base material for use in making a sensor for use in analyzing a detection subject of any one of the items above, wherein the binding molecule is a specific binding molecule.

### (Item 13)

The base material for use in making a sensor for use in analyzing a detection subject of any one of the items above, wherein the specific binding molecule is selected from the group consisting of an antibody mimetic, a phospholipid binding protein, an artificial (polymer) receptor, and lectin.

### (Item 14)

The base material for use in making a sensor for use in analyzing a detection subject of any one of the items above, wherein the detection subject is a microparticle having a film structure.

### (Item 15)

The base material for use in making a sensor for use in analyzing a detection subject of any one of the items above, wherein a mold of the molecular imprint recessed part is a particle core or a biomolecule.

### (Item 16)

The base material for use in making a sensor for use in analyzing a detection subject of any one of the items above, wherein a mold of the molecular imprint recessed part is a silica particle or serum protein.

### (Item 17)

The base material for use in making a sensor for use in analyzing a detection subject of any one of the items above, wherein a mold of the molecular imprint recessed part is a serum protein, the detection subject is a microparticle having a film structure, and the target is a molecule expressed on a surface of the microparticle.

### (Item 18)

The base material for use in making a sensor for use in analyzing a detection subject of any one of the items above, comprising a base plate and a polymer film provided on a surface of the base plate, wherein the imprint polymer configures the polymer.

### (Item 19)

The base material for use in making a sensor for use in analyzing a detection subject of any one of the items above, wherein the imprint polymer is particle-like.

### (Item 20)

The base plate for use in making a sensor for use in analyzing a detection subject of any one of the items above, wherein the imprint polymer that is particle-like is immobilized on a base plate.

### (Item 21)

A sensor for use in analyzing a detection subject, comprising:

the base material for use in making a sensor for use in analyzing a detection subject of any one of the items above; and a specific binding group for a target of the detection subject bound to the group for use in binding a specific binding molecule, wherein the specific binding molecule is a small substance group.

### (Item 22)

The sensor for use in analyzing a detection subject of any one of the items above, further comprising a signal group bound to the group for use in binding a signal substance.

### (Item 23)

An analysis method of a detection subject, comprising: the step of contacting a sample comprising a detection subject to the sensor for use in analyzing a detection subject of any one of the items above and binding the detection subject to the specific binding group; and the step of detecting a bond between the specific binding group and the detection subject.

### (Item 24)

A method of manufacturing a base material for use in making a sensor for use in analyzing a detection subject, comprising:
the monolayer formation step (1-1) of forming a monolayer having a group for use in binding a binding molecule to a target of a detection subject and a polymerization initiating group on a surface at a base plate;
the step (1-2) of introducing a mold to the group for use in binding a binding molecule via a first reversible linking group;
the step (1-3) of adding a polymerizable monomer, using the polymerizable monomer as a substrate, using the polymerization initiating group as a polymerizable initiating agent, and synthesizing a molecularly imprinted polymer for a part of a surface of the mold to form a polymer film on the base plate surface; and
the step (1-4) of cleaving the first reversible linking group for conversion to the group for use in binding a binding molecule as well as removal of the mold,
wherein the group for use in binding a binding molecule is a group adapted to a small substance.

### (Item 25)

A method of manufacturing a base material for use in making a sensor for use in analyzing a detection subject, comprising:
the monolayer formation step (1-1) of forming a monolayer having a group for use in binding a binding molecule to a target of a detection subject and a polymerization initiating group on a surface at a base plate;
the step (1-2) of introducing a mold to the group for use in binding a binding molecule via a first reversible linking group;
the step (1-3) of adding a polymerizable monomer, using the polymerizable monomer as a substrate, using the polymerization initiating group as a polymerizable initiating agent, and synthesizing a molecularly imprinted polymer for a part of a surface of the mold to form a polymer film on the base plate surface; and
the step (1-4) of cleaving the first reversible linking group for conversion to the group for use in binding a binding molecule as well as removal of the mold,
wherein the molecular imprint recessed part has a shape or configuration adapted to a small substance.

### (Item 26)

A method of manufacturing the base material for use in making a sensor for use in analyzing a detection subject of any one of the items above, comprising:
the modification step of modifying the surface of the mold via a polymerizable functional group via a second reversible linking group,
wherein the polymerizable monomer and the polymerizable functional group are used as a substrate and the polymerization initiating group is used as a polymerizable initiating agent to synthesize a molecularly imprinted polymer for a part of the surface of the mold in the step (1-3), and
wherein the first reversible linking group and the second reversible linking group are cleaved for conversion to the group for use in binding a binding molecule and a group for use in binding a signal substance, respectively, as well as removal of the mold in the step (1-4).

### (Item 27)

A method of manufacturing a base material for use in making a sensor for use in analyzing a detection subject, comprising:
the step (1-11) of preparing a mold of a molecular imprint wherein a surface is modified with a polymerizable functional group via a first reversible linking group;
the step (1-12) of using the polymerizable functional group as a substrate and synthesizing a molecularly imprinted polymer for a part of the surface of the mold; and
the step (1-13) of cleaving the first reversible linking group and removing the mold from the molecularly imprinted polymer to generate the group for use in binding a binding molecule,
wherein the group for use in binding a binding molecule is adapted to a small substance.

### (Item 28)

A method of manufacturing a base material for use in making a sensor for use in analyzing a detection subject, comprising:
the step (1-11) of preparing a mold of a molecular imprint wherein a surface is modified with a polymerizable functional group via a first reversible linking group;
the step (1-12) of using the polymerizable functional group as a substrate and synthesizing a molecularly imprinted polymer for a part of the surface of the mold; and
the step (1-13) of cleaving the first reversible linking group and removing the mold from the molecularly imprinted polymer to generate the group for use in binding a binding molecule,
wherein the molecular imprint recessed part has a shape or configuration adapted to a small substance.

### (Item 29)

A method of manufacturing a base material for use in making a sensor for use in analyzing a detection subject, comprising:
the step (1-21) of preparing a mold of a molecular imprint wherein a surface is modified with a polymerizable functional group via a third reversible linking group;
the step (1-22) of using the polymerizable functional group as a substrate and synthesizing a molecularly imprinted polymer for a part of the surface of the mold;
the step (1-23) of cleaving the third reversible linking group and removing the mold from the molecularly imprinted polymer; and
the step (1-24) of introducing a molecule having functional group comprising a group for use in binding a binding molecule for a target of a detection subject and a group for use in binding a signal substance via third reversible linking group,
wherein the group for use in binding a binding molecule is a group adapted to a small substance.

### (Item 30)

A method of manufacturing a base material for use in making a sensor for use in analyzing a detection subject, comprising:
the step (1-21) of preparing a mold of a molecular imprint wherein a surface is modified with a polymerizable functional group via a third reversible linking group;
the step (1-22) of using the polymerizable functional group as a substrate and synthesizing a molecularly imprinted polymer for a part of the surface of the mold;
the step (1-23) of cleaving the third reversible linking group and removing the mold from the molecularly imprinted polymer; and
the step (1-24) of introducing a molecule having functional group comprising a group for use in binding a binding molecule for a target of a detection subject and a group for use in binding a signal substance via third reversible linking group,
wherein the molecular imprint recessed part has a shape or configuration adapted to a small substance.

### (Item 31)

The method of manufacturing a base material for use in making a sensor for use in analyzing a detection subject of any one of the items above, further using a monomer comprising an interaction group for the mold as the substrate in the step (1-22).

### (Item 32)

A method of manufacturing the base material for use in making a sensor for use in analyzing a detection subject of any one of the items above, wherein the binding molecule is a specific binding molecule.

### (Item 33)

A method of manufacturing a sensor for use in analyzing a detection subject, comprising:
the step (1) of carrying out the method of manufacturing a base material for use in making a sensor for use in analyzing a detection subject of any one of the items above; and
the step (2) of binding the binding molecule to the group for use in binding a binding molecule.

### (Item 34)

A method of manufacturing a sensor for use in analyzing a detection subject, comprising:
the step (1) of carrying out the method of manufacturing a base material for use in making a sensor for use in analyzing a detection subject of any one of the items above;
the step (2) of binding the binding molecule to the group for use in binding a binding molecule; and
the step (3) of binding a signal substance to the group for use in binding a signal substance.

### (Item 35)

A method of diagnosing and examining a subject using the sensor for analysis of one of the items above, the diagnosis method comprising:
A) the step of collecting a sample (comprising, for example, urine, cerebrospinal fluid, sweat, nasal mucus, saliva, blood, or tear) of a subject;
B) the step of contacting the sample with the sensor for analysis;
C) the step of detecting a signal from the sensor for analysis; and
D) the step of analyzing the signal and then diagnosing or examining the subject (e.g., determining presence/absence or progression of a disease).

### (Item 36)

A program that causes a computer to execute a method of diagnosing and examining a subject using a diagnosis or examination system comprising the sensor for analysis of any one of the items above, the method comprising:
A) the step of providing a sample (comprising, for example, urine, cerebrospinal fluid, sweat, nasal mucus, saliva, blood, or tear) obtained from the subject to the diagnosis or examination system;
B) the step of activating the diagnosis or examination system to contact the sample with the sensor for analysis;
C) the step of the diagnosis or examination system analyzing a signal from the sensor for analysis; and
D) the step of the diagnosis or examination system analyzing the signal and then diagnosing or detecting the subject (determining presence/absence or progression of a disease from a detection result).

### (Item 37)

A record medium storing a program that causes a computer to execute a method of diagnosing or examining a subject using the sensor for analysis of any one of the items above, the method comprising:
A) the step of providing a sample (comprising, for example, urine, cerebrospinal fluid, sweat, nasal mucus, saliva, blood, or tear) obtained from the subject to the diagnosis or examination system;
B) the step of activating the diagnosis or examination system to contact the sample with the sensor for analysis;
C) the step of the diagnosis or examination system analyzing a signal from the sensor for analysis; and
D) the step of the diagnosis or examination system analyzing the signal and then diagnosing or detecting the subject (determining presence/absence or progression of a disease from a detection result).

### (Item 38)

A diagnosis or examination system, the system comprising:
A) a providing unit that provides a sample (comprising, for example, urine, cerebrospinal fluid, sweat, nasal mucus, saliva, blood, or tear) obtained from the subject;
B) an activation unit that activates so as to contact the sample to the sensor for analysis;
C) a detection unit that detects a signal from the sensor for analysis; and
D) a diagnosis unit that analyzes the signal and then provides diagnosis to the subject (determines presence/absence or progression of disease from a detection result).

### Item. 1

A base material for use in making a sensor for use in analyzing a detection subject, comprising a molecularly imprinted polymer having:
a molecular imprint recessed part that receives a target molecule of a detection subject; and
a group for use in binding a specific binding molecule to the target molecule provided within the molecular imprint recessed part,
wherein the group for use in binding a specific binding molecule is a specific binding protein or peptide with a molecular weight of 45,000 or less.

### Item. 2

The base material for use in making a sensor for use in analyzing a detection subject of item 1, further having a group for use in binding a signal substance within the molecular imprint recessed part.

### Item. 3

The base material for use in making a sensor for use in analyzing a detection subject of item 2, wherein the group for use in binding a specific binding molecule and the group for use in binding a signal substance are independent from one another in the molecular imprint recessed part surface.

### Item. 4

The base material for use in making a sensor for use in analyzing a detection subject of item 2, wherein the group for use in binding a specific binding molecule and the group for use in binding a signal substance configure the functional group shown in Formula (1) below within the molecular imprint recessed part.

[Chemical 4]

—R²-L¹-R¹ (1)

<R¹ represents the group for use in binding a specific binding molecule, L¹ represents a direct binding or linking group, and R² represents a linking group comprising the group for use in binding a signal substance.>

### Item. 5

The base material for use in making a sensor for use in analyzing a detection subject of item 4, wherein a linking group comprising the group for use in binding a signal substance is represented by Formulas (2) and (3) below.

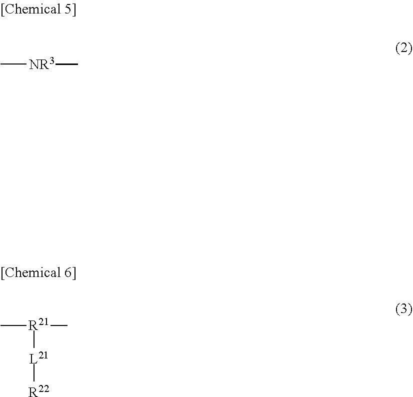

<R³ represents an alkyl group with a carbon number of 1 to 8 that may comprise hydrogen or a polyalkylene glycol chain, R²¹ represents a nitrogen atom or hydrocarbon, L²¹ represents a direct binding or linking group, and R²² represents the group for use in binding a signal substance.>

### Item. 6

The base material for use in making a sensor for use in analyzing a detection subject of any of items 1 to 5, having an interaction group for a mold of the molecular imprint recessed part or the detection subject within the molecular imprint recessed part.

### Item. 7

The base material for use in making a sensor for use in analyzing a detection subject of any of items 1 to 6, wherein the specific binding molecule is selected from the group consisting of a nanobody, an antibody mimetic, a phospholipid binding protein, and lectin.

### Item. 8

The base material for use in making a sensor for use in analyzing a detection subject of any of items 1 to 7, wherein the detection subject is a microparticle having a film structure.

### Item. 9

The base material for use in making a sensor for use in analyzing a detection subject of any of items 1 to 8, wherein a mold of the molecular imprint recessed part is a plasma protein, the detection subject is a microparticle having a film structure, and the target molecule is a molecule expressed on the microparticle.

### Item. 10

The base material for use in making a sensor for use in analyzing a detection subject of any of items 1 to 9, comprising a base plate and a polymer film provided on a surface of the base plate, wherein the imprint polymer configures the polymer.

### Item. 11

The base material for use in making a sensor for use in analyzing a detection subject of any of items 1 to 9, wherein the imprint polymer is particle-like.

### Item. 12

The base plate for use in making a sensor for use in analyzing a detection subject of item 11, wherein the imprint polymer that is particle-like is immobilized on a base plate.

### Item. 13

A sensor for use in analyzing a detection subject, comprising:
the base material for use in making a sensor for use in analyzing a detection subject of any one of items 1 to 12; and
a specific binding group for a target molecule of the detection subject bound to the group for use in binding a specific binding molecule,
wherein the specific binding molecule is a specific binding protein or peptide with a molecular weight of 45,000 or less.

### Item. 14

The sensor for use in analyzing a detection subject of item 13, further comprising a signal group bound to the group for use in binding a signal substance.

### Item. 15

An analysis method of a detection subject, comprising:
the step of contacting a sample comprising a detection subject to the sensor for use in analyzing a detection subject of item 13 or 14 and binding the detection subject to the specific binding group; and
the step of detecting a bond between the specific binding group and the detection subject.

### Item. 16

A method of manufacturing a base material for use in making a sensor for use in analyzing a detection subject, comprising:
the monolayer formation step (1-1) of forming a monolayer having a group for use in binding a specific binding molecule to a target of a detection subject and a polymerization initiating group on a surface at a base plate;
the step (1-2) of introducing a mold to the group for use in binding a specific binding molecule via a first reversible linking group;
the step (1-3) of adding a polymerizable monomer, using the polymerizable monomer as a substrate, using the polymerization initiating group as a polymerizable initiating agent, and synthesizing a molecularly imprinted polymer for a part of a surface of the mold to form a polymer film on the base plate surface; and
the step (1-4) of cleaving the first reversible linking group for conversion to the group for use in binding a specific binding molecule as well as removal of the mold,
wherein the specific binding molecule is a specific binding protein or peptide with a molecular weight of 45,000 or less.

### Item. 17

A method of manufacturing the base material for use in making a sensor for use in analyzing a detection subject of item 16, comprising:
the modification step of modifying the surface of the mold via a polymerizable functional group via a second reversible linking group,
wherein the polymerizable monomer and the polymerizable functional group are used as a substrate and the polymerization initiating group is used as a polymerizable initiating agent to synthesize a molecularly imprinted polymer for a part of the surface of the mold in the step (1-3), and
wherein the first reversible linking group and the second reversible linking group are cleaved for conversion to the group for use in binding a specific binding molecule and a group for use in binding a signal substance, respectively, as well as removal of the mold in the step (1-4).

### Item. 18

A method of manufacturing a base material for use in making a sensor for use in analyzing a detection subject, comprising:
the step (1-11) of preparing a mold of a molecular imprint wherein a surface is modified with a polymerizable functional group via a first reversible linking group;
the step (1-12) of using the polymerizable functional group as a substrate and synthesizing a molecularly imprinted polymer for a part of the surface of the mold; and
the step (1-13) of cleaving the first reversible linking group and removing the mold from the molecularly imprinted polymer to generate the group for use in binding a specific binding molecule,
wherein the specific binding molecule is a specific binding protein or peptide with a molecular weight of 45,000 or less.

### Item. 19

A manufacturing method a base material for use in making a sensor for use in analyzing a detection subject, comprising:
the step (1-21) of preparing a mold of a molecular imprint wherein a surface is modified with a polymerizable functional group via a third reversible linking group;
the step (1-22) of using the polymerizable functional group as a substrate and synthesizing a molecularly imprinted polymer for a part of the surface of the mold;
the step (1-23) of cleaving the third reversible linking group and removing the mold from the molecularly imprinted polymer; and
the step (1-24) of introducing a molecule having functional group comprising a group for use in binding a specific binding molecule for a target molecule of a detection subject and a group for use in binding a signal substance via third reversible linking group,
wherein the specific binding molecule is a specific binding protein or peptide with a molecular weight of 45,000 or less.

### Item. 20

The method of manufacturing a base material for use in making a sensor for use in analyzing a detection subject of item 19, further using a monomer comprising an interaction group for the mold as the substrate in the step (1-22).

### Item. 21

A method of manufacturing a sensor for use in analyzing a detection subject, comprising:
the step (1) of carrying out the method of manufacturing a base material for use in making a sensor for use in analyzing a detection subject of any of items 16 to 20; and
the step (2) of binding the specific binding molecule to the group for use in binding a specific binding molecule.

### Item. 22

A method of manufacturing a sensor for use in analyzing a detection subject, comprising:
the step (1) of carrying out the method of manufacturing a base material for use in making a sensor for use in analyzing a detection subject of any of items 17, 19, and 20;
the step (2) of binding the specific binding molecule to the group for use in binding a specific binding molecule; and
the step (3) of binding a signal substance to the group for use in binding a signal substance.

### Advantageous Effects of Invention

The present disclosure provides a biosensor with improved sensitivity regardless of the natural binding capacity of an introduced specific binding molecule being low (relatively great dissociation constant Kd). Although the specific mechanism of achieving such excellent effect is not certain, it is considered that, due to the fact that the introduced specific binding molecule is a relatively small molecule, it becomes possible to distribute a plurality of specific binding molecules in a fine hole formed by molecular imprinting technique, whereby a detection subject is captured with a plurality of bonds upon sensing, raising the binding capacity of the specific binding molecule per detection subject to a surprising level (significantly reduced dissociation constant Kd).

### BRIEF DESCRIPTION OF DRAWINGS

FIG. 1A is a schematic diagram of an example of the base material for making an analysis sensor of a detection subject of the present disclosure.

FIG. 1B-1 is a schematic diagram of an example of the analysis sensor of a detection subject of the present disclosure corresponding to FIG. 1A.

FIG. 1B-2 is a schematic diagram of another example of the analysis sensor of a detection subject of the present disclosure corresponding to FIG. 1A.

FIG. 2A is a schematic diagram of an example of the base material for making an analysis sensor of a detection subject of the present disclosure.

FIG. 2B-1 is a schematic diagram of an example of the analysis sensor of a detection subject of the present disclosure corresponding to FIG. 2A.

FIG. 2B-2 is a schematic diagram of another example of the analysis sensor of a detection subject of the present disclosure corresponding to FIG. 2A.

FIG. 3A is a schematic diagram of an example of the base material for making an analysis sensor of a detection subject of the present disclosure.

FIG. 3B is a schematic diagram of the analysis sensor of a detection subject of the present disclosure corresponding to FIG. 3A.

FIG. 4A is a schematic diagram of an example of the base material for making an analysis sensor of a detection subject of the present disclosure.

FIG. 4B is a schematic diagram of the analysis sensor of a detection subject of the present disclosure corresponding to FIG. 4A.

FIG. 5 is (A) a schematic diagram of the case wherein an example of the base material for making an analysis sensor of a detection subject of the present disclosure of FIG. 1A is like a film provided on a base plate, and a schematic diagram of the analysis sensor of a detection subject of the present disclosure corresponding thereto.

FIG. 6 is (A) a schematic diagram of the case wherein an example of the base material for making an analysis sensor of a detection subject of the present disclosure of FIG. 1A is a particle, and (B) a schematic diagram of the analysis sensor of a detection subject of the present disclosure corresponding thereto.

FIG. 7 is (A) a schematic diagram of the case wherein an example of the base material for making an analysis sensor of a detection subject of the present disclosure of FIG. 1A is a molecule immobilized on a base plate, and (B) a schematic diagram of the analysis sensor of a detection subject of the present disclosure corresponding thereto.

FIG. 8 is (A) a schematic diagram of the case wherein an example of the base material for making an analysis sensor of a detection subject of the present disclosure of FIG. 2A is like a film provided on a base plate, and (B1), (B2) a schematic diagram of the analysis sensor of a detection subject of the present disclosure corresponding thereto.

FIG. 9 is (A) a schematic diagram of the case wherein an example of the base material for making an analysis sensor of a detection subject of the present disclosure of FIG. 2A is a particle, and (B) a schematic diagram of the analysis sensor of a detection subject of the present disclosure corresponding thereto.

FIG. 10 is (A) a schematic diagram of the case wherein an example of the base material for making an analysis sensor of a detection subject of the present disclosure of FIG. 4A is a particle, and (B) a schematic diagram of the analysis sensor of a detection subject of the present disclosure corresponding thereto.

FIG. 11 is (A) a schematic diagram of the case wherein an example of the base material for making an analysis sensor of a detection subject of the present disclosure of FIG. 3A is a particle immobilized on a base plate, and (B) a schematic diagram of the analysis sensor of a detection subject of the present disclosure corresponding thereto.

FIG. 12-la schematically shows an embodiment of preparing a mold in a manufacturing method of the base material 10a-C′ for making a sensor for analyzing a detection subject that is base plate-type which is shown in FIG. 8(A).

FIG. 12-1b schematically shows another embodiment of preparing a bold in a manufacturing method of the base material 10a-C′ for making a sensor for analyzing a detection subject that is base plate-type which is shown in FIG. 8(A).

FIG. 12-2 schematically shows the step (1-1) in a manufacturing method of the base material 10a-C′ for making a sensor for analyzing a detection subject that is base plate-type which is shown in FIG. 8(A).

FIG. 12-3a schematically shows the step (1-2) (the case of using the mold 601 obtained in FIG. 12-1a) in a manufacturing method of the base material 10a-C′ for making a sensor for analyzing a detection subject that is base plate-type which is shown in FIG. 8(A).

FIG. 12-3b schematically shows the step (1-2) (the case of using the mold 602 obtained in FIG. 12-1b) in a manufacturing method of the base material 10a-C′ for making a sensor for analyzing a detection subject that is base plate-type which is shown in FIG. 8(A).

FIG. 12-4 schematically shows the step (1-3) in a manufacturing method of the base material 10a-C′ for making a sensor for analyzing a detection subject that is base plate-type which is shown in FIG. 8(A).

FIG. 12-5 shows the step (1-4) in a manufacturing method of the base material 10a-C′ for making a sensor for analyzing a detection subject that is base plate-type which is shown in FIG. 8(A).

FIG. 12-6 schematically shows a manufacturing method of the sensor 10a-C for analyzing a detection subject that is base plate-type which is shown in FIG. 8(B).

FIG. 13-1a schematically shows the step (1-11) (the case of using the mold 601) in a manufacturing method of the base material 10-P′ for making a sensor for analyzing a detection subject that is particle-like which is shown in FIG. 6(A).

FIG. 13-1b schematically shows the step (1-11) (the case of using the mold 602) in a manufacturing method of the base material 10-P′ for making a sensor for analyzing a detection subject that is particle-like which is shown in FIG. 6(A).

FIG. 13-2 schematically shows the step (1-12) in a manufacturing method of the base material 10-P′ for making a sensor for analyzing a detection subject that is particle-like which is shown in FIG. 6(A).

FIG. 13-3 schematically shows an example of the step (1-13) in a manufacturing method of the base material 10-P′ for making a sensor for analyzing a detection subject that is particle-like which is shown in FIG. 6(A).

FIG. 13-4 schematically shows the manufacturing method of the sensor 10-P for analyzing a detection subject that is particle-like which is shown in FIG. 6(B).

FIG. 14-1 schematically shows the step (1-21) in a manufacturing method of the base material 10c-P′ for making a sensor for analyzing a detection subject that is particle-like which is shown in FIG. 10(A).

FIG. 14-2 schematically shows the step (1-22) in a manufacturing method of the base material 10c-P′ for making a sensor for analyzing a detection subject that is particle-like which is shown in FIG. 10(A).

FIG. 14-3 schematically shows the step (1-23) in a manufacturing method of the base material 10c-P′ for making a sensor for analyzing a detection subject that is particle-like which is shown in FIG. 10(A).

FIG. 14-4 schematically shows the step (1-24) in a manufacturing method of the base material 10c-P′ for making a sensor for analyzing a detection subject that is particle-like which is shown in FIG. 10(A).

FIG. 14-5 schematically shows the manufacturing method of the sensor 10c-P for analyzing a detection subject that is particle-like which is shown in FIG. 10(B).

FIG. 15 schematically shows the step of fixing a sensor base material for analyzing a detection subject that is particle-like to a base plate during FIG. 14-3 and FIG. 14-4.

FIG. 16-1 schematically shows an example (in an environment of high concentration of a mold (60c) of a molecular imprint recessed part 21c) of double targeting using the sensor 10c-P for analyzing a detection subject that is particle-like which is shown in FIG. 10(B).

FIG. 16-2 schematically shows an example (in an environment of low concentration of a mold (60c) of a molecular imprint recessed part 21c) of double targeting using the sensor 10c-P for analyzing a detection subject that is particle-like which is shown in FIG. 10(B).

FIG. 17A shows the relationship between a relative fluorescence intensity and an exosome concentration, using the analysis sensor 10a-C (introducing Alexa Fluor 647 as a signal substance) of a detection subject that is base plate-type of Example 1.

FIG. 17B shows the relationship between a relative fluorescence intensity and an exosome concentration, using the analysis sensor that is not introduced with an affibody of Comparative Example 1.

FIG. 18 shows the relationship between a relative fluorescence intensity and an exosome concentration, using the analysis sensor 10a-C (introducing rhodamine as a signal substance) of a detection subject (exosome) that is base plate-type of Example 2.

FIG. 19 shows the relationship between a relative fluorescence intensity and an exosome concentration, using the analysis sensor of a detection subject (exosome) that is base plate-type of Example 3 (provided that the sensor is a type of analysis sensor 101 product of a detection subject that is base plate-type utilizing a signal base plate fused to a specific binding group).

FIG. 20 shows a TEM image of a particle-like molecularly imprinted polymer of the analysis sensor 10-PC of a detection subject that is particle-like and immobilized on a base plate of Example 4.

FIG. 21 shows the relationship between an amount of change of response and exosome solution concentration, using the analysis sensor 10-PC of a detection subject (exosome) that is particle-like and immobilized on a base plate of Example 4.

FIG. 22 shows the relationship between a relative fluorescence intensity and an exosome concentration, using the analysis sensor 10b-PC of a detection subject (exosome) that is particle-like and immobilized on a base plate of Example 5.

FIG. 23 shows relative fluorescence intensity change of the case of adding exosome derived from an HER2 overexpression-type breast cancer cell line regarding the analysis sensor 10b-PC of a detection subject (exosome) that is particle-like and immobilized on a base plate of Example 5 and a sensor for analysis wherein an anti-HSA affibody is introduced instead of an anti-HER2 affibody.

FIG. 24 shows a TEM image of a particle-like molecularly imprinted polymer of the analysis sensor 10c-P of a detection subject that is particle-like and immobilized on a base plate of Example 6.

FIG. 25 shows double targeting in solutions with different concentration ratio of exosome and HSA, using the analysis sensor 10c-P of a detection subject (exosome) that is particle-like and immobilized on a base plate of Example 6.

### DESCRIPTION OF EMBODIMENTS

The present disclosure is explained below in further detail.

Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

The terms and general technique used in the present disclosure are appropriately explained herein.

### <1. A Base Material for Making a Sensor for Analyzing a Detection Subject>

The base material for making a sensor for analyzing a detection subject of the present disclosure is a base material that becomes a material for making a sensor for analysis of the invention discussed below. This base material for making a sensor for analysis is configured to enable a user to easily carry out customization to a sensor capable of even more sensitive detection of a detection subject.

### <1-1. Basic Configuration (Sensing Precursor Part)>

The base material for making an analysis sensor of a detection subject of the present disclosure comprises a molecularly imprinted polymer having a molecular imprint recessed part that receives a target of a detection subject and a group for binding a specific binding molecule to said target, which is provided within said molecular imprint recessed part; characterized in that said group for binding the specific binding molecule is a group adapted to a small substance, and/or characterized in that the molecular imprint recessed part has a shape or configuration adapted to a small substance. This basic configuration configures a sensing precursor part (i.e., a portion configuring a sensing part when customized to a sensor for analysis) of a base material for making an analysis sensor of a detection subject.

As used herein, “target” refers to a targeted molecule or substance. For example, a target may be a molecule or a molecular aggregate.

As used herein, “small substance” refers to any substance smaller than an antibody, which can be interchangeable with “small binding molecular substance”. Since the criterion of the molecular weight is about 150,000, a molecular weight that is less than said number (i.e., less than 150,000) may be included in this concept. Preferable molecular weight includes about 140,000 or less, about 130,000 or less, about 120,000 or less, about 110,000 or less, about 100,000 or less, about 90,000 or less, about 80,000 or less, about 70,000 or less, about 70,000 or less, about 60,000 or less, about 50,000 or less, about 40,000 or less, about 30,000 or less, about 22,000 or less, about 20,000 or less, about 19,000 or less, about 18,000 or less, about 17,000 or less, about 16,000 or less, about 15,000 or less, about 14,000 or less, about 13,000 or less, about 12,000 or less, about 11,000 or less, about 10,000 or less, about 9,000 or less, about 8,000 or less, about 7,000 or less, about 6,000 or less, and the like. The lower limit of the molecular weight of a small molecule can include, about 200 or more, about 300 or more, about 400 or more, about 500 or more, and about 600 or more.

Any type of substance can be used as a small substance, which can typically include protein, polypeptide, peptide, sugar, lipid, nucleic acid, nucleic acid derivative, artificial (polymer) receptor, other organic molecules, complex molecules thereof, and the like, preferably includes protein, polypeptide, and peptide, more preferably protein or peptide.

As used herein, “artificial (polymer) receptor” refers to a molecularly imprinted polymer nanoparticle (particle size of about 10 to 30 nm) that recognizes a target, which is used for a sandwich assay or the like instead of an antibody. Evan M. Peck and Bradley D. Smith, Chapter 1: Applications of Synthetic Receptors for Biomolecules, in Synthetic Receptors for Biomolecules: Design Principles and Applications, 2015, pp. 1-38 can be referred to as such artificial receptor.

As used herein, “group for binding” or “molecular imprint recessed part” being “adapted to . . . ” refers to the concept of being a configuration that is appropriately detected upon carrying out a detection process that sets a substance that is subjected to adaptation as a subject when used in a manufacturing base material for a sensor or a sensor wherein the “base for binding” or “molecular imprint recessed part” is comprised, regarding a substance that is subjected to adaptation (e.g., small substance, a molecule with the molecular weight of 45,000, or the like).

Typically, for example, “group for binding that is adapted to a small substance” is a group for binding that is appropriate for a small substance smaller than an antibody, wherein groups such as amino group, carboxy group, boronic acid group, cis-diol group, aldehyde group, ketone group, oxyamino group, hydrazide group, biotin (biocytin, desthiobiotin) group, nickel-nitrilotriacetic acid-driven group, thiol group, pyridyl disulfide group, metal complex, and the like may be preferably utilized. In addition, said group for binding may be adapted to a small substance via Fc domain binding proteins of an antibody such as protein A, protein G, protein L, and protein A/G, proteins such as avidin, streptavidin, and neutravidin, polymers comprising one or more of groups such as amino group, carboxy group, boronic acid group, cis-diol group, aldehyde group, ketone group, oxyamino group, hydrazide group, biotin (biocytin, desthiobiotin) group, nickel-nitrilotriacetic acid-driven group, thiol group, pyridyl disulfide group, and metal complex, dendrimer, oligo DNA, and the like.

Typically, for example, “a molecular imprint recessed part has a shape or configuration adapted to a small substance” refers to the concept of having a shape or configuration appropriate for detection when the molecular imprint recessed part is disposed in a manufacturing base material for a sensor or a sensor.

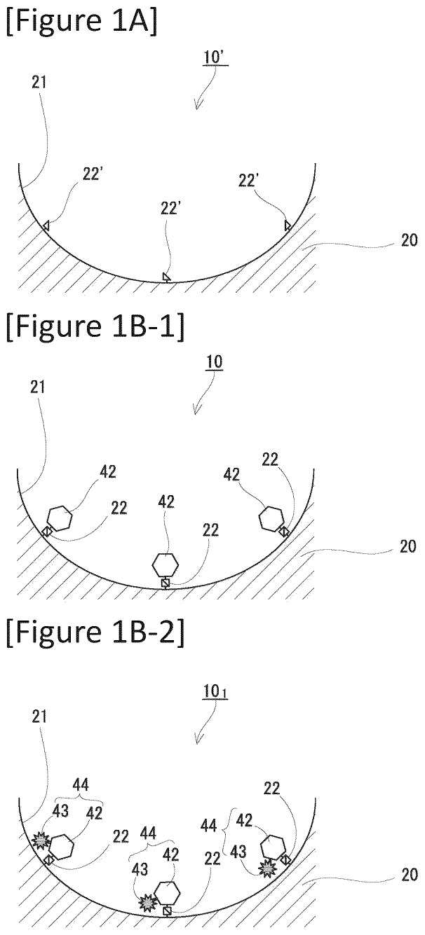

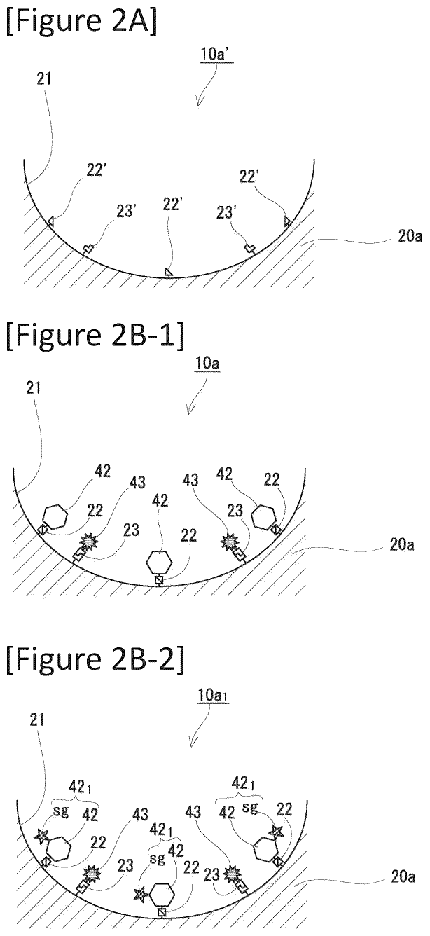

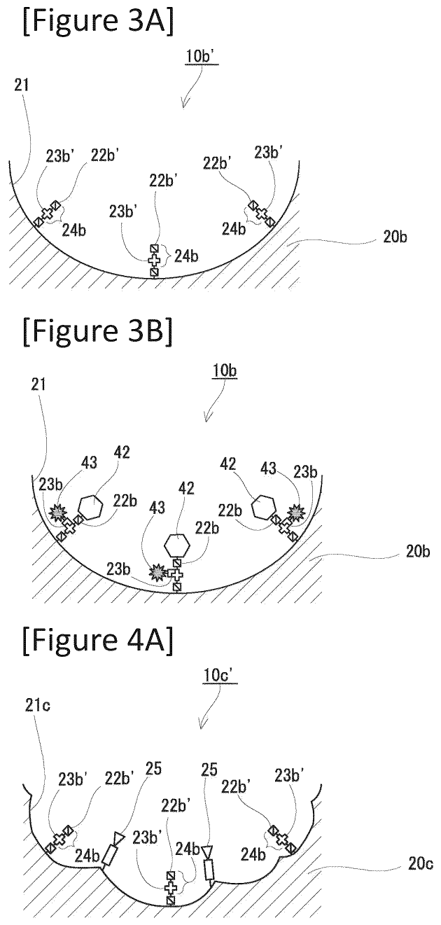

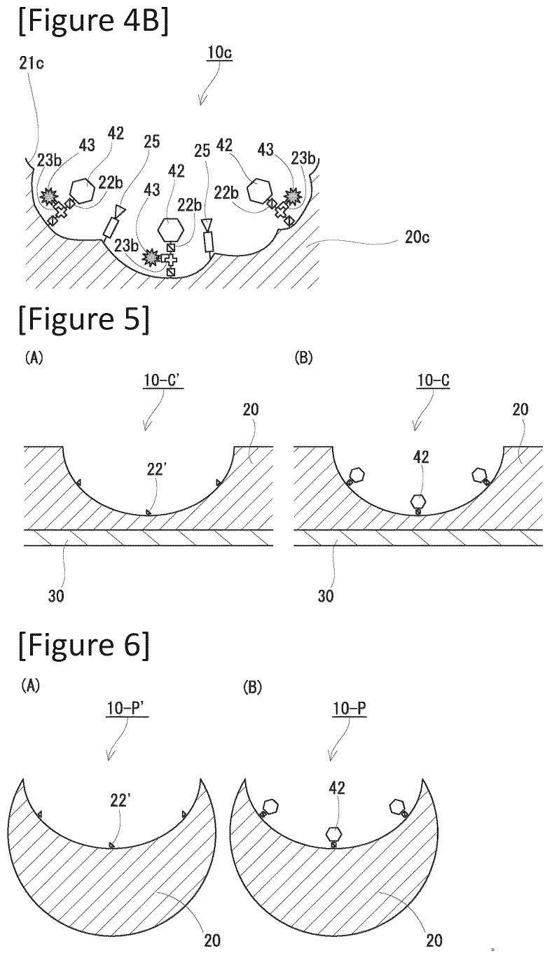

Some examples of the base material for making an analysis sensor of a detection subject of the present disclosure are schematically shown as base materials 10′, 10a′, 10b′, and 10c′ for making an analysis sensor of a detection subject in FIG. 1A, FIG. 2A, FIG. 3A, and FIG. 4A, respectively. The base materials for making an analysis sensor of a detection subject of the present disclosure shown in these diagrams are used to make sensors 10, 101, 10a, 10a′, 10b, and 10c for analyzing a detection subject schematically shown in FIG. 1B-1, FIG. 1B-2, FIG. 2B-1, FIG. 2-2, FIG. 3B, and FIG. 4B, respectively.

The base material 10′ for making an analysis sensor of a detection subject shown in FIG. 1A comprises a molecularly imprinted polymer 20 having a molecular imprint recessed part 21 that receives a target of a detection subject, and a group 22′ for binding a specific binding molecule to said target, which is provided within said molecular imprint recessed part 21.

The base material 10a′ for making an analysis sensor of a detection subject shown in FIG. 2A comprises a molecularly imprinted polymer 20a having a molecular imprint recessed part 21 that receives a target of a detection subject and a group 22′ for binding a specific binding molecule to said target and a group 23′ for signal substance binding, which are provided within the molecular imprint recessed part 21. In other words, the base material 10a′ for making an analysis sensor of a detection subject is the same as the base material 10′ for making an analysis sensor of a detection subject of FIG. 1A excluding the point that the group 23′ for signal substance binding is further provided within said molecular imprint recessed part 21. Furthermore, in the base material 10a′ for making an analysis sensor of a detection subject, in a molecular imprint recessed part 21 surface, the group 22′ for binding a specific binding molecule to a target and the group 23′ for binding a signal substance are independent from one another (i.e., spaced apart from one another in the molecular imprint recessed part 21 surface direction).

The base material 10b′ for making an analysis sensor of a detection subject shown in FIG. 3A comprises a molecularly imprinted polymer 20b having a molecular imprint recessed part 21 that receives a target of a detection subject, and a group 22b′ for binding a specific binding molecule to said target and a group 23b′ for signal substance binding, which are provided within said molecular imprint recessed part 21. In other words, the base material 10b′ for making an analysis sensor of a detection subject is the same as the base material 10a′ for making an analysis sensor of a detection subject of FIG. 2A in that the group 23b′ for signal substance binding is further provided within the molecular imprint recessed part 21. However, unlike the base material 10a′ for making an analysis sensor of a detection subject of FIG. 2A, in the base material 10b′ for making an analysis sensor of a detection subject, the group 22b′ for binding a specific binding molecule to a target and the group 23b′ for binding a signal substance configure a predetermined functional group 24b within the molecular imprint recessed part 21.

The base material 10c′ for making an analysis sensor of a detection subject shown in FIG. 4A comprises a molecularly imprinted polymer 20c having a molecular imprint recessed part 21c that receives a target of a detection subject, a group 22b′ for binding a specific binding molecule to a target and a group 23b′ for binding a signal substance, which are provided within said molecular imprint recessed part 21c, and an interaction group 25 for a mold of the molecular imprint recessed part 21c. In other words, the base material 10c′ for making an analysis sensor of a detection subject is the same as the base material 10b′ for making an analysis sensor of a detection subject of FIG. 3A in that the group 23b′ for signal substance binding is further comprised, and in that the group 22b′ for binding a specific binding molecule to a target and the group 23b′ for binding a signal substance configure a predetermined functional group 24b. However, unlike the base material 10b′ for making an analysis sensor of a detection subject of FIG. 3A, the base material 10c′ for making an analysis sensor of a detection subject further comprises the interaction group 25 and the molecular imprint recessed part 21c is limited to those formed by a mold that interacts with the interaction group 25.

When the base materials 10′, 10a′, 10b′, and 10c′ for making an analysis sensor of a detection subject shown in FIG. 1A, FIG. 2A, FIG. 3A, and FIG. 4A are used to make sensors 10, 101, 10a, 10a₁, 10b, and 10c for analyzing a detection subject schematically shown in FIG. 1B-1, FIG. 1B-2, FIG. 2B-1, FIG. 2B-2, FIG. 3B, and FIG. 4B, respectively, at least a specific binding group 42 (small substance group) for a target is bound to the groups 22′ and 22b′ for binding a specific binding molecule to a target, and a signal group 43 is bound to the groups 23′ and 23b′ for binding a signal substance.

### <1-2. Shape>

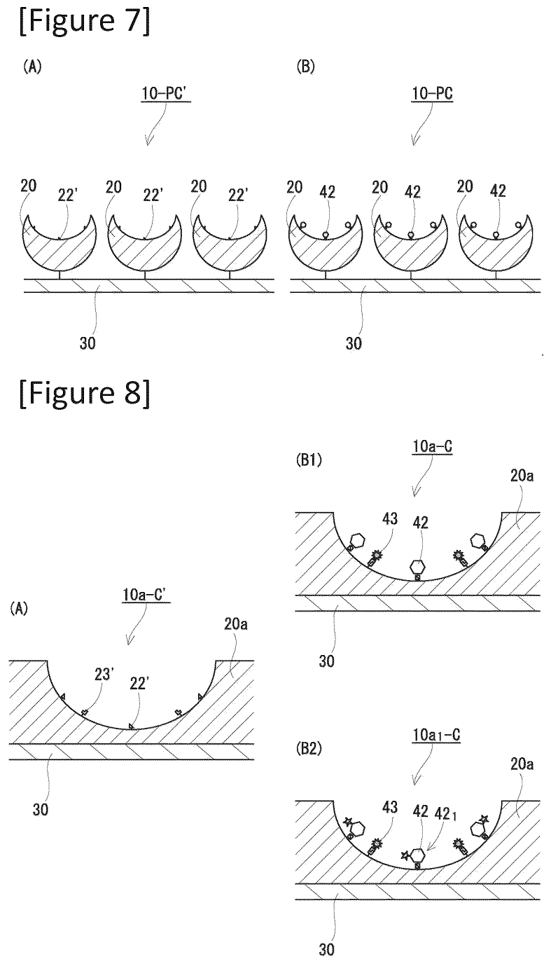

More specific examples of the base material 10′ for making an analysis sensor of a detection subject of the present disclosure shown in FIG. 1A are shown as base materials 10-C′, 10-P′, and 10-PC′ for making an analysis sensor of a detection subject in FIG. 5(A), FIG. 6(A), and FIG. 7(A). The base materials 10-C′, 10-P′, and 10-PC′ for making an analysis sensor of a detection subject of the present disclosure are used for making sensors 10-C, 10-P, and 10-PC for analyzing a detection subject schematically shown in FIG. 5(B), FIG. 6(B), and FIG. 7(B), respectively.

The base material 10-C′ for making an analysis sensor of a detection subject of the present disclosure shown in FIG. 5(A) is an example of the case wherein the base material 10′ for making an analysis sensor of a detection subject of the present disclosure is like a base plate (like a chip). In other words, the base material 10-C′ for making an analysis sensor of a detection subject comprises a base plate 30 and a polymer film provided on a surface of the base plate 30, wherein an imprint polymer 10 configures said polymer film.

The base material 10-P′ for making an analysis sensor of a detection subject of the present disclosure shown in FIG. 6(A) is an example of the case wherein the base material 10′ for making an analysis sensor of a detection subject of the present disclosure is particle-like. In other words, in the base material 10-P′ for making an analysis sensor of a detection subject, the imprint polymer 10 is in a particle-like form.

The base material 10-PC′ for making an analysis sensor of a detection subject of the present disclosure shown in FIG. 7(A) is an example of the case wherein the base material 10′ for making an analysis sensor of a detection subject of the present disclosure is particle-like and immobilized on a base plate (chip). In other words, the base material 10-P′ for making an analysis sensor of a detection subject shown in FIG. 6(A) is immobilized on the base plate 30 in the base material 10-PC′ for making an analysis sensor of a detection subject shown in FIG. 7(A)

A preferable example of the shape of a molecularly imprinted polymer includes a particle-like shape. The point of a molecularly imprinted polymer being particle-like not only enables a sensor for analyzing a detection subject to be in a base plate (chip)-like shape by being immobilized on a base plate like the base material 10-PC′ for making an analysis sensor of a detection subject shown in FIG. 7(A) discussed above, but also enables the form wherein a sensor for analyzing a detection subject is free (e.g., dispersed in a dispersion medium) by being in a non-immobilized state like the base material 10-P′ for making an analysis sensor of a detection subject shown in FIG. 6(A) discussed above, which is excellent in terms of versatility.

While the average particle size of a molecularly imprinted polymer of the case of being particle-like differs in accordance with the size, purpose, and the like of a detection subject or a target of the detection subject, the size includes, in terms of, for example, volume average particle size measured by dynamic light scattering method (DLS), for example, 5 to 500 nm, preferably 8 to 170 nm, more preferably 10 to 100 nm, further preferably 15 to 50 nm, even more preferably 20 to 30 nm. In addition, in terms of Z average particle size measured by DLS, the size includes, for example, 5 to 500 nm, preferably 50 to 350 nm, more preferably 80 to 200 nm, further preferably 100 to 140 nm, even more preferably 120 to 170 nm. Furthermore, in terms of number average particle size measured by a transmission electron microscope (TEM), the size includes, for example, 5 to 150 nm, preferably 10 to 100 nm, more preferably 10 to 35 nm or 45 to 90 nm, further preferably 20 to 30 nm. In addition, the number average particle size measured by a transmission electron microscope (TEM) uses the average value of the long axis and the short axis of the particle as the grain size in the transmission electron microscope, using the average value of said grain size of 100 randomly selected particles.

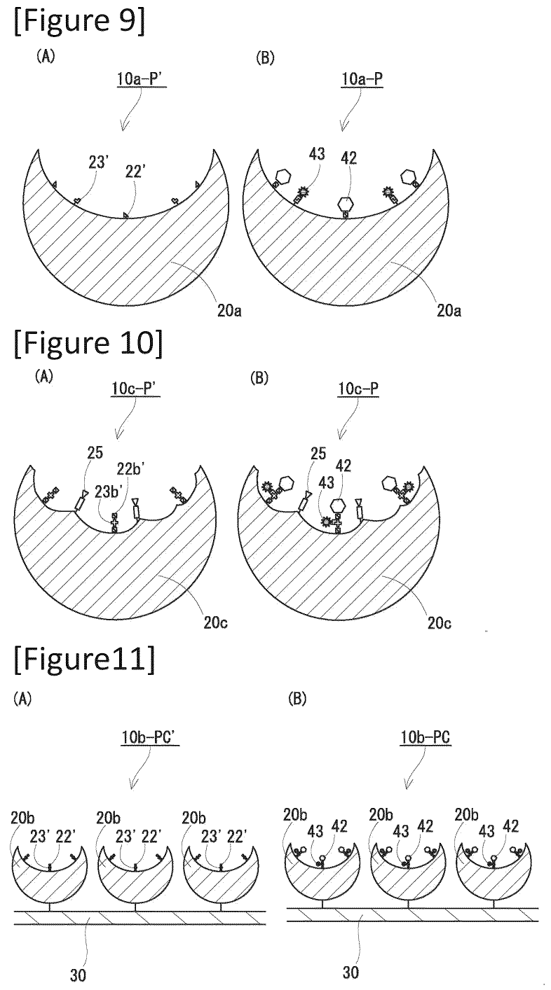

Any of base materials 10a′, 10b′, and 10c′ for making an analysis sensor of a detection subject of the present disclosure shown in FIG. 2A, FIG. 3A, and FIG. 4A may also be configured as being base plate (chip)-like, being particle-like, or being particle-like and immobilized on a base plate. Some of the examples thereof are schematically shown as base materials 10a-C′, 10a-P′, 10c-P′, and 10b-PC′ for making an analysis sensor of a detection subject in FIG. 8(A), FIG. 9(A), FIG. 10(A), and FIG. 11(A), respectively. The base materials for making an analysis sensor of a detection subject of the present disclosure shown in these diagrams are used to make the sensors for analyzing a detection subject schematically shown in FIG. 5(B), FIG. 6(B), FIG. 7(B), FIG. 8(B1) and (B2), FIG. 9(B), FIG. 10(B), FIG. 11(B), respectively.

When base materials 10-C′, 10-P′, 10-PC′, 10a-C′, 10a-P′, 10c-P′, and 10b-PC′ for making an analysis sensor of a detection subject shown in FIG. 5(A), FIG. 6(A), FIG. 7(A), FIG. 8(A), FIG. 9(A), FIG. 10(A), and FIG. 11(A) are used to make the analysis sensors 10-C, 10-P, 10-PC, 10a-C, 10a₁-C, 10a-P, 10c-P, and 10b-PC of a detection subject shown in FIG. 5(B), FIG. 6(B), FIG. 7(B), FIG. 8(B1) and (B2), FIG. 9(B), FIG. 10(B), and FIG. 11(B), at least a specific binding group 42 (small substance group) for a target is bound to groups 22′ and 22b′ for binding a specific binding molecule to a target, and a signal group 43 is bound to groups 23′ and 23b′ for binding a signal substance.

### <1-3. Each Constituent Element>

Each element configuring these base materials for making an analysis sensor of a detection subject is discussed in detail below.

### <1-3-1. Molecular Imprint Recessed Part>

Molecular imprint recessed parts 21 and 21c are portions that provide a sensor field in the sensor for analyzing a detection subject of the present disclosure, which is formed to receive a target of a detection subject. Specifically, the molecular imprint recessed parts 21 and 21c are portions wherein a polymer is molded by a mold (molds 60 and 60c discussed below) used in a molecularly imprinted polymerization method, having the shape that corresponds to a part of the surface shape of said mold. The recessed parts 21 and 21c at least should only be formed in a size that can receive a target of a detection subject. In addition, the recessed parts 21 and 21c have the shape of configuration wherein the molecular imprint recessed part is adapted to a small substance.

The molecular imprint recessed parts 21 and 21c being in a “size that can receive a target of a detection subject” refers to the point that, when the specific binding group 42 for a target is introduced to form a sensor for analysis, the molecular imprint recessed parts 21 and 21c are opened to a sufficient size to the extent that enables at least a part of the detection subject (e.g., at least a specific binding site for the specific binding group 42 in the target 72 of the detection subject 70 discussed below) to enter within the molecular imprint recessed parts 21 and 21c to approach and bind to a specific binding molecule.

Therefore, the recessed parts 21 and 21c may use a substance that is the same size as a detection subject or a target of the detection subject, or a substance of which size is greater than the detection subject, as a mold. Preferably, the recessed parts 21 and 21c use a substance of which size is greater than a detection subject or a target of the detection subject as a mold.

### <1-3-2. A Group for Binding a Specific Binding Molecule to A Target>

The groups 22′ and 22b′ for binding a specific binding molecule to a target are groups that enables introduction of a specific binding molecule to a target by binding a specific binding group for a target (the specific binding group 42 in FIG. 1B-1, FIG. 1B-2, FIG. 2B-1, FIG. 2B-2, FIG. 3B, and FIG. 4B). A user can freely select a target, and can introduce a specific binding molecule for the selected target to the groups 22′ and 22b′ for binding.

Herein, a specific binding molecule is a binding molecule having high affinity/selectivity to a target.

In addition, when the base material for making a sensor for analysis of the present disclosure comprises a base plate 30 (e.g., in the case of base materials 10-C′, 10-PC′, 10a-C′, 10b-PC′, and the like for making an analysis sensor of a detection subject of the present disclosure shown in FIG. 5(A), FIG. 7(A), FIG. 8(A), and FIG. 11(A) in the depicted embodiment), it is also possible to carry out customization so as to enable analysis of a detection subject using a plurality of types of specific binding groups in one base plate 30 by introducing one type of specific binding molecule to one of the recessed part 21 and 21c on one base plate 30 and introducing another type of specific binding molecule to the other of the recessed part 21 and 21c (i.e., one type of specific binding molecule and a molecule with a different specific binding group).

In addition, the specific method of introducing a plurality of types of specific binding molecules to one base plate 30 includes a method of fractionating a region on the base plate 30 into a plurality of regions and introducing different specific binding molecules to each fractionated region, and the like. The method of fractionation includes: a method of providing on a base plate 30 a portion where a polymer film (e.g., the film-like molecularly imprinted polymers 20 and 20a of FIG. 5(A) and FIG. 8(A)) or polymer particle (e.g., the particle-like molecularly imprinted polymers 20 and 20b of FIG. 7(A) and FIG. 11(A)) consisting of a molecularly imprinted polymer is absent; a method of providing a portion where a recess part 21 is absent on a polymer film (e.g., the film-like molecularly imprinted polymers 20 and 20a of FIG. 5(A) and FIG. 8(A)) consisting of a molecularly imprinted polymer; a method of projecting a wall with a polymer or the like on a polymer film (e.g., the film-like molecularly imprinted polymers 20 and 20a of FIG. 5(A) and FIG. 8(A)) consisting of a molecularly imprinted polymer; and the like.

As a specific example of such customization, a specific binding molecule that binds to a surface protein of a specific small extracellular vesicle is introduced to one of the recessed parts 21 and 21c and a specific binding molecule that binds to a specific protein (protein not expressed on the surface of a small extracellular vesicle) is introduced to the other of the recessed parts 21 and 21c to enable analysis of both a small extracellular vesicle and said protein on one base material 30. In addition, as another specific example of customization, a specific binding molecule that binds to a surface protein A of a specific small extracellular vesicle is introduced to one of the recessed parts 21 and 21c and a specific binding molecule that binds to a surface protein B of said specific small extracellular vesicle is introduced to the other recessed part 31 to enable analysis of different types of surface proteins on a small extracellular vesicle on one base material 30. In addition, a specific binding molecule for a substance expressed on an extracellular vesicle A′ derived from a cell strain A is introduced to one recessed part and a specific binding molecule for a substance expressed on an extracellular vesicle B′ derived from a cell strain B is introduced the other to enable analysis of a small extracellular vesicle derived from different cell strains on one base material. These specific examples can be applied not only to different types of surface proteins, but also to different types of surface targets (e.g., protein, sugar chain, and the like).

In the present disclosure, a plurality of groups 22′ and 22b′ for binding a specific binding molecule to a target are provided per one molecule imprint recessed part 21 or 21c.

The groups 22′ and 22b′ for binding a specific binding molecule are selected from groups commonly known as binding functional groups without any particular restriction.

A group that can form a reversible linking group (the first reversible linking group 22 discussed below) is selected among groups generally known as binding functional groups as the group 22′ for binding a specific binding molecule of the groups 22′ and 22b′ for binding a specific binding molecule. A reversible linking group refers to a group of direct binding or bivalent or more wherein cutting (cleavage) and binding are reversible. It does not matter whether the binding form of a reversible portion of the reversible linking group (the first reversible linking group 22 discussed below) is a covalent bond or a non-covalent bond. The specific example of the group 22′ for binding a specific binding molecule includes the groups shown in column 1-1 of Table 1 below, and the specific example of a corresponding reversible linking group (the first reversible linking group 22 discussed below) includes, but not limited to, the groups shown in column 1-3 of Table 1 below.

TABLE 1

| 1-1 | 1-2 | 1-3 | 1-4 |
|---|---|---|---|
| Binding functional group | Binding functional group | Reversible | Form of |
| | | linking group | bond of |
| | | | a reversible |
| | | | portion |
| (Group 22′ for binding | (Binding group 22″) | (First reversible | |
| a specific binding molecule | | linking group 22) | |
| to a target molecule) | | | |
| (Group 23′ for binding | (Binding group 23″) | (Second reversible | |
| a signal substance) | | linking group 23) | |
| (Binding group 26b′) | (Binding group 26b″) | (Third reversible | |
| | | linking group 26b) | |
| thiol group, | thiol group, | disulfide group | Covalent |
| disulfide group, | disulfide group, | | bond |
| pyridyl disulfide group | pyridyl disulfide group | | |
| boronyl group | sugar group | boronic acid cis | Covalent |
| | (cis-diol group) | diol ester | bond |
| carbonyl group/ | amino group | imino bond group | Covalent |
| aldehyde group | | | bond |
| amino group | carbonyl group/ | | |
| | aldehyde group | | |
| carboxyl group | hydroxyl group | carboxylic acid | Covalent |
| hydroxyl group | carboxyl group/ | ester group | bond |
| | carboxylic acid-active | | |
| | ester group | | |
| aminooxy group | carboxy group/ | oxime group | Covalent |
| | aldehyde group | | bond |
| carbonyl group/ | aminooxy group | | |
| aldehyde group | | | |
| carboxy group/ | hydroxyl group | acetal group | Covalent |
| aldehyde group | | | bond |
| hydroxyl group | carboxy group/ | | |
| | aldehyde group | | |
| metal complex | polyhistidine (His) tag | coordinate | non- |
| (Ni-NTA (nickel- | (6 × His, 8 × His, | bond | covalent |
| nitrilotriacetic acid-driven | 10 × His or the like) | | bond |
| group) or the like) | | | |
| basic group | acidic group | hydrogen | non- |
| (amino group, cyclic | (carboxyl group/ | bond | covalent |
| secondary amino group | carboxylic acid-active | | bond |
| (e.g., pyrrolidyl group), | ester group, or the like) | | |
| piperidi group. pyridyl | | | |
| group, imidazole group, | | | |
| guanidine group, | | | |
| or the like) | | | |
| hydroxyl group | | | |
| acidic group | basic group | | non- |
| (carboxyl group, | amino group, cyclic | | covalent |
| or the like) | secondary amino group | | bond |
| | (e.g., pyrroliciyl group, | | |
| | piperidi group), pyridyl | | |
| | group, imidazole group, | | |
| | guanidine group, | | |
| | or the like) | | |
| | hydroxyl group | | |
| aromatic groups | aromatic groups | hydrophobic | non- |
| (phenyl group such as | (phenyl group such as | bond | covalent |
| aminophenyl group, | aminophenyl group, | | bond |
| naphthyl group such as | naphthyl group such as | | |
| aminonaphthyl group, | aminonaphthyl group, | | |
| pyridyl group, or the like) | pyridyl group, or the like) | | |
| hydrazide | carbonyl group/ | hydrazone | covalent |
| | aldehyde group | bond | bond |
| carbonyl group/ | hydrazide | | |
| aldehyde group | | | |
| avidin (NeutrAvidin, | biotin | avidin-biotin | non- |
| streptavidin) | (biocytin, desthiobiotin) | bond | covalent |
| biotin | avidin (NeutrAvidin, | | bond |
| (biocytin, desthiobiotin) | streptavidin) | | |
| glutathione (GSH) | glutathione-S-transferase | glutathione (GSH)- | non- |
| | (GST) | glutathione-S- | covalent |
| | | transferase (GST) | bond |
| glutathione-S-transferase | glutathione (GSH) | bond | |
| (GST) | | | |
| Phos-tag | phosphate ion | coordinate bond | non- |
| phosphate ion | Phos-tag | | covalent |
| | | | bond |

Among these binding functional groups, the group 22′ for binding a specific binding molecule is preferably a reversible linking group with a covalent bond or a coordinate bond. A reversible linking group with a covalent bond or a coordinate bond is relatively stable compared to a hydrogen bond and a hydrophobic bond, which is stably maintained also at the time of polymerization reaction, which makes formation of the molecular imprint recessed parts 21 and 21c easy, but is also easily cut by a reducing agent, heating with a relatively low temperature, hydrolysis under a relatively mild condition, light irradiation, or the like, and thus removal of a mold after polymerization reaction is also easy.

Furthermore, among these binding functional groups, a suitable example of the group 22′ for binding a specific binding molecule includes a metal complex, more preferably a nickel complex, further preferably a nickel-nitrilotriacetic acid-driven group, and the like. The case wherein such group 22′ for binding a specific binding molecule is suitable includes the case wherein a molecularly imprinted polymer of a base material for making an analysis sensor of a detection subject configures a polymer film (e.g., the case of the base materials 10-C′, 10a-C′ and the like for making an analysis sensor of a detection subject shown in FIG. 5(A) and FIG. 8(A)).

In addition, among these binding functional groups, other suitable examples of the group 22′ for binding a specific binding molecule includes a thiol group or a pyridyl disulfide group, more preferably a thiol group. The case wherein such group 22′ for binding a specific binding molecule is suitable includes the case wherein a molecularly imprinted polymer of a base material for making an analysis sensor of a detection subject is polymer-like (e.g., the case of base materials 10-P′, 10-PC′, 10a-P′ and the like for making an analysis sensor of a detection subject shown in FIG. 6(A), FIG. 7(A) and FIG. 9(A)).

Between the groups 22′ and 22b′ for binding a specific binding antibody, a group that can form a reversible or non-reversible linking group (the linking group 22b discussed below) is selected among groups commonly known as binding functional groups as the group 22b′ for binding a specific binding molecule. From the viewpoint of stably binding a specific binding molecule (the specific binding molecule R42 discussed below), the group 22b′ for binding a specific binding molecule is preferably a group that can form a reversible or non-reversible linking group (the linking group 22b disclosed below) with a covalent bond. The specific example of such group 22b′ for binding a specific binding molecule includes, but not limited to, the groups shown in column 2-1 of Table 2 below.

TABLE 2

| 2-1 | 2-2 |
|---|---|
| Binding functional group | Corresponding binding functional group |
| (Group 22b′ for binding a specific binding | (Binding group 22b″) |
| molecule to a target molecule) | |
| (Group binding group 23b for binding | (Binding group 23b″) |
| a signal substance) | |
| (Binding groups BG22″, BG23″, BG1′, and BG6″) | (Binding groups BG22′, BG23′, BG1″, and BG6′) |
| amino group (monovalent amino group, | carboxylic acid-active ester group |
| bivalent amino group, or the like) | (active ester group using N- |
| | hydroxysuccinimide, nitrophenol, or |
| | pentafluorophenol; carbamic acid active |
| | ster group such as NHS carbamate); |
| | carboxyl group; aldehyde group; |
| | isocyanate group; isothiocyanate group; |
| | epoxy group; maleimide group; or the like |
| amino group | carboxyl group |
| carboxyl group | amino group |
| sugar group (cis-diol group) | boronyl group |
| boronyl group | sugar group (cis-diol group) |
| thiol group | unsaturated carbon group lacking an enectron |
| | such as maleimide group or acrylate ester; |
| | iodoacetamide group; thiol group; |
| | pyridyl disulfide group; alkene (vinyl sulfone)/ |
| | alkyne group for radical addition click |
| | chemistry; a thioester group for a native |
| | chemical ligation method; or the like |
| unsaturated carbon group lacking an enectron | thiol group |
| such as maleimide group or acrylate ester; | |
| iodoacetamide group; thiol group; pyridyl disulfide | |
| group; alkene (vinyl sulfone) chemistry; a thioester | |
| group for a native chemical ligation method; | |
| or the like | |
| carbonyl group/aldehyde group | amino group |
| amino group | carbonyl group/aldehyde group |
| aminooxy group | carbonyl group/aldehyde group |
| carbonyl group/aldehyde group | aminooxy group |
| carbonyl group/aldehyde group | hydroxyl group |
| hydroxyl group | carbonyl group/aldehyde group |
| hydroxyl group, phenolic hydroxyl group | carboxylic acid-active ester group |
| hydrazide | carbonyl group/aldehyde group |
| carbonyl group/aldehyde group | hydrazide |
| avidin (NeutrAvidin, streptavidin) | biotin (blocytin, desthiobiotin) |
| biotin (biocytin, desthiobiotin) | avidin (NeutrAvidin, streptavidin) |
| glutathione (GSH) | glutathione-S-transferase |
| glutathione-S-transferase | glutathione (GSH) |

In the present disclosure, chemical modification may be carried out to a binding group made by being chemically modified.

Among these binding functional groups, suitable examples of the group 22b′ for binding a specific binding molecule includes a thiol group or a pyridyl disulfide group, more preferably a pyridyl disulfide group.

### <1-3-3. A Group for Binding a Signal Substance>

The groups 23′ and 23b′ for binding a signal substance are groups that can introduce a signal substance by binding a signal group (the signal group 43 of FIG. 2B-1, FIG. 2B-2, FIG. 3B and FIG. 4B). A user can freely select a signal substance, and introduction to the groups 23′ and 23b′ is possible.

In the present disclosure, it is possible to provide a plurality of groups 23′ and 23b′ for binding a signal substance per one molecular imprint recessed part 21 or 21c.

Each of the groups 23′ and 23b′ for binding a signal substance is selected from those commonly known as binding functional groups with no particular restriction as long as the groups are different from the groups 22′ and 22b′ for binding a specific binding molecule.

Among the groups 23′ and 23b′ for binding a signal substance, a group that can form a reversible linking group (the second reversible linking group 23 discussed below) is selected among groups commonly known as binding functional groups as the group 23′ for binding a signal substance. It does not matter whether the binding form of the reversible portion of the reversible linking group (the second reversible linking group 23 discussed below) is a covalent bond or a non-covalent bond. A specific example of the group 23′ for binding a signal substance includes the groups shown in column 1-1 of Table 1 above, and a specific example of a corresponding reversible linking group (the second reversible linking group 23 discussed below) includes the groups shown in column 1-3 of Table 1 above.

Among these binding functional groups, the group 23′ for binding a signal substance is preferably a group that forms a reversible linking group with a covalent bond or a coordinate bond. A reversible linking group with a covalent bond or a coordinate bond is relatively stable compared to a hydrogen bond and a hydrophobic bond, which is stably maintained also at the time of polymerization reaction, which makes formation of the molecular imprint recessed parts 21 and 21c easy, but is also easily cut by a reducing agent, heating with a relatively low temperature, hydrolysis under a relatively mild condition, light irradiation, or the like, and thus removal of a mold after polymerization reaction is also easy.

Furthermore, among these binding functional groups, an appropriate example of the group 23′ for binding a signal substance includes a group that forms a reversible linking group with a covalent bond, more preferably a thiol group or a pyridyl disulfide group, further preferably a thiol group.

Among groups 23′ and 23b′ for binding a signal substance, a group that can form a reversible or non-reversible linking group (the linking group 23b discussed below) is selected among groups commonly known as binding functional groups as the group 23b′ for binding a signal substance. From the viewpoint of stably binding a signal substance (the signal substance R43 discussed below), the group 23b′ for binding a signal substance is preferably a group that can form a reversible or non-reversible linking group (the linking group 23b discussed below) with a covalent bond. A specific example of such a group 23b′ for binding a signal substance includes the groups shown in column 2-1 of Table 2 above.

Among these binding functional groups, a suitable example of the group 23b′ for binding a signal substance includes an amino group.

### <1-3-4. Functional Group>

The above-mentioned group 22b′ for binding a specific binding molecule and the group 23b′ for binding a signal substance configure a functional group 24b. Since the functional group 24b comprises both the group 22b′ for binding a specific binding molecule and the group 23b′ for binding a signal substance in one group, it is easy to closely provide these groups to a base material for making an analysis sensor of a detection subject. The act of closely providing the group 22b′ for binding a specific binding molecule and the group 23b′ for binding a signal substance in the base material for making an analysis sensor of a detection subject is, in other words, the act of closely providing a specific binding group 42 and a signal group 43 in an analysis sensor of a detection subject, whereby it is possible to more sensitively obtain signal change when the analysis sensor of a detection subject recognizes a detection subject.

In the depicted embodiment, while the group 22b′ for binding a specific binding molecule is positioned on the tip side of the functional group 24b and the binding group 23b′ of a signal substance is positioned on the base end side of the functional group 24b, the positional relationship between the group 22b′ for binding a specific binding molecule and the binding group 23b′ of a signal substance in the functional group 24b is not limited thereto as long as the positional relationship enables specific binding of the specific binding group 42 bound to the group 22b′ for binding a specific binding molecule to a target of the detection subject and causes the signal group 43 bound to the group 23b′ for binding a signal substance to receive an environmental change upon specific binding. For example, it is possible to reverse with an embodiment wherein the positional relationship between the group 22b′ for binding a specific binding molecule and the binding group 23b′ of a signal substance is depicted. However, from the viewpoint of obtaining a higher recognition capacity by achieving an easier specific binding between a target of a detection subject and the specific binding group 42 and/or from the viewpoint of more highly sensitively obtaining a signal change by more certainly changing the environment of the signal group 43 upon the specific binding between a target of a detection subject and a specific binding group 42, it is preferable that the group 22b′ for binding a specific binding molecule is positioned on the tip side of the functional group 24b and the binding group 23b′ of a signal substance is positioned on the base end side of the functional group 24b as depicted.

A more specific structure of the functional group 24b includes the group represented by Formula (1) below.

[Chemical 7]

—R²-L¹-R¹ (1)

In Formula (1), R² represents a group for binding a specific binding molecule, L² represents a direct binding or linking group, and R² represents a linking group comprising a group for binding a signal substance.

The group R² for binding a specific binding molecule is as discussed in “1-3-2. A group for binding a specific binding molecule to a target” above.

The direct binding or linking group L² is interposed between the group R² for binding a specific binding molecule and the linking group R² comprising a group for binding a signal substance. It is more preferable that L² is a linking group from the viewpoint of more effectively suppressing interference of the signal group 43 that binds to a group for binding a signal substance comprised in the linking group R² and specific recognition of a detection subject relative to the specific binding group 42 that binds to the group R² for binding a specific binding molecule when a sensor for analyzing a detection subject recognizes a detection subject.

When L¹ is a linking group, said linking group includes an alkylene group (e.g., C1 to 8 alkylene group, or polyoxy C1 to 3 alkylene C1 to 8 alkylene group) that may comprise a polyoxyalkylene group, carbonyl group, ester bond (—COO— or —OCO—), amide bond (—CONH— or —NHCO—), and a group consisting of any combination thereof. From the viewpoint of molecular design and the like, said linking group includes, preferably a combination of an alkylene group that may comprise a polyoxyalkylene group and an ester bond and/or an amide bond, more preferably a combination of an alkylene group that may comprise a polyoxyalkylene group and an ester bond or an amide bond, further preferably a combination of one or two or more alkylene group (the total of the carbon number of all alkylene groups comprised in said linking group is, for example, 1 to 8, preferably 2 to 6, more preferably 3 to 5, especially preferably 4) and an ester bond or an amide bond.

The group for binding a signal substance in the linking group R² comprising the group for binding a signal substance is as discussed in “1-3-3. A group for binding a signal substance” above. The linking group R² comprising a group for binding a signal substance is a non-branched or branched bivalent group.

When the linking group R² is non-branched, a signal substance binding group itself makes a linking group R² (i.e., the linking group R² consists of a signal substance binding group). Such a non-branched linking group R², i.e., a signal substance binding group, includes the bivalent amino group represented by Formula (2) below.

[Chemical 8]

—NR³— (2)

In Formula (2), R³ represents hydrogen or an alkyl group with the carbon number of 1 to 8 that may comprise a polyoxyalkylene group (e.g., C1 to 8 alkylene group, or polyoxy C1 to 3 alkylene C1 to 8 alkylene group), preferably hydrogen.

When the linking group R² is branched, the signal substance binding group is a monovalent group that exists in a branch. Such a linking group R² includes the bivalent group represented by Formula (3) below.

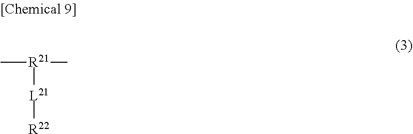

In Formula (3), R²² represents nitrogen atom or carbohydrate, L²¹ represents a direct binding or linking group, and R²² represents a group for binding a signal substance.

When nitrogen atom or carbohydrate R²² is carbohydrate, R²¹ is carbohydrate represented by —C—R²¹¹. Although not particularly limited, R²¹¹ includes, for example, hydrogen atom or an alkyl group with the carbon number of 1 to 8 that may comprise a polyoxyalkylene group (e.g., C1 to 8 alkyl group, or polyoxy C1 to 3 alkylene C1 to 8 alkyl group), preferably hydrogen atom.

From the viewpoint of more effectively suppressing interference of the signal group 43 that binds to the group R²² for binding a signal substance and specific recognition of a detection subject with respect to the specific binding group 42 that binds to the group R² for binding a specific binding molecule when a sensor for analyzing a detection subject recognizes a detection subject, the direct binding or linking group L²¹ is more preferably a linking group.

When the direct binding or linking group L²¹ is a linking group, said linking group includes an alkylene group (e.g., C1 to 8 alkylene group, or polyoxy C1 to 3 alkylene C1 to 8 alkylene group) that may comprise a polyoxyalkylene group, carbonyl group, ester bond (—COO— or —OCO—), amide bond (—CONH— or —NHCO—), and a group consisting of any combination thereof. From the viewpoint of molecular design and the like, said linking group includes preferably an alkylene group that may comprise a polyoxyalkylene group, more preferably an alkylene group with the carbon number of 1 to 6 (preferably 1 to 4, more preferably 1 to 3, especially preferably 2).

The group R²² for binding a signal substance is as discussed in “1-3-3. A group for binding a signal substance” above.

A specific example that is not limited to the group represented by the Formula (1) above includes the following group.

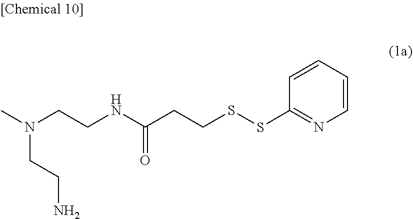 [SMILES: CN(CCN)CCNC(=O)CCSSc1ccccn1]

### <1-3-5. Interaction Group>

The interaction group 25 in the base material 10c for making an analysis sensor of a detection subject shown in FIG. 4A is a group that interacts with an interaction site (the interaction site 65c discussed below) in a mold of the molecular imprint recessed part 21c. The mold (the mold having the interaction site 65c discussed below) corresponding to the interaction group 25 includes a biomolecule such as protein or glycoprotein.

A specific example of the interaction group 25 is designed in accordance with the surface state of a corresponding mold, i.e., an interaction group of the mold. For example, the specific example includes a group that can form a hydrogen bond, electrostatic interaction, or weak covalent bond, or the like due to the interaction with a specific amino acid residue or sugar residue, or the like configuring a protein, or glycoprotein, or the like that forms the mold, and a group that can form a hydrophobic bond with a specific hydrophobic region of a protein that forms a mold, and the like.

A specific example of the interaction group 25 includes <1> a substituted or unsubstituted amino group, <2> a substituted or unsubstituted aromatic group, <3> an amidino group (—C(═NH)NH₂), <4> a guanidino group (—NHC(═NH)NH₂), <5> a carboxyl group, <6> a sulfo group, and/or <7> a bornyl group (—B(OH)₂). The aromatic group in <2> the substituted or unsubstituted aromatic group comprises <21> a substituted or unsubstituted nitrogen-containing aromatic group and <22> a substituted or unsubstituted nitrogen-free aromatic group, and includes <25> a substituted or unsubstituted aryl group (<251> a nitrogen-containing aryl group and <252> a nitrogen-free aryl group) and <26> a substituted or unsubstituted arylene group (<261> a nitrogen-containing arylene group and <262> a nitrogen-free arylene group). The interaction group 25 may be a group consisting of any of these groups, and may be a group comprising a combination of two or more types selected from these groups. The combination of the interaction group 25, a corresponding interaction site (the interaction site 65c discussed below), and the type of non-covalent bond formed by the interaction thereof is shown in the Table below, but the combination is not limited thereto.

TABLE 3

| 3-1 | 3-2 | 3-3 |
|---|---|---|
| Interaction group 25 | Interaction site 65c | Type of non-covalent bond |
| | in a mold | |
| basic acid (substitued or unsubstituted | acidic amino acid residue | Non-covalent bond |
| amino group, substituted or unsubstituted | carboxyl group or the like) | (hydrogen bond, |
| nitrogen-containing aromatic group, | | electrostatic interaction) |
| amidino group, guanidion group, or the like) | | |
| acidic group | acidic amino acid residue | Non-covalent bond |
| (carboxyl group, sulfo group) | (amino group or the like) | (hydrogen bond, |
| | | electrostatic interaction) |
| substituted or unsubstituted | aromatic amino acid residue | Non-covalent bond |
| aromatic group (substituted or | (phenyl group, indol group, | (hydrophobic interaction) |
| unsubstituted aryl group, | imidazoyl group, or the like) | |
| arylene group, or the like) | | |
| substituted or unsubstituted | hydrophobic region | Non-covalent bond |
| aromatic group (substituted or | | (hydrophobic interaction) |
| unsubstituted aryl group, | | |
| arylene group, or the like) | | |
| bornyl group | sugar group (cis diol group) | Weak covalent bond |
| | | (boronic acid cis diol |
| | | ester group) |

In <1> the substituted or unsubstituted amino group, a substituent in the substituted amino group includes: a straight chain or branched alkyl group, substituted or unsubstituted aralkyl group with the carbon number of 1 to 8; an alkenyl group with the carbon number of 4 to 6 that configures a cyclic secondary amino group; a substituted or unsubstituted aryl group; and the like. A specific example of the substituted or unsubstituted aryl group includes groups included in <251> the substituted or unsubstituted nitrogen-containing aryl group and <252> the substituted or unsubstituted nitrogen-free aryl group discussed below. A specific example of <1> the substituted or unsubstituted amino group includes: amino group (unsubstituted); secondary amino groups such as N-methylamino group, N-ethylamino group, N-n-propylamino group, N-isopropylamino group, N-n-butylamino group, N-isobutylamino group, N-tert-butylamino group, N-benzylamino group, N-phenylamino group, N-mesylamino group, and N-tosylamino group; secondary amino groups such as N,N-dimethylamino group, N,N-diethylamino group, N,N-dibenzylamino group, N-ethyl-N-methylamino group, N,N-di-n-propylamino group, N,N-diisopropylamino group, N,N-diphenylamino group, N-methyl-N-phenylamino group, N-mesyl-N-benzylamino group, and N-mesyl-N-methylamino group; cyclic secondary amino groups such as piperidyl group, and pyrrolidyl group; and the like. Among these substituted or unsubstituted amino groups, preferably a cyclic secondary amino group is included, and more preferably a pyrrolidyl group is included.

Among <2> the substituted or unsubstituted aromatic groups, the nitrogen-containing aryl group in <251> the substituted or unsubstituted nitrogen-containing aryl group includes a nitrogen-containing aryl group with the carbon number of 2 to 12. The substituent in the substituted nitrogen-containing aryl group includes a straight chain or branched alkyl group with the carbon number of 1 to 8, and the like. A specific example of <251> the substituted or unsubstituted nitrogen-containing aryl group includes a pyridyl group, pyrimidyl group, pyridazyl group, pyrazyl group, imidazolyl group, triazolyl group, methylpyridyl group (2-methylpyridyl group, 3-methylpyridyl group, 4-methylpyridyl group and the like), dimethylpyridyl group (2,6-dimethylpyridyl group and the like), methylethylpyridyl group (2-methyl-6-ethylpyridyl group and the like), methylimidazolyl group (1-methylimidazolyl group and the like), dimethylimidazolyl group (1,2-dimethylimidazolyl group and the like), ethylimidazolyl group (1-ethylimidazolyl group and the like), propylimidazolyl group (1-n-propylimidazolyl group and the like), brilimidazolyl group (1-n-butylimidazolyl group and the like), pentylimidazolyl group (1-n-pentylimidazolyl group and the like), hexylimidazolyl group (1-n-hexylimidazolyl and the like), and the like.

Among <2> the substituted or unsubstituted aromatic groups, the nitrogen-free aryl group in <252> the substituted or unsubstituted nitrogen-free aryl group includes a phenyl group and naphthyl group (1-naphthyl group, 2-naphthyl group, and the like) with the carbon number of 6 to 16, and the like. The substituent in the substituted aryl group includes a straight chain or branched alkyl group, nitro group, halogen group (fluoro group, chloro group, bromo group, and the like) with the carbon number of 1 to 8, and the like. A specific example of <252> the substituted or unsubstituted aryl group includes a phenyl group, naphthyl group, tolyl group (o-tolyl group, m-tolyl group, p-tolyl group and the like), ethylphenyl group (4-ethylphenyl group, 3-ethylfail group, 2-ethylphenyl group, and the like), propylphenyl group (4-n-propylphenyl group, and the like), isopropylphenyl group (4-isopropylphenyl group, 2-isopropylphenyl group, and the like), butylphenyl group (4-n-butylphenyl group, 4-isobutylphenyl group, 4-t-butylphenyl group, 3-t-butylphenyl group, 2-t-butylphenyl group, and the like), pentylphenyl group (4-n-pentylphenyl group, 4-isopentylphenyl group, 2-neopentylphenyl group, 4-t-pentylphenyl group, and the like), hexylphenyl group (4-n-hexylphenyl group and the like), 4-(2-ethylbutyl)phenyl group, 4-n-heptylphenyl group, 4-n-octylphenyl group, 4-(2-ethylhexyl)phenyl group, 4-t-octylphenyl group, 4-ethyl-1-naphthyl group, 6-n-butyl-2-naphthyl group, dimethylphenyl group (2,4-dimethylphenyl group, 2,5-dimethylphenyl group, 3,4-dimethylphenyl group, 3,5-dimethylphenyl group, 2,6-dimethylphenyl group, and the like), trimethylphenyl group (2,3,5-trimethylphenyl group, 2,3,6-trimethylphenyl group, 3,4,5-trimethylphenyl group and the like), diethylphenyl group (2,4-diethylphenyl group, 2,6-diethylphenyl group, and the like), 2,5-diisopropylphenyl group, 2,6-diisobutylphenyl group, di-t-butylphenyl group (2,4-di-t-butylphenyl group, 2,5-di-t-butylphenyl group, and the like), 4,6-di-t-butyl-2-methylphenyl group, 5-t-butyl-2-methylphenyl group, 4-t-butyl-2,6-dimethylphenyl group, fluorophenyl group (4-fluorophenyl group, 3-fluorophenyl group, 2-fluorophenyl group, and the like), chlorophenyl group (4-chlorophenyl group, 3-chlorophenyl group, 2-chlorophenyl group, and the like), bromophenyl group (4-bromophenyl group, 2-bromophenyl group, and the like), chloronaphthyl group (4-chloro-1-naphthyl group, 4-chloro-2-naphthyl group, and the like), 6-bromo-2-naphthyl group, dichlorophenyl group (2,3-dichlorophenyl group, 2,4-dichlorophenyl group, 2,5-dichlorophenyl group, 3,4-dichlorophenyl group, 3,5-dichlorophenyl group, and the like), 2,5-dibromophenyl group, 2,4,6-trichlorophenyl group, dichloronaphthyl group (2,4-dichloro-1-naphthyl group, 1,6-dichloro-2-naphthyl group, and the like), chloromethylphenyl group (2-chloro-4-methylphenyl group, 2-chloro-5-methylphenyl group, 2-chloro-6-methylphenyl group, 3-chloro-4-methylphenyl group, 2-methyl-3-chlorophenyl group, 2-methyl-4-chlorophenyl group, 3-methyl-4-chlorophenyl group, and the like), trifluoromethylphenyl group (4-trifluoromethylphenyl group, and the like), nitrophenyl group (4-nitrophenyl group and the like), and the like.

In addition, a group comprising a combination of two or more types selected from the groups of the above-mentioned <1> to <7> includes a group comprising a combination of a monovalent group among the groups of <1> to <7>, which is <1> the substituted or unsubstituted amino group, <251> the nitrogen-containing aryl group, <252> the nitrogen-free aryl group, <3> the amidino group, <4> the guanidino group, <5> the carboxyl group, <6> the sulfo group, or <7> the bornyl group and <26> the substituted or unsubstituted arylene group (<261> the nitrogen-containing arylene group, <262> nitrogen-free arylene group).

Among <26> the substituted or unsubstituted arylene groups, <261> the nitrogen-containing arylene group is a group wherein one hydrogen bound to a carbon configuring an aromatic ring is removed from a group included as <251> the substituted or unsubstituted nitrogen-containing aryl group described above. Among <26> the substituted or unsubstituted arylene groups, <262> the nitrogen-free arylene groups is a group wherein a hydrogen bound to a carbon configuring aromatic ring is removed from a group included as <252> the substituted or unsubstituted nitrogen-free aryl group described above.

A specific group comprising a combination of two or more types selected from the groups <1> to <7> above includes, an amidinoaryl group which is a combination of <3> an amidino group and <26> a substituted or unsubstituted arylene group, a carboxyaryl group which is a combination of <5> a carboxyl group and <26> a substituted or unsubstituted arylene group, a sulfoaryl group which is a combination of <6> a sulfo group and <26> a substituted or unsubstituted arylene group, a boronylaryl group which is a combination of <7> a boronyl group and <26> a substituted or unsubstituted arylene group, and the like. Preferable groups thereamong are shown below.

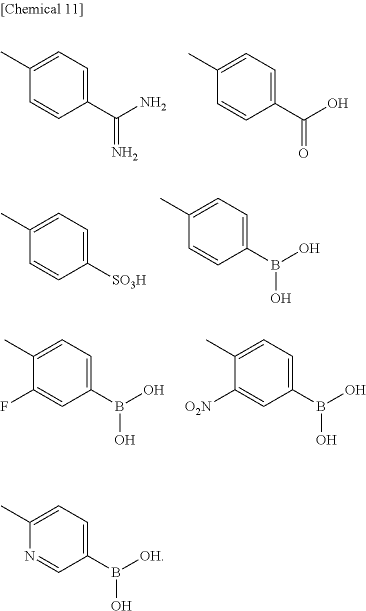

### <1-3-6. A Polymer Configuring a Molecularly Imprinted Polymer>

The constituent component of a polymer configuring molecularly imprinted polymers 20, 20a, 20b and 20c is not particularly limited. For example, said polymer comprises a constituent unit selected from the group consisting of a unit derived from a biocompatible monomer, a unit derived from N-substituted or unsubstituted (meth)acrylamide, and a unit derived from a functional monomer having the above-mentioned interaction group 25. In addition, the present specification describes a polymer synthesized by imprinting polymerization using a mold as “molecularly imprinted polymer” for convenience, wherein the present disclosure also allows a matter that is not a molecule (e.g., a microparticle having a film structure) as a mold, and thus the “molecularly imprinted polymer” also encompasses a polymer synthesized by imprinting polymerization using a matter that is not a molecule as a mold.

(A Unit Derived from a Biocompatible Monomer)

A polymer configuring molecularly imprinted polymers 20, 20a, 20b and 20c preferably at least comprises a component derived from a biocompatible monomer. Biocompatibility refers to a property that does not induce adherence of a biological substance. Non-specific adsorption to the molecularly imprinted polymers 20, 20a, 20b and 20c surface can be favorably suppressed by comprising a component derived from a biocompatible monomer (e.g., exemplified as the biocompatible monomer M28 discussed below). A biocompatible monomer preferably includes a hydrophilic monomer, more preferably includes a zwitterionic monomer.

The zwitterionic monomer comprises both an anion group derived from an acidic functional group (e.g., a phosphate group, a sulfate group, and a carboxyl group, and the like) and a cationic group derived from a basic functional group (e.g., a primary amino group, a secondary amino group, a tertiary amino group, and a quaternary ammonium group, and the like) in one molecule. For example, phosphobetaine, sulfobetaine, and carboxybetaine, and the like are included.

More specifically, phosphobetaine includes a molecule having a phosphorylcholine group on a side chain, preferably includes 2-methacryloyloxyethyl phosphorylcholine (MPC) and the like.

Sulfobetaine includes N,N-dimethyl-N-(3-sulfopropyl)-3′-methacryloylaminopropanaminium inner salt (SPB), N,N-dimethyl-N-(4-sulfobutyl)-3′-methacryloylaminopropanaminium inner salt (SBB), and the like.

Carboxybetaine includes N,N-dimethyl-N-(1-carboxymethyl)-2′-methacryloyloxyethaneaminium inner salt (CMB), N,N-dimethyl-N-(2-carboxyethyl)-2′-methacryloyloxyethaneaminium inner salt (CEB), and the like.

Among these zwitterionic monomers, preferably phosphobetaine is included, and more preferably 2-methacryloyloxyethyl phosphorylcholine (MPC) is included.

The ratio of the unit derive from a biocompatible monomer in the entire constituent unit of the molecularly imprinted polymers 20, 20a, 20b and 20c includes, for example, 1 mol % or greater and 100 mol % or less. The content of the unit derived from a biocompatible monomer is preferably at the above-mentioned lower limitation or greater in terms of suppressing non-specific adsorption in the molecularly imprinted polymers 20, 20a, 20b and 20c surface. In addition, a preferable range of the above-mentioned ratio of the unit derived from a biocompatible monomer may differ depending on the shape of the molecularly imprinted polymers 20, 20a, 20b and 20c. For example, when the molecularly imprinted polymers 20, 20a, 20b and 20c are film-like, the preferable range of the above-mentioned ratio of the unit derived from a biocompatible monomer includes 30 mol % or greater, more preferably 50 mol % or greater, further preferably 70 mol % or greater, even more preferably 80 mol % or greater, further even more preferably 90 mol % or greater, especially preferably 95 mol % or greater. In addition, when the molecularly imprinted polymers 20, 20a, 20b and 20c are particle-like, the preferable range of the above-mentioned ratio of the unit derived from a biocompatible monomer includes, 1 to 50 mol %, preferably 2 to 30 mol %, more preferably 3 to 10 mol %, further preferably 4 to 6 mol %, especially preferably 4.5 to 5.5 mol %.

(A Unit Derived from N-Substituted or Unsubstituted (Meth)Acrylamide)

A polymer configuring the molecularly imprinted polymers 20, 20a, 20b and 20c can comprise a unit derived from N-substituted or unsubstituted (meth)acrylamide. In particular, when the molecularly imprinted polymers 20, 20a, 20b and 20c are particle-like, it is preferable to comprise a unit derived from N-substituted or unsubstituted (meth)acrylamide in the constituent polymer.

N-substituted or unsubstituted (meth)acrylamide (e.g., exemplified as the polymerizable monomer M29 discussed below) is shown in the following Formula (4).

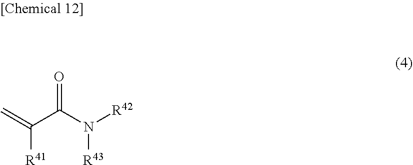

In Formula (4), R⁴¹ represents hydrogen or a methyl group, preferably hydrogen, and R⁴² and R⁴³ each independently represents hydrogen; a straight chain-like or branched chain-like alkyl group with a carbon number of 1 to 6; a straight chain-like or branched chain-like hydroxyalkyl group with a carbon number of 1 to 6; or a straight chain-like or branched chain-like aminoalkyl group with a carbon number of 1 to 6, wherein they may be the same or different from one another; and R⁴² and R⁴³ may form a saturated 5 to 7 membered ring comprising a nitrogen atom carrying them as well as an oxygen atom.

A straight chain-like or branched chain-like alkyl group with a carbon number of 1 to 6 includes a methyl group, ethyl group, n-propyl group, isopropyl group, n-butyl group, sec-butyl group, tert-butyl group, n-pentyl group, n-hexyl group, and the like. A straight chain-like or branched chain-like hydroxyalkyl group with a carbon number of 1 to 6 includes hydroxymethyl group, 1-hydroxyethyl group, 2-hydroxyethyl group, 1-hydroxy-n-propyl group, 2-hydroxy-n-propyl group, 3-hydroxy-n-propyl group, 1-hydroxyisopropyl group, 2-hydroxyisopropyl group, 1-hydroxy-n-butyl group, 1-hydroxy-n-pentyl group, 1-hydroxy-n-hexyl group, and the like. A straight chain-like or branched chain-like aminoalkyl group with a carbon number of 1 to 6 includes aminomethyl group, 1-aminoethyl group, 2-aminoethyl group, 1-amino-n-propyl group, 2-amino-n-propyl group, 3-amino-n-propyl group, 1-aminoisopropyl group, 2-aminoisopropyl group, 1-amino-n-butyl group, 1-amino-n-pentyl group, 1-amino-n-hexyl group, and the like. A saturated 5 to 7 membered ring comprising a nitrogen atom and an oxygen atom includes a morpholine ring, and the like.

Among these N-substituted or unsubstituted (meth)acrylamide, N-substituted (meth)acrylamide is preferably included, more preferably N-mono-substituted (meth)acrylamide is included, further preferably N-alkyl(meth)acrylamide (the alkyl group is the above-mentioned straight chain-like or branched chain-like alkyl group with a carbon number of 1 to 6) is included, even more preferably N-isopropyl(meth)acrylamide is included, and especially preferably N-isopropyl acrylamide is included.

When a unit derived from N-substituted or unsubstituted (meth)acrylamide is comprised in a polymer configuring the molecularly imprinted polymers 20, 20a, 20b and 20c, the ratio of the unit derived from N-substituted or unsubstituted (meth)acrylamide in the entire constituent unit of the molecularly imprinted polymers 20, 20a, 20b and 20c includes, for example, 1 to 99 mol %, preferably 10 to 98 mol %, more preferably 30 to 96 mol %, further preferably 50 to 94 mol %, even more preferably 70 to 92 mol %, further even more preferably 80 to 90 mol %, especially preferably 85 to 90 mol %.

### (A Unit Derived Form a Functional Monomer)

A polymer configuring the molecularly imprinted polymer 20c comprises a unit derived from a functional monomer. The functional monomer is not particularly limited as long as the monomer has the interaction group 25 discussed in “1-3-5. Interaction group” above (e.g., exemplified as the monomer M25 comprising an interaction group for a mold discussed below).

The monomer shown in Formula (5) below is included as an unlimited example of a functional monomer.

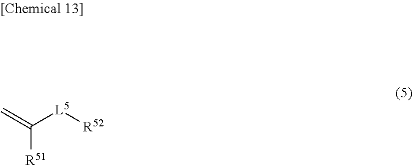

In Formula (5), R5′ represents hydrogen or methyl group, preferably hydrogen, L⁵ represents direct binding or linking group, R⁵² represents the interaction group 25 discussed in “1-3-5. Interaction group” above.

When the direct binding or linking group L⁵ is a linking group, said linking group includes, for example, with the carbon number of 1 to 6, preferably 2 to 6, an alkylene group, amino group (—NH—), ether group (—O—), carbonyl group (—C(═O)—), ester group (—C(═O)—O— or —O—C(═O)—), amide group (—C(═O)—NH— or —NH—C(═O)—), sulfoxide group (—S(═O)—), sulphonyl group (—S(═O)₂—), and a group wherein two or more of the above are bound. Among these linking groups, an ester group (—C(═O)—O) is preferably included.

The specific unlimited example of the monomer represented by Formula (5) above includes the following monomer.

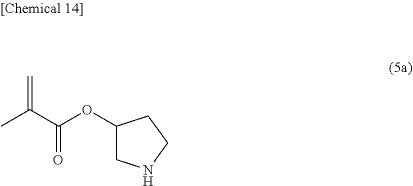

In the molecularly imprinted polymer 20c, a unit derived from a functional monomer at least exists on the recessed part 21c surface. In the molecularly imprinted polymer 20c, a unit derived from a functional monomer may exist not only on the recessed part 21c surface, but also inside. The ratio of the unit derived from a functional monomer in the entire constituent unit of a polymer configuring the molecularly imprinted polymer 20c is not particularly limited, and depends on the surface state of a corresponding mold (number of the sites interacting with the interaction 25). As an example, a ratio of a unit derived from a functional monomer in the entire constituent unit of a polymer configuring the molecularly imprinted polymer 20c includes, for example, 1 to 30 mol %, preferably 3 to 20 mol %, more preferably 5 to 10 mol %, further preferably 6 to 8 mol %.

(Other Units Derived from a Monomer)

A unit derived from a monomer that provides a group for binding a specific binding molecule (the monomer M22 comprising a group for binding a specific binding molecule and a polymerizable functional group discussed below) and/or a unit derived from a monomer that provides a group for binding a signal substance (the monomers M23₁ and M23₂ that provide a group for binding a signal substance discussed below) can also exist on the recessed part 21 surface of a polymer configuring the molecularly imprinted polymers 20, 20a and 20b. The case wherein a unit derived from a monomer that provides a group for binding a specific binding molecule exists includes the case wherein the molecularly imprinted polymers 20, 20a and 20b are particle-like. The case wherein a unit derived from a monomer that provides a group for binding a signal substance includes the case wherein the molecularly imprinted polymers 20, 20a and 20b are film-like.

### <1-3-7. Base Plate>

The material of the base plate 30 may be, for example, a material selected from the group consisting of metal, glass, and resin. The metal includes gold, silver, copper, aluminum, tungsten, molybdenum, and the like. The resin includes poly(meth)acrylate, polystyrene, ABS (acrylonitrile-butadiene-styrene copolymer), polycarbonate, polyester, polyethylene, polypropylene, nylon, polyurethane, silicone resin, fluorocarbon resin, methylpentene resin, phenol resin, melamine resin, epoxy resin, vinyl chloride resin, and the like.

The base plate 30 may be formed by combining a plurality of materials selected from the above-mentioned materials. For example, the base material 20 may be provided with a metal film on the surface of the glass or resin. In addition, it does not matter whether the shape of the base material 20 is plate-type or particle-like. A preferable example includes a gold base plate, glass base plate, gold nanoparticle, silicon dioxide particle (silica particle, glass bead, and the like), and the like.

### <1-4. Other Forms>

The base material for making a sensor for analyzing a detection subject of the present disclosure should only be configured so that a user would customize at least one of a specific binding molecule and a signal substance. Therefore, as another embodiment, a specific binding agent 42 for a target may already be bound to the groups 22′ and 22b′ for binding a specific binding molecule. In this case, a user can freely select and introduce a signal substance.

Furthermore, as another embodiment, a signal group 43 may already be bound to the groups 23′ and 23b′ for binding a signal substance. In this case, a user can freely target a detection subject, freely select a detection subject to be targeted, and can introduce a specific binding molecule for a target of a detection subject.

### <2. A Sensor for Analyzing a Detection Subject>

The sensor for analysis of the present disclosure comprises: the above-mentioned base material for making a sensor for analyzing a detection subject; and a specific binding group for a target of said detection subject bound to said group for binding a specific binding molecule, wherein said group for binding a specific binding molecule is a group that is adapted to a small substance. Alternatively, the sensor for analysis of the present disclosure comprises: the above-mentioned base material for making a sensor for analyzing a detection subject; and a specific binding group for a target of said detection subject bound to said group for binding a specific binding molecule, wherein a molecular imprint recessed part has a shape or configuration adapted to a small substance. In addition, the sensor for analysis of the present disclosure also comprises a signal group bound to a group for binding a signal substance when the above-mentioned base material for making a sensor for analyzing a detection subject has a group for binding a signal substance.

Examples of the sensor for analysis of the present disclosure are shown as analysis sensors 10, 101, 10a, 10a₁, 10b and 10c of a detection subject in FIG. 1B-1, FIG. 1B-2, FIG. 2B-1, FIG. 2B-2, FIG. 3B and FIG. 4B. As depicted, regarding the analysis sensors 10, 101, 10a, 10a₁, 10b and 10c of a detection subject, at least the specific binding group 42 for a target is bound to the groups 22′ and 22′b for binding a specific binding molecule to a target of the base materials 10′, 10a′, 10b′ and 10c′ for making an analysis sensor of a detection subject, and the signal group 43 is bound to the groups 23′ and 23b′ for binding a signal substance.

More specific examples of the sensor for analysis of the present disclosure are shown as analysis sensors 10-C, 10-P, 10-PC, 10a-C, 10a₁-C, 10a-P, 10c-P and 10b-PC of a detection subject in FIG. 5(B), FIG. 6(B), FIG. 7(B), FIG. 8(B1) and (B2), FIG. 9(B), FIG. 10(B), and FIG. 11(B). As depicted, at least the specific binding group 42 for a target is bound to the groups 22′ and 22b′ for binding a specific binding molecule to a target of the analysis sensors 10-C, 10-P, 10-PC, 10a-C, 10a-P, 10a₁-C, 10c-P and 10b-PC of a detection subject, and the signal group 43 is bound to the groups 23′ and 23b′ for binding a signal substance.

### <2-1. Detection Subject>

The detection subject of the sensor for analysis of the present disclosure comprises a target, which is not particularly limited in principle as long as the target has specific binding capacity with respect to the specific binding group 42.

Regarding the relationship between a detection subject and a target: the detection subject may be the target itself (i.e., detection subject consists of the target; hereinafter, referred to as Embodiment 1); or a part of the detection subject (e.g., the detection subject 70 discussed below) may be configured with the target (e.g., the target 72 discussed below) (hereinafter, referred to as Embodiment 2).

The chemical species of a detection subject (Embodiment 1) or a target (Embodiment 2) of a detection subject is not particularly limited, and includes, for example, a low molecular organic compound and a high molecular compound that are not derived from an organism, a low molecular organic compound and a high molecular compound that are derived from an organism. Among these chemical species, preferably a low molecular organic compound and high molecular compound that are derived from an organism are included, more preferably a low molecular organic compound and a high molecular compound derived from an animal are included, and specifically, sugar or sugar chain; lipid (e.g., phospholipid and the like); glycoprotein or glycopeptide; protein or peptide; nucleotide or polynucleotide, and the like are included.

The origin organism includes humans and animals other than a human, wherein the animals other than a human includes vertebrate animals, preferably includes mammals, which includes, for example, a mouse, rat, monkey, dog, cat, cow, horse, pig, hamster, rabbit, and goat, and the like.

The functional species of a detection subject (Embodiment 1) or a target (Embodiment 2) of a detection subject is not particularly limited, and includes plasma protein, disease marker, antigen, antibody, receptor, prion, microparticle having a film structure, and the like. In addition, when the microparticle having a film structure is set as a detection subject (Embodiment 2), the target includes: an antigen, antibody, receptor, and/or disease marker expressed on the surface of the microparticle having a film structure; an antigen, antibody, receptor and/or disease marker, wherein the microparticle having a film structure is expressed inside the film structure; and an antigen, antibody, and/or disease marker discharged by the microparticle having a film structure; and the like.

A plasma protein includes albumin, γ-globulin, fibrinogen, and the like. A disease marker includes a kidney function marker, liver function marker, inflammation marker, tumor marker, and the like, and more specifically includes urinary albumin, aspartate aminotransferase (AST), alanine aminotransferase (ALT), transferrin, ceruloplasmin, lactate dehydrogenase (LDH), alkaline phosphatase (ALP), γ-glutamyl transpeptidase (γ-GTP), C-reactive protein (CRP), α-fetoprotein (AFP), sugar chain antigen 19-9 (CA19-9) carcinoembryonic antigen (CEA), prostate-specific antigen (PSA), exosome-specific antigen (CD63, CD9, CD81, CD37, CD53, CD82, CD13, CD11, CD86, ICAM-1, Rab5, Annexin V, LAMP1, and the like).

A microparticle that has a film structure includes an extracellular vesicle, intracellular vesicle, organelle, and cell. A film structure includes a lipid bilayer structure. An extracellular vesicle includes exosome, microvesicle, apoptotic body, and the like. An intracellular vesicle includes lysosome, endosome, and the like. An organelle includes mitochondria and the like. A cell includes a cancer cell such as circulating tumor cell (CTC), other disease-associated cells, and the like. Among these microparticles that have a film structure, preferably extracellular vesicle and cell are included, more preferably exosome and cancer-associated cell are included.

A target (exosome-specific antigen or exosome antigen) expressed on the surface of an exosome includes: proteins such as CD63, CD9, CD81, CD37, CD53, CD82, CD13, CD11, CD86, ICAM-1, Rab5, Annexin V, LAMP1, EpCAM, and HER2; lipid; sugar chain; and the like.

A target (cancer cell-specific antigen) expressed on the surface of a cancer cell includes: proteins such as Caveolin-1, EpCAM, FasL, TRAIL, Galectine3, CD151, Tetraspanin 8, EGFR, HER2, RPN2, CD44, and TGF-β; lipid; sugar chain; and the like.

As discussed below, when a signal substance R43 is configured as one of the fluorescent dye pair that causes fluorescence resonance energy transfer (FRET), the target of a detection subject is coupled to the other of the fluorescent dye pair beforehand.

### <2-2. A Specific Binding Group for a Target of a Detection Subject>

In the present disclosure, the specific binding group 42 is a small substance, preferably a specific binding protein group or peptide group with a molecular weight of 45,000 or less. Molecular weight refers to calculated molecular mass.

In one embodiment, a small substance group is typically a molecule with a molecular weight that is smaller than an antibody, which is a substance that is less than 150,000, preferably a substance that is 100,000 or less, more preferably 45,000 or less, further preferably 22,000 or less, even more preferably 16,000 or less, further even more preferably a group that is less than 10,000, and any substance is used, but preferably may be a protein, polypeptide, peptide.

In the present disclosure, by selecting a group with a small molecular weight as the specific binding group 42 provided within the molecular imprint recessed part, the binding capacity per target is significantly increased (i.e., the dissociation constant Kd significantly decreases) compared to the natural binding capacity to target of said specific binding group 42 (the binding capacity in the state of not being provided to the molecular imprint recessed part, e.g., a free state or the case of being immobilized in a plane-like manner). From the viewpoint of further even more increasing such effect, the molecular weight of the specific binding group 42 includes preferably 40,000 or less, more preferably 30,000 or less, further preferably 22,000 or less, even more preferably 16,000 or less, further even more preferably 10,000 or less, especially preferably 8,000 or less.

The specific binding group 42 is not particularly limited as long as the group has specific binding capacity to a target of a detection subject and is a small substance group (provided that Fab′, Fab, and scFv are excluded). The specific binding group 42 is a group introduced by a specific binding molecule (the specific binding molecule R42 discussed below) that provides the specific binding group 42, wherein a specific example of said specific binding molecule (the specific binding molecule R42 discussed below) includes an antibody mimetic, phospholipid binding protein, and lectin, and the like. These specific binding molecules is commercially available or can be appropriately made or selected utilizing known information (e.g., library information and the like) of the subject technical field, and can be readily obtained by those skilled in the art.

A nanobody (VHH antibody) has a natural heavy chain antibody structure, and contains a single variable domain (VHH) and two constant domains (CH2 and CH3). A specific nanobody includes, but not limited to, an anti-EGFR nanobody, anti-HER2 nanobody, anti-MUC1 nanobody, anti-integrin nanobody, anti-TNF nanobody, and the like.

An antibody mimetic is any substance that can specifically bind to an antigen like an antibody but is not in structural association with an antibody, exemplified by an organic compound (not having an antibody binding domain). A specific antibody mimetic includes, but not limited to, an affibody (scaffold protein: a Z domain of a protein A, molecular weight: about 6,000, non-limited specific examples: anti-Her2 affibody, anti-EGFR affibody, anti-TNF-α affibody, anti-HSA affibody, anti-transferrin affibody, anti-fibrinogen affibody, anti-IgM affibody, and the like); aphylline (scaffold protein: γB-crystallin or ubiquitin, molecular weight: about 20,000 or about 10,000); affimer (scaffold protein: cystatin, molecular weight: about 12,000 to 14,000); afitin (scaffold protein: Sac7d, molecular weight: about 7,000); alpha body (scaffold protein: triple helix coiled coil, molecular weight: about 10,000); anticalin (scaffold protein: lipocalin, molecular weight: about 20,000, non-limited specific examples: anti-CTLA-4 anticalin, anti-VEGF anticalin, and the like); abimer (scaffold protein: film receptor domain, molecular weight: about 9,000 to 18,000); DARPins (scaffold protein: ankyrin repeat motif, molecular weight: about 10,000 to 19,000); fynomer (scaffold protein: SH3 domain of Fyn, molecular weight: about 7,000); kunitz domain peptide (scaffold protein: kunitz domain of a protease inhibitor, molecular weight: about 6,000); monobody (also referred to as adnectin; scaffold protein: 10th III-type domain of fibronectin, molecular weight: about 10,000, non-limited specific examples: anti-EGFR1 monobody, monobody of anti-HER2, and the like); nanoCLAP (scaffold protein: carbohydrate binding module 32-2 (Clostridium perfringens NagH), molecular weight: about 16,000), nanobody (e.g., a recombinant protein of a VHH antibody that a camel and alpaca have), and the like.

A phospholipid binding protein is a protein that exhibits binding activity to phospholipid. A specific example of a phospholipid binding protein includes, but not limited to, evectin 2, annexin A2, annexin V, Tim4, and the like.

Lectin is a protein that exhibits binding activity to a sugar chain. A specific example of lectin includes, but not limited to, type-C lectin, kinase-like lectin, F box lectin, type-F lectin ficolin/mannose binding lectin, galectin, X-lectin, type-L lectin, type-M lectin, type-P lectin, type-R lectin, tachylectin, sialic acid binding lectin, and the like.

A substance having specific binding capacity for a target of a detection subject other than a protein and a peptide includes, for example, the following:
Sugar: The sugar includes α-cyclodextrin (6 D-glucose coupled), β-cyclodextrin (7 D-glucose coupled), γ-cyclodextrin (8 D-glucose coupled), which are cyclic oligosaccharide wherein D-glucose is coupled by α-1,4 glycoside binding. The inside of these cyclic structures are a hydrophobic cavity. Since a molecule that matches the cavity size is recognized and enclosed by hydrophobic interaction, the molecule can be used as a molecule having specific binding capacity for a target of a detection subject;
Glycosaminoglycan with a repetition structure of disaccharide provided with a sulfate group: for example, hyaluronic acid recognizes and binds CD44 which is a film protein, thereby enabling use as a molecule having specific binding capacity to CD44. In another example, heparin exists on a cell surface, and interacts with various extracellular matrix proteins having a heparin binding domain, thereby enabling use as a molecule having specific binding capacity to a protein with a heparin binding domain. In addition, many proteoglycans wherein a sugar chain is bound to a peptide are found, thereby enabling use as a molecule having specific binding capacity to a target of a detection subject.

Lipid: lipid, e.g., phospholipid with phosphate ester, can bind various functional groups with phosphate ester, wherein phospholipid can interact with various biomolecules, thereby enabling use as a molecule having specific binding capacity to a target of a detection subject.

Nucleic acid: regarding a nucleic acid, nucleic acid aptamer is known as a nucleic acid molecule that specifically binds to a specific molecule. It can be freely manufacturing with the in vitro selection method, and there have been reports of those that specifically bind to various objects such as an organic small molecule, protein, nucleic acid cell, cell tissue, and microorganism, thereby enabling use as a molecule having specific binding capacity to a target of a detection subject.

Regarding the sensor for analysis of the present disclosure, even if the natural binding capacity to a target of a specific binding molecule (the specific binding molecule R42 discussed below) that provides the specific binding group 42 is inferior (the dissociation constant Kd is greater than an antibody) to the natural binding capacity of an antibody, the binding capacity per target would be significantly increased (i.e., the dissociation constant Kd significantly decreases) by providing the specific binding group 42 within the molecular imprint recessed parts 21 and 21c, wherein the significantly improved binding capacity may even surpass the binding capacity of an antibody per target of when introducing said antibody within the molecular imprint recessed parts 21 and 21c. In other words, according to the sensor for analysis of the present disclosure, it is possible to express sensitivity that exceeds a sensor for analysis wherein an antibody is introduced within the molecular imprint recessed part 21 and 21c even when introducing the specific binding group 42 of which binding capacity is inferior to an antibody within the molecular imprint recessed parts 21 and 21c. In view of the above, an example of a specific binding molecule (the specific binding molecule R42 discussed below) that provides the specific binding group 42 in the sensor for analysis of the present disclosure includes a molecule wherein the natural binding capacity of itself is inferior (the dissociation constant Kd is greater than an antibody) to the natural binding capacity of an antibody. However, since the sensor for analysis of the present disclosure significantly increases the binding capacity of the specific binding group 42 per target, it is possible to use a molecule wherein the natural binding capacity is increased by providing a modification that increases the binding capacity such as mutation as a specific binding molecule (the specific binding molecule R42 discussed below) that provides the specific binding group 42.

The specific binding group 42 may be a group (specific binding group having a signal group) 44 with a signal group 43 further bound like the analysis sensor 101 of a detection subject shown in FIG. 1B-2. A specific example of such specific binding group 44 that has a signal group includes: a modification protein or peptide wherein the specific modification group 42 has the signal group 43 as a modification group; a fusion protein or peptide wherein the specific binding group 42 and the signal group 43 which is a protein or a peptide are fused; and the like. As such, the natural binding capacity of the specific binding group 42 in the specific binding group 44 having a signal group is inferior to the natural binding capacity of the specific binding group 42 that does not have the signal group 43 (the specific binding group 42 in the sensors 10, 10a, 10b, and 10c for analysis of FIG. 1B-1, FIG. 2B-1, FIG. 3B, and FIG. 4B) due to the effect by the presence of the signal group 43. However, since the sensor for analysis of the present disclosure is excellent in sensitivity improvement effect, it is possible to effectively improve binding capacity and achieve excellent sensitivity improvement effect even by using a specific binding group with low natural binding capacity like the specific binding group 44 that has a signal group. In the present disclosure, while it is possible to carry out analysis with excellent sensitivity (low dissociation constant) even when using the group 44 having the specific binding group 42 and the signal group 43 like the analysis sensor 101 of a detection subject shown in FIG. 1B-2, from the viewpoint of improving reproducibility and/or lowering the detection limit value, it is preferable that the signal group 43 and the specific binding group 42 are spaced apart in the surface direction of the recessed part of the molecular imprint as shown in FIG. 2B, or have the functional group 24b (see FIG. 3A) therebetween as shown in FIG. 3B, rather than being bound to one another.

Furthermore, the specific binding group 42 may be a group (specific binding group having a different signal group) 42₁ wherein a different signal group sg is further bound like the analysis sensor 10a₁ of a detection subject shown in FIG. 2B-2. While the signal group 43 bound to the specific binding group 42 shown in FIG. 1B-2 above is used for sensing of the analysis sensor 101 of a detection subject, the different signal group sg bound to this specific binding group 42 shown in FIG. 2B-2 are different in that they are not used for sensing of the analysis sensor 10a₁ of a detection subject. Since the molecular weight of the specific binding group 42 is small, the different signal group sg is merely provided from the viewpoint of making the handling of the specific binding molecule (the specific binding molecule R42 discussed below) that provides the specific binding group 42 easy, wherein not the different signal group sg but the signal group 43 is used for the sensing of the analysis sensor 10a₁ of a detection subject shown in FIG. 2B-2.

A specific example of such specific binding group 42₁ having a different signal group includes: a modification protein or peptide wherein the specific binding group 42 has the different signal group sg as a modification group; a fusion protein or peptide wherein the specific binding group 42 and the different signal group sg which is a protein or a peptide are fused; and the like. A such, the natural binding capacity of the specific binding group 42 in the specific binding group 42₁ having a different signal group is inferior to the natural binding capacity of the specific binding group 42 that does not have the different signal sg (e.g., the specific binding group 42 in the sensors 10, 10a, 10b, and 10c for analysis of FIG. 1B-1, FIG. 2B-1, FIG. 3B, and FIG. 4B) due to the effect of the existence of the different signal group sg. However, since the sensor for analysis of the present disclosure is excellent in sensitivity improvement effect, it is possible to effectively improve the binding capacity and achieve excellent sensitivity improvement effect even when using a specific binding group with low natural binding capacity like the specific binding group 42₁ having a different signal group.

### <2-3. Signal Group>

The signal group 43 functions as a group that reads binding information between a target of a detection subject and the specific binding group 42 for said target. The signal group 43 is not particularly limited as long as the detected signal intensity changes and the spectrum changes (e.g., the peak shifts) due to the binding of a target in the molecular imprint recessed parts 21 and 21c. For example, a signal substance that provides the signal group 43 (the signal substance R43 discussed below) includes a fluorescent substance, radioactive element-containing substance, magnetic substance, and the like. From the viewpoint of easiness of detection and the like, it is preferable that the signal substance is a fluorescent substance. A fluorescent substance includes: fluorescent dye such as fluorescein dye, cyanine dye such as indocyanine dye (DY647, Cy5, DY490, and the like), rhodamine dye (CR110 and TAMRA), coumarin dye, EvoBlue dye, oxazine dye (ATTO), carbopyronin dye, and Alexa dye; fluorescent protein such as GFP; nanoparticle such as gold colloid and quantum dot; and the like. A radioactive element-containing substance includes sugar, amino acid, and nucleic acid labeled with a radioactive isotope such as ¹⁸F, MRI probe labeled with ¹⁹F, and the like. A magnetic substance includes a matter having a magnetic body such as ferrichrome, ferrite nanoparticle, nanomagnetic particle, and the like. Among the above-mentioned signal substances, rhodamine dye is preferable from the viewpoint of further improving detection limitation and/or sensitivity.

In addition, the signal group 43 can be configured as one of the fluorescent dye pair that causes fluorescence resonance energy transfer (FRET). The fluorescent dye pair that causes FRET is not particularly limited, and is also not limited in terms of which of the donor dye and accepter dye is selected as the signal group 43. Preferably, donor dye can be selected as the signal group 43. A specific example of the combination of donor dye/accepter dye configuring the pair of fluorescent dyes causing FRET includes fluorescein isothiocyanate (FITC)/tetramethylrhodamine isothiocyanate (TRITC), Alexa Fluor647/Cy5.5, HiLyte Fluor647/Cy5.5, R-phycoerythrin (R-PE)/allophycocyanin (APC), and the like.

### <2-4. Usage>

The sensor for analysis of the present disclosure is used for sensing of a detection subject. While a more specific usage is determined in accordance with the type of the specific binding group, the sensor for analysis of the present disclosure can be used, for example, for the purpose of diagnosis, or the purpose of therapy monitoring or the like based on kidney function, liver function, the presence/absence or degree of inflammation; presence/absence or degree of tumor, or the like.

In addition, the sensor for analysis of the present disclosure may be formulated to be liquid-like or solid-like (e.g., powder-like) as a sensing reagent in the case of the form wherein a molecularly imprinted polymer is a particle and is not immobilized on a base plate. The sensing reagent comprises another pharmacologically accepted component. A solid body and/or liquid that does not affect the stability and recognition capacity of a sensing reagent is appropriately selected as the another component, which includes, for example, water, salt such as NaCl, buffering agent, stabilizing agent, antioxidizing agent, antiseptic agent, pH regulating agent, surface active agent, diluting agent, binding agent, lubricating agent, and the like.

A reagent for sensing can be obtained by formulation based on a normal formulation method using the sensor for analysis of the present disclosure and the above-mentioned another component.

Other than those mentioned above, an example of the usage of the sensing reagent includes in vitro imaging and in vivo imaging utilizing the feature of a particle not being immobilized on a base plate.

An example of the in vitro imaging includes a method of making a sensing reagent corresponding to a detection subject in a cell or on the surface and adding the sensing reagent to said cell to carry out imaging of the kinetics of the biomolecule in the cell or on the surface with a signal detection means such as a fluorescence microscope.

An example of the in vivo imaging includes a method of making a sensing reagent corresponding to the detection subject existing in a body, administering the sensing reagent to an experimentation animal, a circulatory organ of an animal such as a human, e.g., blood, and carrying out imaging of the biokinetics of a detection subject with a signal detection means. The sensing reagent used for the in vivo imaging preferably configures the sensor 10c-P for analysis shown in FIG. 10(B) as an effective component when the detection subject is something other than a plasma protein (especially albumin). In this case, the sensor 10c-P for analysis is a sensor wherein the molecular imprint recessed part 21c molds plasma protein (especially albumin) as a mold and the interaction group 25 is designed as a group that specifically interacts with the surface of said plasma protein (especially albumin). Such sensing reagent used for the in vivo imaging can be injected before surgery and be used as a navigation drug that carries out in vivo imaging of a tumor during the surgery when a detection subject is configured as a tumor marker.

### <3. Manufacturing Method of a Base Material for Making a Sensor for Analyzing a Detection Subject>

The manufacturing method of the base material for making a sensor for analyzing a detection subject of the present disclosure can use a method of synthesizing a molecularly imprinted polymer and introducing a predetermined functional group within a molecular imprint recessed part without a particular limitation. Several examples of the manufacturing method of the base material for making a sensor for analyzing a detection subject of the present disclosure include the following.

<3-1. Manufacturing Method of a Base Material for Making a Sensor for Analyzing a Detection Subject that is Base Plate-Type>

For example, the manufacturing method of the base material 10-C′ for making a sensor for analyzing a detection subject that is base plate-type which is shown in FIG. 5(A) comprises the following step (1-1) to step (1-4):
the monolayer formation step (1-1) of forming a monolayer having a group for binding a specific binding molecule for a target of a detection subject and a polymerization initiating group on a surface at the base plate;
the step (1-2) of introducing a mold to said group for binding a specific binding molecule via a first reversible linking group;
the step (1-3) of forming a polymer film on said base plate surface by adding a polymerizable monomer, using said polymerizable monomer as a substrate, using said polymerization initiating group as a polymerizable initiating agent, and synthesizing a molecularly imprinted polymer for a part of said surface of said mold; and
the step (1-4) of cleaving said first reversible linking group for conversion to said group for binding a specific binding molecule as well as removal of said mold.

Provided that said group for binding a specific binding molecule is a group adapted to a small substance.

The following limitations are further added to the manufacturing method of the base material 10a-C′ for making a sensor for analyzing a detection subject that is base plate-type which is shown in FIG. 8(a), further comprising the group 23′ for binding a signal substance in the base material 10-C′ for making a sensor for analyzing a detection subject that is base plate-type which is shown in FIG. 5(A).
Comprising the modification step of modifying a surface of said mold with a polymerizable functional group via a second reversible linking group before said step (1-3);
using said polymerizable monomer and said polymerizable functional group as a substrate, using said polymerization initiating group as a polymerizable initiating agent, and synthesizing a molecularly imprinted polymer for a part of said surface of said mold in said step (1-3);
cleaving said first reversible linking group and said second reversible linking group for conversion to said group for binding a specific binding molecule and a group for binding a signal substance, respectively, as well as removal of said mold in said step (1-4).

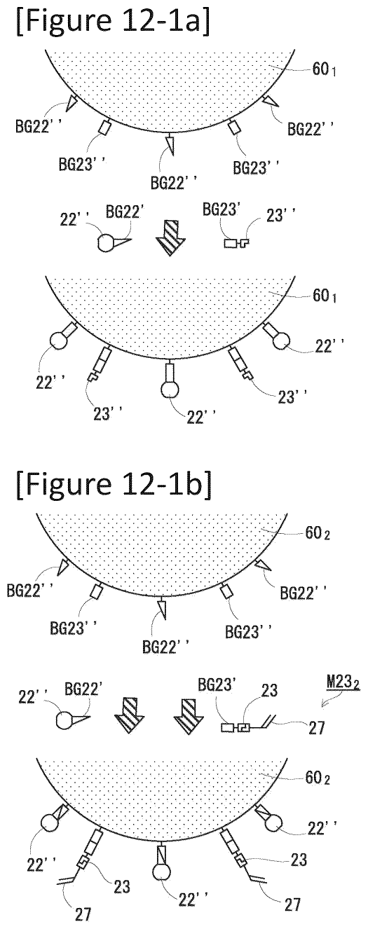

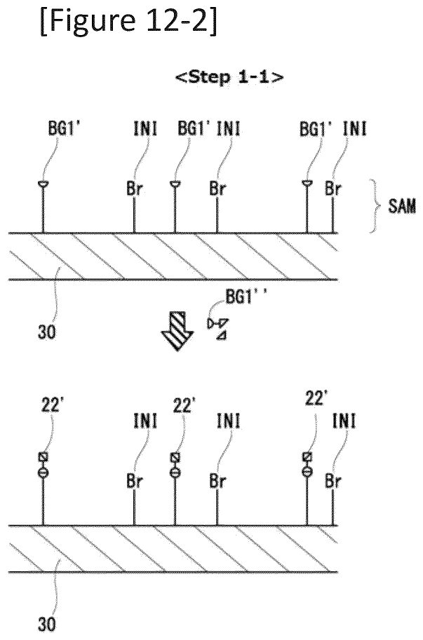

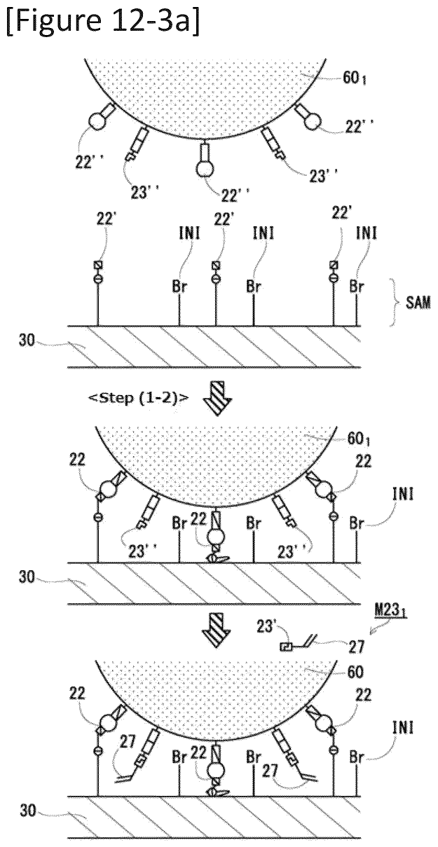

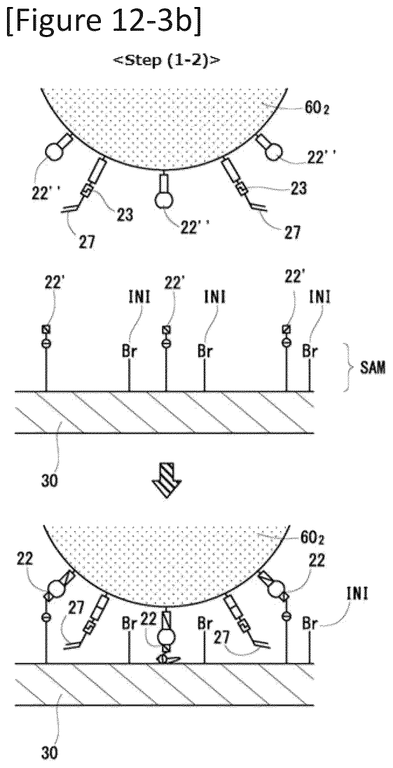

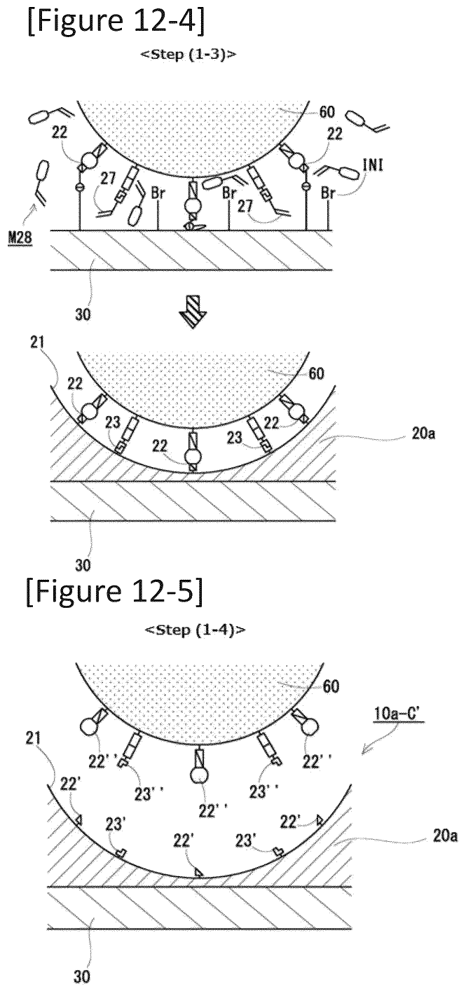

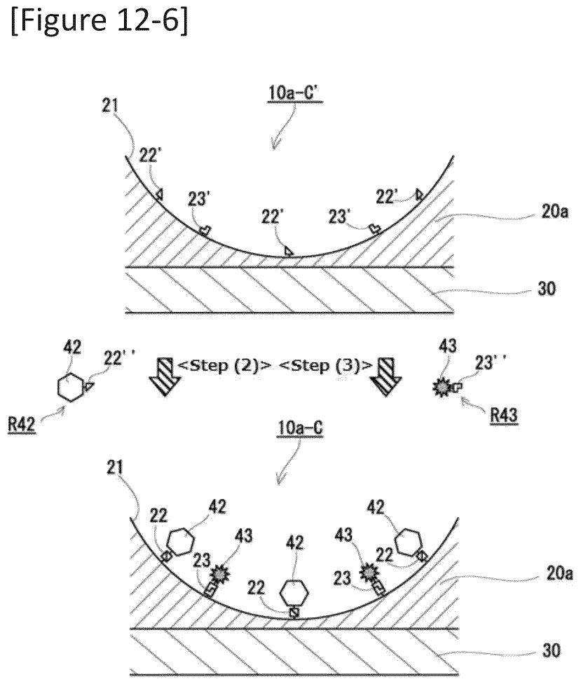

FIG. 12-1 to FIG. 12-5 schematically show the manufacturing method of the base material 10a-C′ for making a sensor for analyzing a detection subject that is base plate-type which is shown in FIG. 8(A). Specifically, FIG. 12-1 (FIG. 12-la and FIG. 12-2b), FIG. 12-2, FIG. 12-3, FIG. 12-4 and FIG. 12-5 schematically show the step of preparing a mold, step (1-1), step (1-2), step (1-3) and step (1-4), respectively. In other words, the following steps are comprised in a method of manufacturing the base material 10a-C′ for making a sensor for analyzing a detection subject in the depicted embodiment.

### (I)

The monolayer formation step (1-1) of forming a monolayer SAM having the group 22′ for binding a specific binding molecule to a target of a detection subject and a polymerization initiating group INI on a surface at the base plate 30;
the step (1-2) of introducing a mold 60 to the group 22′ for binding a specific binding molecule via the first reversible linking group 22;
the step (1-3) of forming a polymer film on the base plate 30 surface by adding a polymerizable monomer M28, using the polymerizable monomer M28 as a substrate, using the polymerization initiating group INI as a polymerizable initiating agent, and synthesizing the molecularly imprinted polymer 20a for a part of a surface of the mold 60;
the step (1-4) of cleaving the first reversible linking group 22 for conversion to the group 22′ for binding a specific binding molecule as well as removal of the mold 60; provided,

### (II)

comprising the modification step of modifying a surface of the mold 60 with the polymerizable functional group 27 via the second reversible linking group 23;
using the polymerizable monomer M28 and polymerizable functional group 27 as a substrate, using the polymerization initiating group INI as a polymerizable initiating agent, and synthesizing a molecularly imprinted polymer 20a for a part of said surface of the mold 60 in said step (1-3);
cleaving the first reversible linking group 22 and said second reversible linking group 23 for conversion to group 22′ for binding a specific binding molecule and the group 23′ for binding a signal substance, respectively, as well as removal of the mold 60 in said step (1-4).

Each step explained in detail below while referring to the drawings. The manufacturing method of the base material 10-C′ for making a sensor for analyzing a detection subject that is base plate-type which is shown in FIG. 5(A) would be formed when the above-mentioned limitation (II) is removed from the manufacturing method of the base material 10a-C′ for making a sensor for analyzing a detection subject that is base plate-type discussed in detail below.

### <3-1-1. The Step of Preparing a Mold>

The mold used in the manufacturing method of the base material 10a-C′ for making a sensor for analyzing a detection subject that is base plate-type which is shown in FIG. 12-2 to FIG. 12-5 below is prepared as a mold wherein appropriate surface modification has been made as needed.

### (Mold)

The mold 60 may be the same substance as a detection subject or a target of the detection subject, or may be a substance that is different from a detection subject or a target of the detection subject. In addition, the mold 60 in the case of being a substance different from a detection subject or a target of the detection subject may be a biomolecule, or may be a particle core (also referred to as artificial particle).

A biomolecule preferably includes a plasma protein (albumin, γ-globulin, fibrinogen, and the like). In addition, the molecular weight of the biomolecule includes, for example, 1.8 to 15-fold, preferably 3 to 14-fold, more preferably 4 to 13.5-fold, 7 to 13-fold, 8 to 12.5-fold, or 10 to 12-fold of the molecular weight of the specific binding group 42. A more preferable biomolecule includes albumin.

A particle core is not particularly limited to the extent of being able to be used as a mold in a molecular imprint, including artificially manufactured inorganic particle and organic particle. An inorganic particle includes metal, oxide of metal, nitride, fluoride, sulfide, boride and a composite compound thereof, as well as hydroxyapatite, and the like, preferably silicon dioxide (silica). In addition, an organic particle includes latex cured product, dextran, chitosan, polylactic acid, poly(meth)acrylate, polystyrene, polyethyleneimine, and the like.

A particle core is preferably used as the mold 60 in the embodiment shown in FIG. 12-2 to FIG. 12-5. Since a particle core is an industrial product that is particle-size controlled, it is easy to control and homogenize the size of the recess part formed into the base material for making a sensor for analysis, which is preferable in terms of being able to make a sensor for analysis with even more excellent analysis property from the obtained base material for making a sensor for analysis. In addition, it is possible to use a particle core of which surface has been modified so as to have a desired binding property at the surface.

Since the size of the molecular imprint recessed part 21 depends on the size of the mold 60, the size of the mold 60 can be appropriately determined in accordance with the size of a detection subject or a target of the detection subject. It is enough to use the mold 60 having about the same or greater size as said detection subject or a target of the detection subject in order to form a recessed part 21 for receiving a detection subject or a target of the detection subject of interest. For example, the average particle size of the mold 60 includes, for example, 1 nm to 10 μm, preferably 50 to 1 μm, more preferably 100 to 500 nm, further preferably 150 to 250 nm, even more preferably 180 to 220 nm. Alternatively, when the average particle size of the mold 60 is R (nm) and the molecular weight of the specific binding group 42 is M, the average particle size of the mold 60 includes a particle size wherein the parameter R/M satisfies, for example, 0.005 to 0.05, preferably 0.01 to 0.04, more preferably 0.012 to 0.035, 0.013 to 0.035, 0.02 to 0.035, or 0.03 to 0.034. Average particle size refers to the Z average particle size measured by the dynamic light scattering method.

### (Surface Modification of a Mold)

The mold 60 can be prepared in the form of which surface is modified by the binding group 22″ as shown as the mold 601 and the mold 602 in FIG. 12-la and FIG. 12-1b.

A method of preparing such a mold includes an embodiment wherein a surface of the mold 601 is modified with the binding group 23″ in addition to the binding group 22″ as shown in FIG. 12-1a, a method of modifying a surface of the mold 602 with a polymerizable functional group via a reversible linking group 23 in addition to the binding group 22″ as shown in FIG. 12-1b, and the like.

For example, in the example of FIG. 12-1a, the mold 601 having the binding group BG22″ and the binding group BG23″ on the surface is used to appropriately react a molecule comprising the binding group BG22′ corresponding to the binding group BG22″ and the binding group 22″ and a molecule comprising the binding group BG23′ corresponding to the binding group BG23″ and the binding group 23″, whereby obtaining the mold 601 modified with the binding group 23″ in addition to the binding group 22″ on the surface.

In FIG. 12-1a, the binding group BG22″ is not particularly limited, but includes, for example, the groups shown in column 2-1 of Table 2 above, preferably includes a carboxyl group. The binding group BG22′ is not particularly limited, which includes, for example, the groups shown in column 2-2 of Table 2 above, preferably includes an amino group as the binding group BG22′. The binding group 22″ is not particularly limited (provided the group is different from the binding group BG22′), which includes the groups shown in column 1-2 of Table 1 above, preferably includes a polyhistidine tag. In addition, the binding group BG 23″ is not particularly limited, but includes, for example, the groups shown in column 2-1 of Table 2 above, preferably includes a carboxyl group. The binding group BG23′ is not particularly limited, which includes the groups shown in column 2-2 of Table 2 above, preferably includes an amino group as the binding group BG23′. The binding group 23″ is not particularly limited (provided the group is different from the binding group 22′), which includes the groups shown in column 1-2 of Table 1 above, preferably includes thiol group.

In addition, in the example of FIG. 12-1b, the mold 602 having the binding group BG22″ and the binding group BG23″ on the surface is used to appropriately react a molecule comprising the binding group BG22′ corresponding to the binding group BG22″ and the binding group 22″ and the polymerizable monomer M23₂ comprising the binding group BG23′ corresponding to the binding group BG23″, the second reversible linking group 23 and the polymerizable functional group 27, and obtain a mold 602 having the polymerizable functional group 27 and a second reversible linking group 23 as well as the binding group 22″ that can form the first reversible linking group 22. In the example of FIG. 12-1b, the modification step of modifying the surface of the mold 60 with the polymerizable functional group 27 via the second reversible linking group 23 is comprised in the step of preparing a mold.

In FIG. 12-1b, the binding group BG22″ is not particularly limited, but includes, for example, the groups shown in column 2-1 of Table 2 above, preferably includes a carboxyl group. The binding group BG22′ is not particularly limited, which includes, for example, the groups shown in column 2-2 of Table 2 above, preferably includes an amino group as the binding group BG22′. In addition, the binding group 22″ is not particularly limited (provided the group is different from the binding group BG22′), which includes the groups shown in column 1-2 of Table 1 above, preferably includes a polyhistidine tag. The binding group BG23″ is not particularly limited, which includes, for example, the groups shown in column 2-1 of Table 1 above, preferably includes an amino group. The binding group BG23′ in the polymerizable monomer M23₂ is not particularly limited, which includes, for example, the groups shown in column 2-2 of Table 1 above, preferably includes a carboxylic acid-active ester group. The second reversible linking group 23 is not particularly limited, which includes, for example, the groups shown in column 1-3 of Table 1 above, preferably includes a disulfide bond.

In FIG. 12-1b, the polymerizable functional group 27 only needs to contain a vinyl group, which preferably includes a (meth)acryloyl group.

As shown in FIG. 12-1b, the second reversible linking group 23 can be delivered to the surface of the mold 60 by using the mold 602 having the binding group BG23″ on the surface beforehand.

Accordingly, it is possible to obtain the molds 601 and 602 (hereinafter, may also be collectively referred to as the “mold 60”) wherein the surface is modified with the binding group 22″ (and the binding group 23″ or the polymerizable functional group 27 via the second reversible linking group as needed).

Furthermore, depending on the substance employed as the mold 60, there is a substance that naturally has the binding group 22″ (e.g., there is a case of selecting a sugar protein as a mold, the case of being able to use a sugar group that a sugar protein naturally has as the binding group 22″, and the like). In such a case, the mold 60 does not require the above-mentioned step.

### <3-1-2. Step (1-1)>

In step (1-1), as shown in FIG. 12-2, a monolayer SAM having the group 22′ for binding a specific binding molecule to a target of a detection subject and the polymerization initiating group INI on the surface is formed at the base plate 30.

In the depicted example, specifically, a monolayer SAM having the polymerization initiating group INI and the binding group BG1′ on the surface is first formed on the base material 30 surface. This monolayer can be formed as a mixed self-assembled monolayer (mixed SAMs) by mixed self-assembling using a molecule having the polymerization initiating group INI at the terminal and a molecule having the binding group BG1′ that is different from the polymerization initiating group INI at the terminal.

The polymerization initiating group INI is not particularly limited as long as it has a structure that may function as a polymerization initiating agent, which can be appropriately determined by those skilled in the art in accordance with the polymerization reaction used in the polymerization step discussed below. For example, the polymerization initiating group INI includes a group having a structure that generates radical upon polymerization reaction, specifically, a carbon-halogen binding group derived from organic halogen (—CX group; X shows halogen atom), an azo group derived from an azo compound (—R—N═N—R′— group), a group for RAFT polymerization derived from a RAFT agent (e.g., a thiocarbonylthio group, and the like), and a group derived from TEMPO. The depicted embodiment exemplifies the case in which the polymerization initiating group INI is a —CBr group.

A group that can extend the group 22′ for binding a specific binding molecule to a target of a detection subject is appropriately selected as the binding group BG1′. For example, the groups shown in column 2-1 of Table 2 above are included, preferably an amino group is included. The group 22′ for binding a specific binding molecule is further introduced to the surface of the monolayer SAM by binding a substance for extending the group 22′ for binding a specific binding molecule to the binding group BG1′. For example, a substance for extending the group 22′ for binding a specific binding molecule includes a molecule having a group that can form the group 22′ for binding a specific binding molecule and the binding group BG1″ that corresponds to the binding group BG1′, or a molecule having the group 22′ for binding a specific binding molecule and the binding group BG1″ that corresponds to the binding group BG1′. The binding group BG1″ that corresponds to the binding group BG1′ includes, for example, the groups shown in column 2-2 of Table 2 above. In the depicted embodiment, as an example of a molecule having a group that can form the group 22′ for binding a specific binding molecule and the binding group BG1″ that corresponds to the binding group BG1′, nitrilotriacetic acid (NTA) which is an aminopolycarboxylic acid-based chelating agent is used to bind NTA to introduce a group derived from NTA and nickel ion is further coordinated to a group derived from NTA to form a group derived from nickel-nitrilotriacetic acid which is an unlimited example of the group 22′ for binding a specific binding molecule.

Furthermore, the example of FIG. 12-2 shows an embodiment of gradually forming a monolayer SAM having the group 22′ for binding a specific binding molecule and the polymerization initiating group INI on the surface by first making a mixed self-assembled monolayer having the binding group BG1′ on the surface and then extending the binding group BG22′ to the binding group BG1′, but the present disclosure is not limited to this embodiment. For example, it is possible to gradually form a monolayer SAM having the group 22′ for binding a specific binding molecule and the polymerization initiating group INI on the surface by forming a mixed self-assembled monolayer (mixed SAMs) by mixed self-assembling using a molecule having the polymerizable initiating group INI at a terminal and a molecule having the binding group BG22′ at a terminal on the base material 30 surface.

Accordingly, a monolayer SAM having the group 22′ for binding a specific binding molecule and the polymerization initiating group INI on the surface is formed at the base plate 30.

### <3-1-3. Step (1-2)>

In step (1-2), as shown in FIG. 12-3a and FIG. 12-3b, a mold 60 is introduced to the group 22′ for binding a specific binding molecule on the base plate 30 via the first reversible linking group 22.

The first reversible linking group 22 is formed by binding between the group 22′ for binding a specific binding molecule on the base plate 30 and the binding group 22′ on the mold 60, which includes, for example, the groups shown in column 1-3 of Table 1 above, preferably includes a coordinate bond.

The example shown in FIG. 12-3a shows the case of using the mold 601 obtained in FIG. 12-1a, and the example shown in FIG. 12-3b shows the case of using the mold 602 obtained in FIG. 12-1b.

In the example shown in FIG. 12-3a, the surface of the mold 601 immobilized on the base plate 30 can be modified by introducing the mold 60 in the state in which the polymerizable functional group 27 is still not introduced to the group 22′ for binding a specific binding molecule on the base plate 30 via the first reversible linking group 22, and then using the polymerizable monomer M23₁ having the group 23′ for binding a signal substance and the polymerizable functional group 27 in a state in which the mold 60 is immobilized on the base plate 30. In the example of FIG. 12-3a, the modification step of modifying the surface of the mold 60 with the polymerizable functional group 27 via the second reversible linking group 23 is comprised after the step of introducing a mold.

The group 23′ for binding a signal substance in the polymerizable monomer M23₁ is as discussed in “1-3-3. A group for binding a signal substance”, which preferably includes a pyridyl disulfide group. The polymerizable function group 27 should only contain a vinyl group, which preferably includes a (meth)acryloyl group.

### <3-1-4. Step (1-3)>

In step (1-3), as shown in FIG. 12-4, a polymer film is formed on the base plate 30 surface by adding the polymerizable monomer M28, using the polymerizable monomer M28 and the polymerizable functional group 27 as a substrate, using the polymerization initiating group INI as a polymerizable initiating agent, and synthesizing the molecularly imprinted polymer 20a for a part of the surface of the mold 60.

In the depicted example, only one type of polymerizable monomer is shown, but a plurality of types may be combined as the polymerizable monomer. In addition, the polymerizable monomer is as discussed in the “1-3-6. A polymer configuring a molecularly imprinted polymer” above. The polymerizable monomer M28 used in the depicted example is preferably a biocompatible monomer, wherein the biocompatible monomer is as discussed in “(A unit derived from a biocompatible monomer)” of “1-3-6. A polymer configuring a molecularly imprinted polymer” above.

Surface-initiated atom transfer radical polymerization (SI-ATRP) progresses by constructing on the base material 30 surface a polymerization reaction system where the polymerizable functional group 27, polymerizable monomer M28, polymerizable starting group INI and mold 60 coexist. Said polymerization reaction system further preferably comprises a transition metal complex formed of a transition metal or a transition metal compound and a ligand as a polymerization catalyst, and preferably a reducing agent is further used.

A transition metal or transition metal compound includes metal copper or copper compound, and the copper compound includes chloride, bromide, iodide, cyanide, oxide, hydroxide, acetate, sulfate, nitrified product, preferably bromide. A ligand is preferably multidentate amine, specifically including bidentate to sexadentate ligands. Among the above, a bidentate ligand is preferably included, more preferably 2,2-bipyridyl, 4,4′-di-(5-nonyl)-2,2′-bipyridyl, N-(n-propyl)pyridylmethanimine, N-(n-octyl)pyridylmethanimine, and the like are included, more preferably 2,2-bipyridyl is included.

A reducing agent includes alcohol, aldehyde, phenols, and organic compound, and the like, preferably includes an organic acid compound. An organic acid compound includes citric acid, oxalic acid, ascorbic acid, ascorbate, ascorbic acid ester, and the like, preferably includes ascorbic acid, ascorbate, ascorbic acid ester, and the like, more preferably includes ascorbic acid.

A water-based solvent such as a buffering liquid is used as a solvent, which preferably includes phosphate buffer liquid. In addition, a dissolving agent preferably comprises NaCl, wherein the concentration of NaCl includes, for example, 100 to 200 mM, preferably 120 to 160 mM. In addition, the pH of the dissolving agent includes, for example, 6 to 8, preferably 6.8 to 7.8, more preferably 7.2 to 7.6.

When a polymer chain elongates from a polymerization initiating group INI which is a radical generation source with the polymerizable monomer M28 as a substrate, the thickness of the polymer film is increased and the elongated polymer chain reaches the mold 60 surface, the modified polymerizable functional group 27 is also taken in as a substrate at said surface to synthesize the molecularly imprinted polymer 20a so as to form the recessed part 21 in the shape along the surface shape of the mold 60. The polymer film of the molecularly imprinted polymer 20a can grow to a thickness corresponding to about ⅓ to ½ of the upper and lower diameters (when the upper side of the drawing is considered up) of the mold 60 introduced to the base material 30. This causes obtainment of a polymer film of the molecularly imprinted polymer 20a.

### <3-1-5. Step (1-4)>

In step (1-4), as shown in FIG. 12-5, the mold 60 is removed by cleaving the first reversible linking group 22 for conversion to the group 22′ for binding a specific binding molecule to a target, and cleaving the second reversible linking group 23 for conversion to the group 23′ for binding a signal substance. Since the second reversible linking group 23 is delivered only to the surface of the mold 60 in the above-mentioned surface modification step, the group 22′ for binding a specific binding molecule remains in the molecular imprint recessed part 21 which is a trace of removal of the mold 60, and the group 23′ for binding a signal substance occurred from the second reversible linking group 23 is disposed only within the recessed part 21. This causes obtainment of the base material 10a-C′ for making a sensor for analysis.

<3-2. Manufacturing Method of a Base Material for Making a Sensor for Analyzing a Detection Subject that is Particle-Like-1>

For example, the manufacturing method of the base material 10-P′ for making a sensor for analyzing a detection subject that is base plate-type which is shown in FIG. 6(A) comprises the following step (1-11) to step (1-13):
the step (1-11) of preparing a mold of a molecular imprint wherein the surface is modified with a polymerizable functional group via a first reversible linking group;
the step (1-12) of using said polymerizable functional group as a substrate and synthesizing a molecularly imprinted polymer for a part of said surface of said mold; and
the step (1-13) of cleaving said first reversible linking group and removing said mold from said molecularly imprinted polymer to generate said group for binding a specific binding molecule.

Provided that said group for binding a specific binding molecule is a group adapted to a small substance.

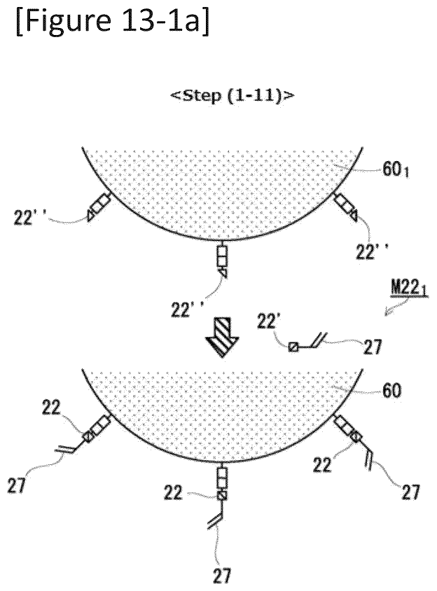

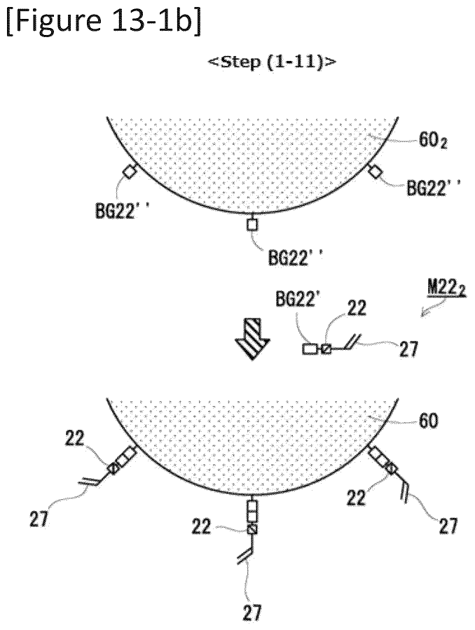

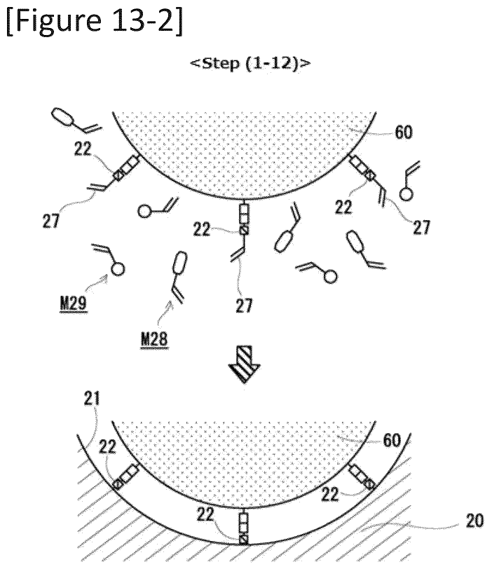

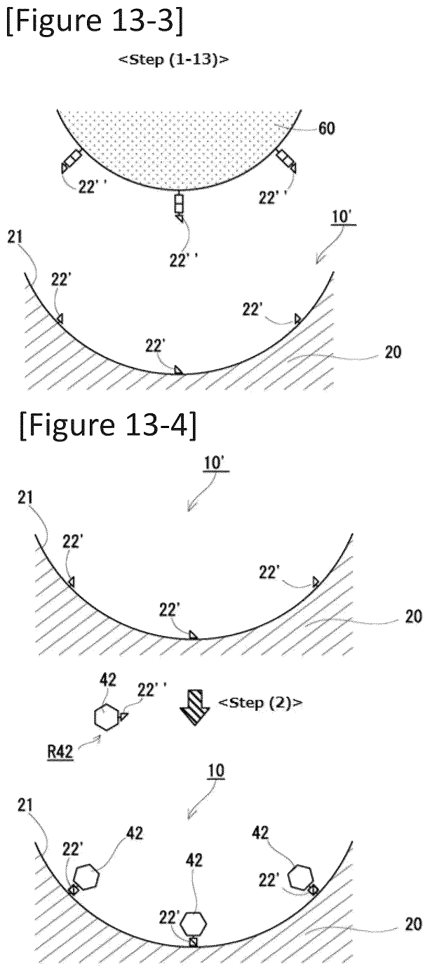

FIG. 13-1 to FIG. 13-3 schematically show a manufacturing method of the base material 10-P′ for making a sensor for analyzing a detection subject that is base plate-type which is shown in FIG. 6(A). Specifically, FIG. 13-1, FIG. 13-2, and FIG. 13-3 schematically show step (1-11), step (1-12), and step (1-13), respectively. In other words, in the depicted embodiment, the method of manufacturing the base material 10-P′ for making a sensor for analyzing a detection subject comprises the following steps:
the step (1-11) of preparing the mold 60 of a molecular imprint wherein the surface is modified with the polymerizable functional group 27 via the first reversible linking group 22;
the step (1-12) of using the polymerizable functional group 27 as a substrate to synthesize the molecularly imprinted polymer 20 for a part of the surface of the mold 60; and
the step (1-13) of cleaving the first reversible linking group 22 to remove the mold 60 from the molecularly imprinted polymer 20 and cause the group 22′ for binding a specific binding molecule.

Each step is discussed in detail below while referring to the drawings.

### <3-2-1. Step (1-11)>

In step (1-11), as shown in FIG. 13-1 (FIG. 13-la and FIG. 13-1b), a mold 60 of a molecular imprint, wherein the surface is modified with a polymerizable functional group 27 via the first reversible linking group 22, is prepared.

In FIG. 13-1a, the surface of the mold 601 of the molecular imprint, wherein the surface is modified with the binding group 22″, causes the monomer M22₁ comprising the group 22′ for binding a specific binding molecule to a target of a detection subject and the polymerizable functional group 27 to act, to carry out modification with the polymerizable functional group 27 via the first reversible linking group 22 formed by the reaction between the binding group 22″ and the group 22′ for binding a specific binding molecule.

In FIG. 13-1a, the binding group 22″ is not particularly limited, but includes, for example, the groups shown in column 1-2 of Table 1 above, preferably includes a thiol group. The group 22′ for binding in the polymerizable monomer M22₁ is not particularly limited, and includes, for example, a group shown in column 1-1 of Table 1 above, preferably includes a pyridyl disulfide group. The first reversible linking group 22 is not particularly limited, which includes, for example, the groups shown in column 1-3 of Table 1 above, preferably includes a disulfide bond.

In addition, in the example of FIG. 13-1b, the mold 602 having the binding group BG22″ at the surface is used to react the polymerizable monomer M222 comprising the binding group BG22′ that corresponds to the binding group BG22″, the first reversible linking group 22, and the polymerizable functional group 27 to obtain the mold 602 modified with the binding group 22″ via the first reversible linking group 22.

In FIG. 13-1b, the binding group BG22″ is not particularly limited, but includes, for example, the groups shown in column 2-1 of Table 2 above, preferably includes an amino group. The binding group BG22′ in the polymerizable monomer M222 is not particularly limited, which includes, for example, the groups shown in column 2-2 of Table 2 above, preferably includes a carboxylic acid-active ester group as the binding group BG22′. In addition, the first reversible linking group 22 is not particularly limited, which includes, for example, the groups shown in column 1-3 of Table 1 above, preferably includes a disulfide bond.

In FIG. 13-la and FIG. 13-1b, the polymerizable functional group 27 should only contain a vinyl group, preferably includes a (meth)acryloyl group.

In FIG. 13-la and FIG. 13-1b, the mold 601 and the mold 602 (hereinafter, collectively described as the “mold 60”) are as discussed in “(Mold)” of “3-1-2. Mold and modification step” above.

As such, the mold 60 modified with the polymerizable functional group 27 via the first reversible linking group 22 is obtained at the surface.

### <3-2-2. Step (1-12)>

In step (1-12), as shown in FIG. 13-2, the polymerizable functional group 27 is used as a substrate to synthesize the molecularly imprinted polymer 20 for a part of the surface of the mold 60.

In the depicted embodiment, an example using M28 and M29 as a polymerizable monomer is shown, but three or more types may be combined as the polymerizable monomer. In addition, the polymerizable monomer is as discussed in “1-3-6. A polymer configuring a molecularly imprinted polymer” above. The polymerizable monomer M28 used in the depicted example is a biocompatible monomer, wherein the biocompatible monomer is as discussed in “(A unit derived from a biocompatible monomer)” of “1-3-6. A polymer configuring a molecularly imprinted polymer” above. The polymerizable monomer M29 used in the depicted example is N-substituted or unsubstituted (meth)acrylamide, wherein the N-substituted or unsubstituted (meth)acrylamide is as discussed in the “(A unit derived from N-substituted or unsubstituted (meth)acrylamide)” of “1-3-6. A polymer configuring a molecularly imprinted polymer” above.

The concentration of the mold 60 in a polymerization reaction liquid is not particularly limited, but includes, for example, 1 to 10 μmol/L, preferably 3 to 8 μmol/L, more preferably 4.5 to 7 μmol/L, further preferably 5.5 to 6.5 μmol/L.

The concentration of the polymerizable monomer M28 in a polymerization reaction liquid is not particularly limited, but includes, for example, 1 to 10 mmol/L, preferably 3 to 8 mmol/L, more preferably, 4.5 to 7 mmol/L, further preferably 5.5 to 6.5 mmol/L. In addition, the amount of use of the polymerizable monomer M28 with respect to the mold 60 includes, as the amount of use of the polymerizable monomer M28 per 1 μmol of the mold 60, for example, 0.1 to 2 mmol, preferably 0.3 to 1.8 mmol, more preferably 0.5 to 1.5 mmol, further preferably 0.8 to 1.2 mmol.

The concentration of the polymerizable monomer M29 in a polymerization reaction liquid is not particularly limited, but includes, for example, 50 to 200 mmol/L, preferably 80 to 130 mmol/L, more preferably 100 to 115 mmol/L. In addition, the amount of use of the polymerizable monomer M29 with respect to the mold 60 includes, as the amount of use of the polymerizable monomer M29 per 1 μmol of the mold 60, preferably, for example, 8 to 35 mmol/L, preferably 13 to 22 mmol/L, more preferably 17 to 20 mmol/L. Furthermore, the amount of use of the polymerizable monomer M29 with respect to the polymerizable monomer M28 includes, as the amount of use of the polymerizable monomer M29 with respect to 1 mol of polymerizable monomer M28, 8 to 35 mol, preferably 13 to 22 mol, more preferably 17 to 20 mol.

The polymerization reaction liquid appropriately comprises a crosslinking agent, initiating agent, and solvent.

A crosslinking agent can include a compound wherein two or more ethylenically unsaturated groups are bound by a linker group. An example of a specific crosslinking agent includes the matter shown in Formula (6) below.

[Chemical 15]

W—Z—W (6)

In Formula (6), the W represents ethylenically unsaturated groups that may the same or different with respect to one another, and Z represents a linker group. An ethylenically unsaturated group includes an acrylic group and a methacrylic group. A linker group includes, for example, with the carbon number of 1 to 6, preferably 2 to 6, an alkylene group, amino group (—NH—), ether group, carbonyl group, ester bond (—COO— or —OCO—), amide bond (—CONH— or —NHCO—), sulfoxide group (—SO—), sulphonyl group (—SO₂—), and a group wherein two or more of the above are bound. When two or more of the above-mentioned groups are bound to configure the above-mentioned linker, said bond number is preferably 5 or less or 4 or less, more preferably 3 or less or 2. A more specific example of a crosslinking agent includes a low molecule crosslinking agent such as N,N′-methylene bisacrylamide, and ethylene glycol dimethacrylate, preferably N,N′-methylene bisacrylamide.

The concentration of the crosslinking agent in the polymerization reaction liquid is not particularly limited, but includes, for example, 1 to 10 mmol/L, preferably 3 to 8 mmol/L, more preferably 4.5 to 7 mmol/L, further preferably 5.5 to 6.5 mmol/L.

An initiating agent includes peroxide such as ammonium persulfate and potassium persulfate, and azoic polymerization initiating agent such as azobisisobutyronitrile and 2,2′-azobis(2-methylpropionamidine) dihydrochloride, preferably includes an azoic polymerization initiating agent, more preferably 2,2′-azobis(2-methylpropionamidine) dihydrochloride.

The concentration of the initiating agent in the polymerization reaction liquid is not particularly limited, but includes, for example, 4 to 40 mmol/L, preferably 12 to 32 mmol/L, more preferably 18 to 28 mmol/L, further preferably 22 to 26 mmol/L.

A water-based solvent such as a buffering liquid is used as a solvent, which preferably includes a phosphate buffer liquid. In addition, a dissolving agent preferably comprises NaCl, wherein the concentration of NaCl includes, for example, 100 to 200 mM, preferably 120 to 160 mM. In addition, the pH of the dissolving agent includes, for example, 6 to 8, preferably 6.8 to 7.8, more preferably 7.2 to 7.6.

A specific polymerization method includes a deemulsifying agent precipitation polymerization method, dispersion polymerization method, emulsion polymerization method, seed emulsion polymerization method, and the like, preferably includes deemulsifying agent precipitation polymerization method.

Progression of polymerization reaction in such a polymerization reaction system causes synthesis of the molecularly imprinted polymer 20 and formation of the molecular imprint recessed part 21 complementary to the surface shape of the mold 60. In addition, the molecular imprint recessed part 21 is efficiently formed due to the fact that the mold 60 can easily exist near the polymer boundary surface.

### <3-2-3. Step (1-13)>

In step (1-13), as shown in FIG. 13-3, the first reversible linking group 22 is cleaved to remove the mold 60 from the molecularly imprinted polymer 20 and cause the group 22′ for binding a specific binding molecule. This causes obtainment of the base material 10′ (10-P′) for making a sensor for analysis.

<3-3. Manufacturing Method of a Base Material for Making a Sensor for Analyzing a Detection Subject that is Particle-Like-2>

For example, the manufacturing method of the base material 10c-P′ for making a sensor for analyzing a detection subject that is base plate-type which is shown in FIG. 10(A) comprises the following step (1-21) to step (1-24):
the step (1-21) of preparing a surface of a mold of a molecular imprint wherein the surface is modified with a polymerizable functional group via a third reversible linking group;
the step (1-22) of using a monomer comprising said polymerizable functional group and an interaction group for said mold as a substrate and synthesizing a molecularly imprinted polymer for a part of said surface of said mold;
the step (1-23) of cleaving said third reversible linking group and removing said mold from said molecularly imprinted polymer; and
the step (1-24) introducing a molecule having a functional group comprising a group for binding a specific binding molecule to a target of a detection subject and a group for binding a signal substance via the third reversible linking group.

Provided that said group for binding a specific binding molecule is a group adapted to a small substance.

In addition, when the interaction group 25 for a mold is not comprised as in 10b-PC′ of FIG. 11, only a monomer comprising an interaction group for a mold should be excluded from the substrate in the above-mentioned step (1-22).

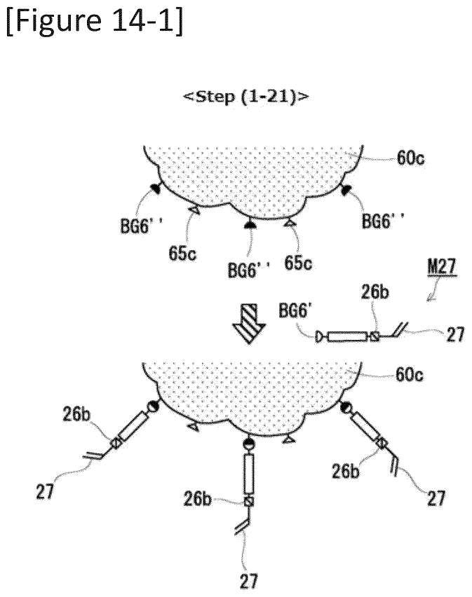

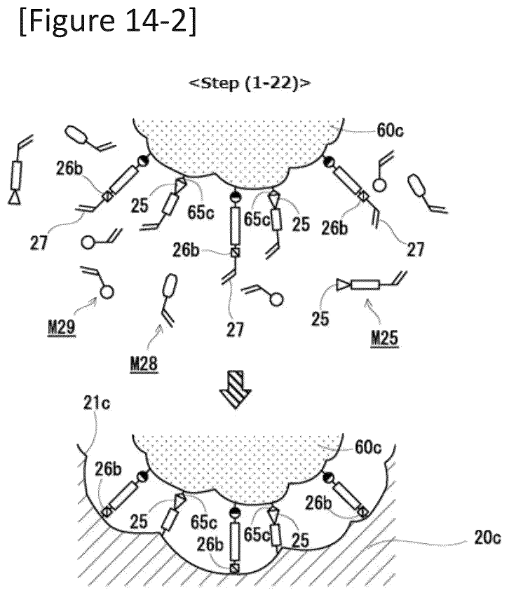

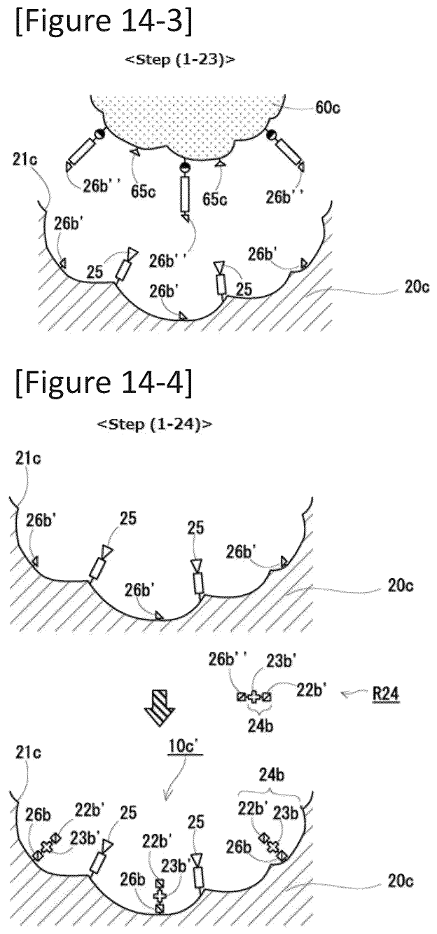

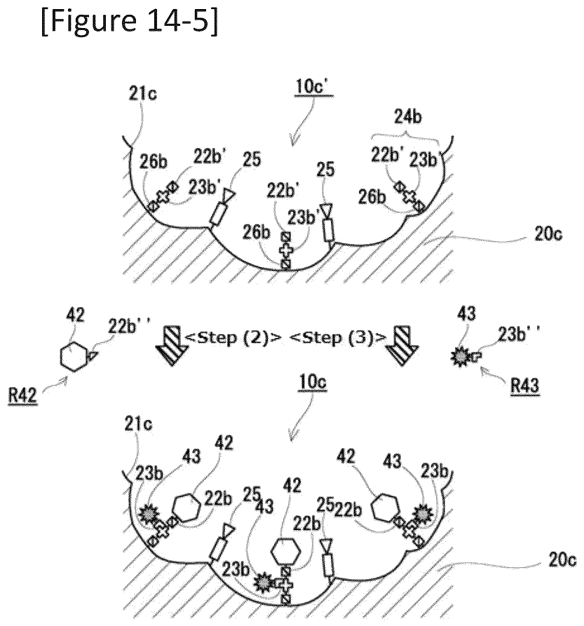

FIG. 14-1 to FIG. 14-4 schematically show a manufacturing method of the base material 10c-P′ for making a sensor for analyzing a detection subject that is base plate-type which is shown in FIG. 10(A). Specifically, FIG. 14-1, FIG. 14-2, FIG. 14-3, and FIG. 14-4 schematically show step (1-21), step (1-22), step (1-13), and step (1-14), respectively. In other words, in the depicted embodiment, the method of manufacturing a base material 10c-P′ for making a sensor for analyzing a detection subject comprises the following steps:
the step (1-21) of preparing the mold 60c of a molecular imprint wherein the surface is modified with a polymerizable functional group 27 via the third reversible linking group 26b;
the step (1-22) of using the monomer M25 comprising the polymerizable functional group 27 and the interaction group 25 for the mold 60c as a substrate and synthesizing the molecularly imprinted polymer 20c for a part of the surface of the mold 60;
the step (1-23) of cleaving the third reversible linking group 26b and removing the mold 60c from the molecularly imprinted polymer 20c; and
the step (1-24) of introducing the functional molecule R24 having the functional group 24b comprising the group 22′ for binding a specific binding group for a target of a detection subject and the group 23′ for binding a signal substance via the third reversible linking group 26b.

Each step is discussed in detail while referring to the drawings.

### <3-3-1. Step (1-21)>

In step (1-21), as shown in FIG. 14-1, the mold 60c of a molecular imprint, wherein the surface is modified with a polymerizable functional group 27 via the third reversible linking group 26b, is prepared.

The mold 60c includes those discussed in “(Mold)” of “3-1-2. Mold and modification step”, but preferably includes a biomolecule, more preferably includes a plasma protein (albumin, γ-globulin, fibrinogen, and the like), more preferably includes albumin.

In the depicted embodiment, the mold 60c itself comprises an interaction site 65c and a binding group BG6″ on the surface. The interaction site 65c is not particularly limited, but includes, for example, the groups shown in column 3-2 of Table 3 above, preferably includes an acidic amino acid residue, more preferably includes a carboxyl group. The binding group BG6″ is not limited, and includes the groups shown in column 2-1 of Table 2 above, preferably includes an amino group.

The monomer M27 comprising the binding group BG6′ corresponding to the binding group BG6″, the third reversible linking group 26b, and the polymerizable functional group 27 is acted on the mold 60c to obtain the mold 60c of a molecular imprint wherein the surface is modified with the polymerizable functional group 27 via the third reversible linking group 26b.

The binding group BG6′ is not particularly limited, but includes, for example, the groups shown in column 2-2 of Table 2 above, preferably includes a carboxylic acid-active ester group. The reversible linking group 26b is not particularly limited, which includes, for example, the groups shown in column 1-3 of Table 1 above, preferably includes a disulfide group. The polymerizable functional group 27 should only contain a vinyl group, which preferably includes a (meth)acryloyl group.

### <3-3-2. Step (1-22)>

In step (1-22), as shown in FIG. 14-2, the polymerizable functional group 27 is used as a substrate to synthesize the molecularly imprinted polymer 20c for a part of the surface of the mold 60.

The depicted embodiment shows an example of using the monomer M25 comprising the interaction group 25 for the mold 60c as well as the polymerization monomers M28 and M29 as a polymerization monomer, but three or more types may be used in combination as a polymerization monomer other than monomer M25. In addition, a polymerizable monomer other than the monomer M25 is as discussed in “1-3-6. A polymer configuring a molecularly imprinted polymer” above. The polymerizable monomer M28 used in the depicted example is a biocompatible monomer, wherein the biocompatible monomer is as discussed in “(A unit derived from a biocompatible monomer)” of “1-3-6. A polymer configuring a molecularly imprinted polymer” above. The polymerizable monomer M29 used in the depicted example is N-substituted or unsubstituted (meth)acrylamide, wherein the N-substituted or unsubstituted (meth)acrylamide is as discussed in the “(A unit derived from N-substituted or unsubstituted (meth)acrylamide)” of “1-3-6. A polymer configuring a molecularly imprinted polymer” above.

In the monomer M25 comprising the interaction group 25 for the mold 60c, the interaction group 25 is as discussed in “1-3-5. Interaction group” above, preferably includes a pyrrolidyl group.

The concentration of the mold 60c in a polymerization reaction liquid; the concentration of the polymerizable monomer M28 and the amount of use of the polymerizable monomer M28 with respect to the mold 60c; and the polymeric monomer M29, the amount of use of the polymeric monomer M29 with respect to the mold 60c and the amount of use of the polymeric monomer M29 with respect to the polymerizable monomer M28 are the same as the concentration of the mold 60 in a polymerization reaction liquid; the concentration of the polymerizable monomer M28 and the amount of use of the polymerizable monomer M28 with respect to the mold 60; and the polymeric monomer M29, the amount of use of the polymeric monomer M29 with respect to the mold 60 and the amount of use of the polymeric monomer M29 with respect to the polymerizable monomer M28 discussed in “3-2-2. Step (1-12)” above.

The concentration of the monomer M25 comprising the interaction group 25 is not particularly limited, but includes, for example, 1.5 to 15 mmol/L, preferably 4.5 to 12 mmol/L, more preferably 6 to 10 mmol/L, further preferably 8.5 to 9.5 mmol/L. In addition, the amount of use of the monomer M25 comprising the interaction group 25 for the mold 60 includes, as the mount of use of the monomer M25 per 1 μmol of the mold 60, for example, 0.15 to 3 mmol, preferably 0.45 to 2.7 mmol, more preferably 0.75 to 2.2 mmol, further preferably 1.2 to 2.2 mmol. In addition, the amount of use of the monomer M25 with respect to the polymerizable monomer M28 includes, as the amount of use of the monomer M25 with respect to 1 mol of the polymerizable monomer M28, for example, 0.3 to 3 mol, preferably 0.5 to 2.5 mol, more preferably 1 to 2 mol, further preferably 1.3 to 1.7 mol. Furthermore, the amount of use of the monomer M25 with respect to the polymerizable monomer M29 includes, as the amount of use of the monomer M25 with respect to 100 mol of the polymerizable monomer M29, for example, 2 to 20 mol, preferably 5 to 13 mol, more preferably 8 to 9 mol.

The polymerization reaction liquid appropriately comprises a crosslinking agent, initiating agent, and solvent. The details of the crosslinking agent, initiating agent, and solvent and the amount of use, polymerization reaction type thereof are as discussed in “3-2-2. Step (1-12)” above.

In the polymerization reaction system of step (1-22), as shown in FIG. 14-2, the monomer M25 comprising the interaction group 25 for the mold 60c among the monomer components specifically interacts with the interaction site 65c of the mold 60c via the interaction group 25 to form a composite of a monomer M25-mold 60c. Progression of polymerization reaction in a state in which such a composite is formed enables formation of the molecular imprint recessed part 21c complementary to the surface shape of the mold 60c as well as presence of the third reversible linking group 26b on the molecular imprint recessed part 21c surface. In addition, the molecular imprint recessed part 21c is even more efficiently formed due to the fact that the mold 60c can even more easily exist near the polymer boundary surface due to hydrophilicity.

In step (1-22), the polymerization reaction liquid may be exposed to a low temperature condition of 1 to 15° C., preferably 1 to 10° C., more preferably 2 to 6° C., in order to sufficiently form a composite of the monomer M25-mold 60c before being exposed to the temperature condition that progresses polymerization reaction. The time of exposure to the low temperature condition includes, for example, 20 to 30 hours. The temperature condition that progresses polymerization reaction includes, for example, 40 to 76° C., preferably 50 to 74° C., more preferably 60 to 72° C. The time of exposure to the temperature condition that progresses polymerization reaction includes, for example, 10 to 20 hours. Furthermore, even when polymerization reaction is carried out under the heating condition, the shape of the mold 60c is retained to the extent of not causing a denaturation that causes loss of the specificity of a molecular imprint space, and thus it is possible to effectively acquire specificity of the molecular imprint recessed part 21c.

As such, the molecularly imprinted polymer 20c is synthesized, and a composite of the molecularly imprinted polymer 20c and the mold 60c is obtained. The composite of the molecularly imprinted polymer 20c and the mold 60c is formed by a non-covalent bond between the interaction group 25 and the interaction site 65c of the mold 60c.

### <3-3-3. Step (1-23)>

In step (1-23), as shown in FIG. 14-3, the third reversible linking group 26b is cleaved to remove the mold 60c from the molecularly imprinted polymer 20c. This causes the interaction group 25 and the binding group 26b′ to be provided on the recessed part 21c surface of the molecularly imprinted polymer 20c. The binding group 26b′ is a group that occurs by the cleavage of the third reversible linking group 26b, which specifically includes the groups shown in column 1-1 of Table 1 above, preferably includes a thiol group.

### <3-3-4. Step (1-24)>

In step (1-24), as shown in FIG. 14-4, the functional molecule R24 having the functional group 24b comprising the group 22′ for binding a specific binding molecule to a target of a detection subject and the group 23′ for binding a signal substance is introduced the recessed part 21c of the polymer 20c via the third reversible linking group 26b.

The functional group 24b comprising the group 22′ for binding a specific binding molecule to a target of a detection subject and the group 23′ for binding a signal substance is as discussed in “1-3-4. Functional group” above. The functional molecule R24 having the functional group 24b is a molecule having the binding group 26b″ for the binding group 26b′, which is represented by Formula (7) below.

[Chemical 16]

R⁷-L⁷-R²-L¹-R¹ (7)

In Formula (7), R¹ represents a group for binding a specific binding molecule, L² represents a direct binding or linking group, and R² represents a linking group comprising a group for binding a signal substance, wherein the details of these groups are as discussed in “1-3-4. Functional group” above.

In Formula (7), R⁷ is the binding group 26b″, and L⁷ represents a direct binding or linking group. The binding group 26b″ is not particularly limited as long as the group has binding property for the binding group 26b′, which includes, for example, the groups shown in column 1-2 of Table 1 above. When the direct binding or linking group L⁷ is a linking group, a specific example of said linking group includes an alkylene group that may comprise a polyoxyalkylene group (e.g., C1 to 8 alkylene group, or polyoxy C1 to 3 alkylene C1 to 8 alkylene group), carbonyl group, ester bond (—COO— or —OCO—), amide bond (—CONH— or —NHCO—), and a group consisting of any combination thereof. From the viewpoint of molecular design and the like, said linking group includes preferably a combination of an alkylene group and an ester bond and/or amide bond, more preferably a combination of an alkylene group and an ester bond or an amide bond, further preferably a combination of one or two or more alkylene group (the total of the carbon number of all alkylene groups comprised in said linking group is, for example, 1 to 8, preferably 2 to 6, more preferably 3 to 5, especially preferably 4) and an ester bond or an amide bond.

The specific unlimited example of the functional molecule R24 having the functional group 24b includes the compound shown in Formulas (7a), (7b), and (7c) below.

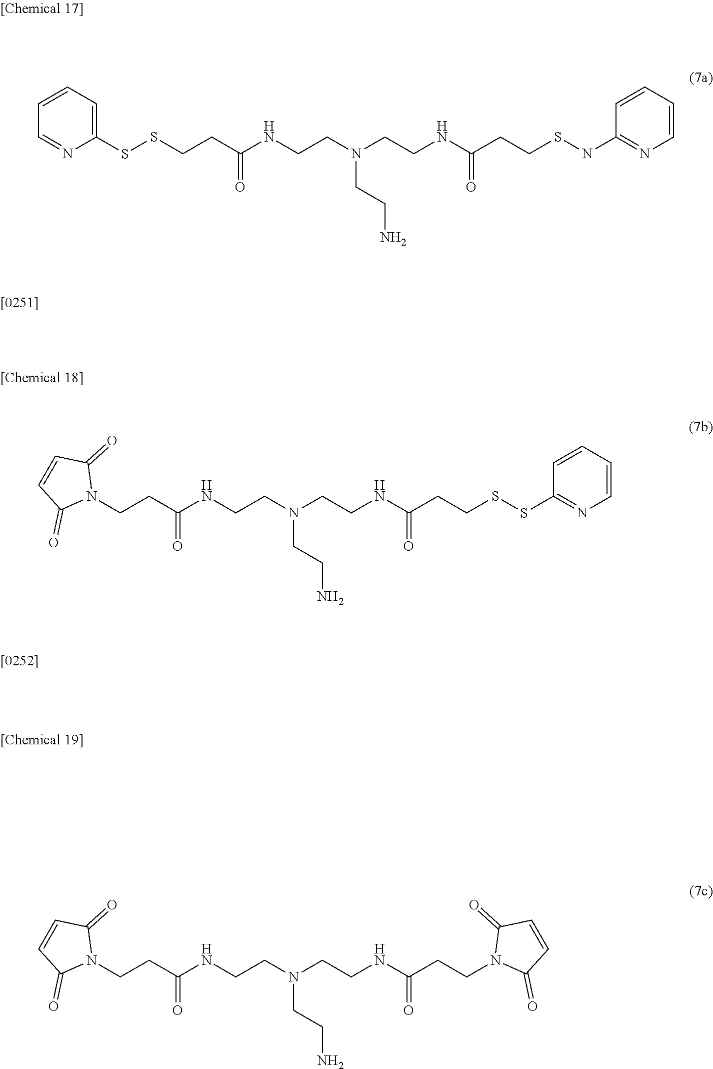 [SMILES: NCCN(CCNC(=O)CCSNc1ccccn1)CCNC(=O)CCSSc1ccccn1 ; NCCN(CCNC(=O)CCSSc1ccccn1)CCNC(=O)CCN1C(=O)C=CC1=O ; NCCN(CCNC(=O)CCN1C(=O)C=CC1=O)CCNC(=O)CCN1C(=O)C=CC1=O]

As such, the functional molecule R24 having the functional group 24b is introduced to the recessed part 21c of the polymer 20c via a third reversible linking group 26b, whereby the base material 10c′ (10c-P′) for making a sensor for analysis is obtained.

### <3-4. Manufacturing Method of a Base Material for Making a Sensor for Analyzing a Detection Subject Wherein a Particle is Immobilized on a Base Plate>

As shown in FIG. 7(A) and FIG. 11(A), the manufacturing method of the base material for making a sensor for analyzing a detection subject wherein a particle is immobilized on a base plate should only be an appropriate combination of the manufacturing method discussed in “3-2. Manufacturing method of a base material for making a sensor for analyzing a detection subject that is particle-like-1” and “3-3. Manufacturing method of a base material for making a sensor for analyzing a detection subject that is particle-like-2” above and the method of fixing a polymer particle on the base plate 30.

The step of fixing a polymer particle on the base plate 30 can be carried out any time after forming a particle of a molecularly imprinted polymer (e.g., the molecularly imprinted polymers 10 and 10c) where a mold (e.g., the molds 60 and 60c) is removed in the manufacturing method discussed in “3-2. Manufacturing method of a base material for making a sensor for analyzing a detection subject that is particle-like-1” and “3-3. Manufacturing method of a base material for making a sensor for analyzing a detection subject is particle-like-2” above. For example, it is possible to fix the particle of the obtained molecularly imprinted polymers 10 and 10c on the base plate 30 after the “3-2-3. Step (1-13)” above, after the “3-3-3. Step (1-23)” above (preferably between the “3-3-3. Step (1-23)” above and the “3-3-4. Step (1-24)” above), or the like. The method of fixing a particle of a molecularly imprinted polymer on the base plate 30 is not particularly limited, wherein those skilled in the art can appropriately determine a common method of fixing a polymer nanoparticle to a base plate surface.

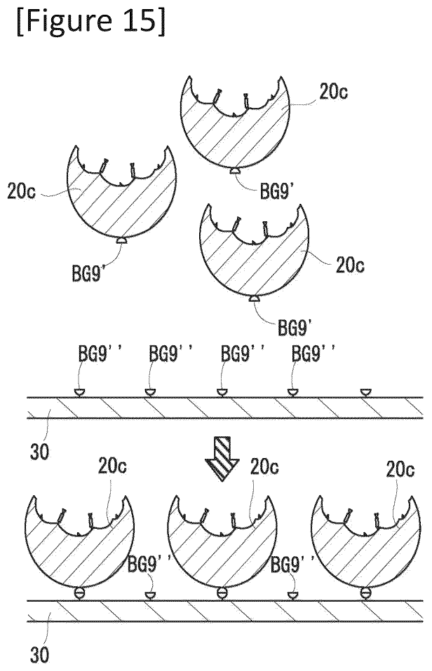

FIG. 15 schematically shows the step of fixing a particle of the molecularly imprinted polymer 20c on the base plate 30 between the “3-3-3. Step (1-23)” above and the “3-3-4. Step (1-24)” above. In the example shown in FIG. 15, a molecular film having the binding group BG9″ on the surface is formed on the base plate 30 surface and the binding group BG9′ existing on the surface of the molecularly imprinted polymer 20c is bound to the binding group BG9″ to fix the molecularly imprinted polymer 20c on the base plate 30.

The functional group BG9″ of the molecular film surface formed at the base plate 30 is not particularly limited, but includes, for example, the groups shown in column 2-2 of Table 2 above, preferably including a carboxylic acid-active ester group. The binding group BG9′ that exists on the surface of the molecularly imprinted polymer 20c is not particularly limited, which includes, for example, the groups shown in column 2-1 of Table 2 above. The binding group BG9′ that exists on the surface of the molecularly imprinted polymer 20c includes a group derived from a monomer used for synthesis of the molecularly imprinted polymer 20c and/or a group derived from an initiating agent that remains at the terminal of a polymer configuring the molecularly imprinted polymer 20c, and the like.

### <4. Manufacturing Method of a Sensor for Analyzing a Detection Subject>

The manufacturing method of a sensor for analyzing a detection subject of the present disclosure comprises the following steps:
the step (1) of carrying out the manufacturing method of a base material for making a sensor for analyzing a detection subject; and
the step (2) of binding said specific binding molecule to said group for binding a specific binding molecule.

FIG. 13-4 schematically shows the manufacturing method of the sensor 10 (10-P) for analyzing a detection subject of the present disclosure. In other words, in the embodiment shown in FIG. 13-4, the manufacturing method of the sensor 10 (10-P) for analyzing a detection subject comprises the following steps:
the step (1) of carrying out the manufacturing method of the base material 10′ (10-P′) for making a sensor for analyzing a detection subject; and
the step (2) of binding a specific binding molecule R42 to the group 22′ for binding a specific binding molecule.

In addition, the manufacturing method of a sensor for analyzing a detection subject of the present disclosure of the case of comprising a group for binding signal substance in the base material for making a sensor for analyzing a detection subject comprises the following steps:
the step (1) of carrying out the manufacturing method of a base material for making a sensor for analyzing a detection subject;
the step (2) of binding said specific binding molecule to said group for binding a specific binding molecule; and
the step (3) of binding a signal substance to said group for binding a signal substance.

FIG. 12-6 and FIG. 14-5 schematically show the manufacturing method of the sensors 10a-C and 10c (10c-P) for analyzing a detection subject of the present disclosure. In other words, in the embodiment shown in FIG. 12-6 and FIG. 14-5, the manufacturing method of the sensors 10a-C and 10c (10c-P) for analyzing a detection subject comprises the following steps:
the step (1) of carrying out the manufacturing method of the base materials 10a-C′ and 10c′ (10c-P′) for making a sensor for analyzing a detection subject;
the step (2) of binding the specific binding molecule R42 to a group for binding the specific binding molecule 22′; and
the step (3) of binding the signal substance R43 to the group 23′ for binding a signal substance.

Step (1) is as discussed in the “3. Manufacturing method of a base material for making a sensor for analyzing a detection subject” above. When the manufacturing method of a sensor for analyzing a detection subject of the present disclosure carries out step (3), as for the order of step (2) and step (3), either can be carried out first, or they can be carried out simultaneously.

The specific binding molecule R42 comprises the specific binding group 42 for target of a detection subject and binding groups 22″ and 22bh″. The specific binding group 42 for a target of a detection subject is as discussed in the “2-2. A specific binding group for a target of a detection subject” above. Furthermore, although not depicted, the manufacturing method of the sensor 10a₁-C for analysis (FIG. 8(B2)) should only carry out the same procedure as FIG. 12-6 excluding the use of a molecule comprising the specific binding group 42₁ (see FIG. 2B-2) having a different signal, wherein a different signal sg is bound to the specific binding group 42, and the binding group 22″ as the specific binding molecule R42.

In addition, the binding group 22″ is not particularly limited as long as the group has binding property to the group 22′ for binding a specific binding molecule, which includes, for example, the groups shown in column 1-2 of Table 1 above. The binding group 22b″ is not particularly limited as long as the group has binding property to the group 22′ for binding a specific binding molecule, which includes, for example, the groups shown in column 2-2 of Table 2 above.

A signal substance R43 comprises the signal group 43 and the binding groups 23″ and 23b″. The signal group 43 is as discussed in “2-3. Signal group” above. In addition, the binding group 23″ is not particularly limited as long as the group has binding property to the group 23′ for binding a signal substance, which includes, for example, the groups shown in column 1-2 of Table 1 above. The binding group 23b″ is not particularly limited as long as the group as binding property to the group 23′ for binding a signal substance, which includes, for example, the group of column 2-2 of Table 2 above.

In a manufacturing method of a sensor for analyzing a detection subject of the present disclosure (e.g., the sensor 10a-C for analyzing a detection subject discussed above, or the like) which is base plate-type and has the signal group 43, a group for binding a signal substance is only provided at a molecular imprint recessed part that forms a sensor field on the base plate 30, which enables the signal group 42 to be disposed only at a molecular imprint recessed part due to the reaction property of the binding group of the signal substance R42.

In addition, regarding the sensor for analyzing a detection subject of the present disclosure that is base plate-type and has the signal group 43, it is possible to introduce one type of specific binding group 42 and one type of signal group 43 to all molecular imprint recessed parts on one base plate 30, and it is also possible to introduce one type of specific binding group 42 to one molecular imprint recessed part, introduce another type of specific binding group 42 to another molecular imprint recessed part, and introduce different types of signal groups 43 corresponding to the types of the specific binding group 42, respectively.

### <5. Analysis Method of a Subject Substance>

The analysis method of a detection subject of the present disclosure comprises: the step of contacting a sample comprising a detection subject to a sensor for analyzing a detection subject and binding said detection subject to said specific binding group; and the step of detecting the bond between said specific binding group and said detection subject.

### <5-1. Sensor for Analyzing a Detection Subject>

The sensor for analyzing a detection subject is as discussed in “2. Sensor for analyzing a detection subject” above.

### <5-2. Detection Subject>

The detection subject is not particularly limited as long as the target of the detection subject is a subject that specifically binds to the specific binding group 42, which includes those discussed in “2-1. Detection subject” above.

The form of the analysis sample liquid comprising a detection subject is not particularly limited, but from the viewpoint of rapidity of analysis, those that did not go through the treatment of separating the detection subject is included. The treatment of separating a detection subject includes ultracentrifugation, ultrafiltration, successive flow electrophoresis, filtration using a size filter, gel filtration chromatography, and the like.

The analysis sample liquid comprising a detection subject may be a sample obtained from an environment where a detection subject exists (when the detection subject is a cell or an extracellular vesicle), or a sample obtained from an environment where a detection subject may be generated (when the detection subject is an extracellular vesicle and is a product or discharge from a cell). Specifically, the analysis sample liquid may be a biological sample comprising a cell. When the detection subject is an extracellular vesicle such as exosome, a cell that produces or discharges the detection subject includes a cancer cell, mast cell, dendritic cell, reticulocyte, epithelial cell, B cell, nerve cell, mucosal cell, and the like. More specifically, an analysis test liquid comprising a detection subject includes body fluids such as blood, milk, urine, saliva, lymph, cerebrospinal fluid, amniotic fluid, tear fluid, sweat, nasal discharge, throat swab, nasopharyngeal liquid, and the like, and further includes a treatment liquid wherein these body fluids underwent pretreatment such as removal of unnecessary components, and culture liquid obtained by culturing cells comprised in these body fluids. Among these analysis test liquids, body fluids such as urine, saliva, tear fluid, sweat, and nasal discharge are particularly preferable in terms of non-invasiveness and easiness of collection.

When a sample comprising a detection subject is contacted to a sensor for analyzing a detection subject, the detection subject or a target of the detection subject is specifically captured by the specific binding group 42 within a molecular print recessed part.

### <5-3. Targeting and Sensing>

When a detection subject or a target of the detection subject is specifically captured by the specific binding group 42 within a molecular print recessed part, the bond between the specific binding group and said detection subject is detected.

For example, when a sensor for analyzing a detection subject does not have the signal group 43 within a molecular print recessed part like the sensor 10 for analyzing a detection subject shown in FIG. 1B-1, said bond can be detected by surface plasmon resonance (SPR), reflectometric interference spectroscopy, or the like.

In addition, when a sensor for analyzing a detection subject has the signal group 43 within a molecular print recessed part, since the signal group 43 would be affected by the environmental change by the detection subject or a target of the detection subject at the moment of binding, before and after the specific capturing, a signal would change. In other words, in this case, the sensor for analysis can read binding information of a detection subject or a target of the detection subject that would be a sensing subject with a signal change, which signal change causes detection of the detection subject or the target of the detection subject. Since the capturing of the detection subject or the target of the detection subject and the signal change occur almost simultaneously, there is no need to add a reagent for detection of binding, and detection can be promptly carried out.

In addition, in the case wherein a sensor for analysis is configured so that the signal group 43 would be one of the fluorescent dye pair that causes fluorescence resonance energy transfer (FRET), and one of said fluorescent dye pair is bound to a detection subject or a target of the detection subject beforehand, when the detection subject or the target of the detection subject is specifically captured by the specific binding group 42 within a molecular imprint recessed part, at the moment thereof, the fluorescent dye in the signal group 43 and the fluorescent dye in the detection subject would get close, and thus fluorescence is radiated by FRET. The detection subject is detected by this fluorescence radiation by this FRET. Since the capturing of a detection subject or a target of the detection subject and fluorescence radiation by FRET occur almost simultaneously, there is no need to add a reagent for detection of the detection subject, and detection can be promptly carried out. The fluorescent dye pair that causes FRET is not particularly limited, and it is not limited which of a donor dye and acceptor dye is selected as the signal group 43. Preferably, it is possible to select a donor dye as the signal group 43. A specific example of the donor dye/acceptor dye configuring a pair of fluorescent dyes that causes FRET includes fluorescein isothiocyanate (FITC)/tetramethylrhodamine isothiocyanate (TRITC), Alexa Fluor647/Cy5.5, HiLyte Fluor647/Cy5.5, R-phycoerythrin (R-PE)/allophycocyanin (APC), and the like.

### <5-4. Double Targeting and Sensing>

In addition, FIG. 16-1 and FIG. 16-2 schematically show the targeting trend of the case wherein the sensor 10c-P for analyzing a detection subject shown in FIG. 10(B) has a molecular imprint recessed part 21c which uses albumin (may be other plasma protein) as a mold 60c and a specific interaction group 25 for albumin and is used for the purpose shown in “2-4. Usage” above. The depicted example shows the case wherein the detection subject 70 of the sensor 10c-P for analysis is a microparticle having a film structure like exosome and the target 72 which is a direct target is a tumor marker protein expressed on the surface of said microparticle (specific example of the case of being “Embodiment 2” of “2-1. Detection subject” above). In addition, FIG. 16-1 and FIG. 16-2 both schematically show both the enlarged view (i) of the molecular imprint recessed part 21c and the total view (ii) of the sensor 10c-P for analysis of a detection subject corresponding to (i).

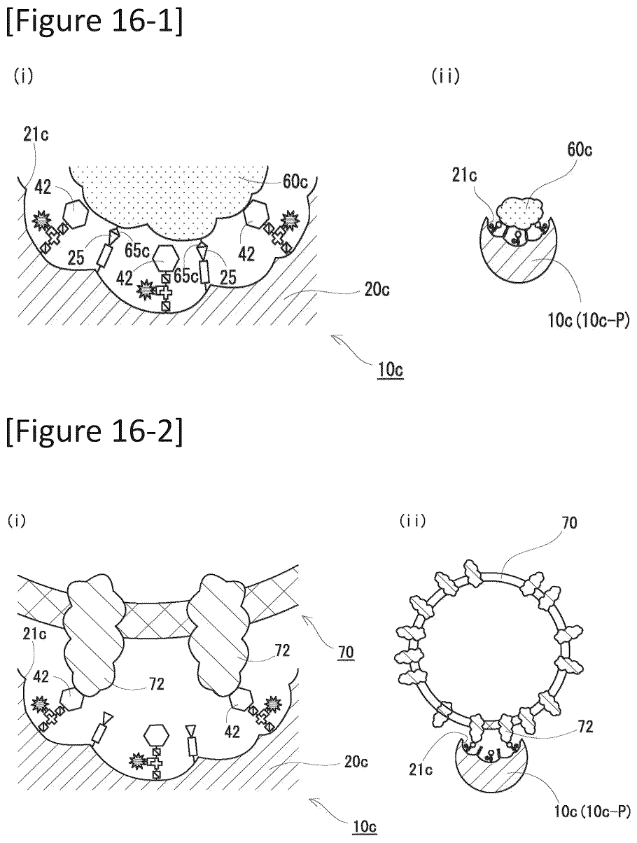

As shown in FIG. 16-1, when the sensor 10c-P for analysis is administered in blood, first, albumin which is the many plasma proteins that exist in blood (same substance as the mold 60c) is specifically captured by the molecular imprint recessed part 21c which uses the albumin in the sensor 10c-P for analysis as a mold and a specific interaction group 25 for albumin. This causes the sensor 10c-P for analysis to capture the stealth property in vivo. In this case, the specific binding group 42 is shielded by the captured albumin (same substance as the mold 60c).

Meanwhile, when the sensor 10c-P for analysis that captured the stealth property reaches a tumor tissue, the albumin concentration would decrease in the tumor tissue, and thus, as shown in FIG. 16-2, the captured albumin would be taken out, exposing the specific binding group 42, and a tumor marker protein (target 72) specifically binds to the exposed specific binding group 42. This binding information is detected as a signal change based on the signal group. Furthermore, when the detection subject 70 is a microparticle having a film structure and the target 72 thereof is set as a direct capturing subject as shown in FIG. 16-2 (ii), the molecular imprint recessed part 21c does not have to be in the size that can receive the entirety of the detection subject 70, but should only be in the size that can receive the target 72 near the specific binding site.

As such, in in the embodiment shown in FIG. 16-1 and FIG. 16-2, the sensor 10c-P for analysis enables double targeting by the difference in the environment (albumin concentration) where the sensor is exposed. When the object is such double targeting, the combination of the mold 60c of the molecular imprint recessed part 21c and the interaction group 25 and the specific binding group 42 is not limited to the embodiment shown in FIG. 16-1 and FIG. 16-2, and can be optionally selected.

In the discussion above, explanation has been provided while referring to the case wherein the sensor 10c-P for analyzing a detection subject has the molecular imprint recessed part 21c which uses albumin as the mold 60c and the specific interaction group 25 for albumin and is administered in blood to be delivered near a tumor, but the double targeting is not limited to this example. As long as the sensor 10c-P for analyzing a detection subject has the molecular imprint recessed part 21c and the interaction group 25 for the mold thereof, regardless of what kind of mold is used as the mold 60c, the embodiment shown in FIG. 16-1 would be achieved in an environment wherein the concentration of the mold 60c of the molecular imprint recessed part 21c is high, and the embodiment shown in FIG. 16-2 would be achieved in an environment wherein the concentration of the mold 60c of the molecular imprint recessed part 21c is low.

In one embodiment, a method of diagnosing and examining a subject using the sensor for analysis of the present disclosure, comprises:
A) the step of collecting a sample (comprising, for example, urine, cerebrospinal fluid, sweat, nasal mucus, saliva, blood, or tear) of a subject;
B) the step of contacting the sample with the sensor for analysis;
C) the step of detecting a signal from the sensor for analysis; and
D) the step of analyzing the signal and then diagnosing the subject (e.g., determining presence/absence or progression of a disease).

In one embodiment, a program that causes a computer to execute a method of diagnosing and examining a subject using a diagnosis or examination system comprising the sensor for analysis of the present disclosure, comprises:
A) the step of providing a sample (comprising, for example, urine, cerebrospinal fluid, sweat, nasal mucus, saliva, blood, or tear) obtained from the subject to the diagnosis or examination system;
B) the step of activating the diagnosis or examination system to contact the sample with the sensor for analysis;
C) the step of the diagnosis or examination system analyzing a signal from the sensor for analysis; and
D) the step of the diagnosis or examination system analyzing the signal and then diagnosing or detecting the subject (e.g., determining presence/absence or progression of a disease from a detection result).

In one embodiment, a record medium storing a program that causes a computer to execute a method of diagnosing or examining a subject using the sensor for analysis of the present disclosure, comprises:
A) the step of providing a sample (comprising, for example, urine, cerebrospinal fluid, sweat, nasal mucus, saliva, blood, or tear) obtained from the subject to the diagnosis or examination system;
B) the step of activating the diagnosis or examination system to contact the sample with the sensor for analysis;
C) the step of the diagnosis or examination system analyzing a signal from the sensor for analysis; and
D) the step of the diagnosis or examination system analyzing the signal and then diagnosing or detecting the subject (e.g., determining presence/absence or progression of a disease from a detection result).

In one embodiment, a diagnosis or examination system, comprises:
A) a providing unit that provides a sample (comprising, for example, urine, cerebrospinal fluid, sweat, nasal mucus, saliva, blood, or tear) obtained from the subject;
B) an activation unit that activates so as to contact the sample to the sensor for analysis;
C) a detection unit that detects a signal from the sensor for analysis; and
D) a diagnosis unit that analyzes the signal and then provides diagnosis to or examines the subject (e.g., determines presence/absence or progression of disease from a detection result).

Said step B) to step D) can all be fully automatically carried out, wherein when they are fully automatically carried out, the steps can be carried out using machine learning and/or artificial intelligence, and the method, program, record medium which is a record thereof, and system which realize these steps are also within the scope of the present disclosure.

Generally, the various exemplified embodiments of the present disclosure may be implemented with a hardware or dedicated circuit, software, logic, or any combination thereof. Some embodiments may be implemented with a hardware, and other embodiments may be implemented with a firmware or software that can be executed by a controller, microprocessor, or other computing devices. While the exemplary embodiment of the present disclosure can be shown as a block diagram, flowchart, or using other several graphical expressions, it is understood that the block, apparatus, system, technique, or method described herein may be implemented with, as a non-limiting example, hardware, software, firmware, dedicated circuit or logic, general hardware or controller, or other computing devices, or some sort of combination thereof.

In one embodiment, the present disclosure also provides at least one computer program product tangibly stored on a non-transitory computer readable medium, which realizes the technique of the present disclosure. The computer program product comprises a computer executable order such as those comprised in a program module, which is executed by a device on an actual processor or on a virtual processor that is subjected in order to execute the method/process described in herein. Generally, a program module comprises a routine, program, library, object, class, component, data structure, and the like, which execute a specific task or implement a specific abstract data type. The function of the program module may be combined or divided between program modules as desired in various embodiments. The computer executable order for a program module may be executed within a local device or a dispersion device. In the dispersion device, the program module may be disposed in both local and remote storage media.

The storage medium of the present disclosure may be a computer readable medium, and the computer readable medium may be a computer readable signal medium or computer readable storage medium. The computer readable medium includes, but not limited to, an electronic, magnetic, optical, electromagnetic, infrared, or semiconductor system, apparatus, or device, or any appropriate combination of the above. Amore specific example of a computer readable storage medium includes one or more wired electrical connection, portable computer diskette, hard disk, random access memory (RAM), read-only memory (ROM), erasable programmable read-only memory (EPROM or flash memory), optical fiber, portable compact disk read-only memory (CD-ROM), optical storage device, magnetic storage device, or any appropriate combination of the above.

The computer program code for executing the method disclosed herein may be written in any combination of one or more programming language. When a program code is executed by a processor or a controller, the program code may be provided to a general computer dedicated computer, or processor or controller of other programmable data treatment apparatuses so as to execute the function/operation designated in the flowchart and/or block diagram. A program code may be executed completely on a computer, executed partially on a computer as a stand-alone software package, executed partially on a computer and partially on a remote computer, or executed completely on a remote computer or a server. A program code may be distributed to a specially programmed device that may be generally called a “module” herein. The software component portion of the module may be written in any computer language, may be a portion of a monolithic code base, or may be developed at a more discrete code portion as it is typical in an object-oriented computer language. In addition, the module may be dispersed throughout a plurality of computer platforms, servers, terminals, mobile devices, or the like. Furthermore, a predetermined module may be implemented so that an explained function would be executed by a separate processor and/or computing hardware platform.

While the operation is shown in a specific order, in order to obtain a desired result, it should not be understood that such operation should be executed in the shown specific order or sequence, or all of the depicted operations are requested to be executed. In certain situations, multitask and parallel treatment may be advantageous. In the same manner, several specific implementation details are comprised in the explanation above, but they should not be interpreted as restriction to the scope of the present disclosure. Rather, they should be interpreted as explanations of a feature that may be unique to the specific embodiment. Certain features explained in the context of a different embodiment may be combined and implemented in a single embodiment. On the other hand, various features explained in the context of a single embodiment may be implemented separately or in any partial combination in a plurality of embodiments.

In the case of using the technique of the present disclosure, the technique is not intended to be used only to a human, but can also be used for other animals other than a human (cat, dog, cow, horse, bat, fox, mongoose, raccoon, and the like).

(Application Example)
1. Examination of a plague such as cancer of animals that are pets (cat, dog, bird, and the like)
2. Detection of infection-causing virus, infectious protein (prion, and the like), and other pathogenic/infectious factors held by domestic animals (cow, horse, pig, chicken, and the like)
3. Detection of infection-causing virus, infectious protein (prion, and the like), and other pathogenic/infectious factors held by harmful animal (bird, bat, fox, mongoose, raccoon, and the like)
4. Quality and freshness check of aquatic products
5. Quality and freshness check of farm products
6. Pathogenic bacteria/virus check of plants, trees, and the like

The present disclosure has been explained above while showing a preferable embodiment for easy understanding. The present disclosure is explained below based on the Examples, but the explanation above and the Examples below are provided only for the purpose of exemplification, and are not provided for the purpose of limiting the present disclosure. Therefore, the scope of the present disclosure is not limited by the embodiments or the Examples specifically described herein, but is limited only by the Claims.

### EXAMPLES

The present disclosure is explained in detail below based on the Examples, but the present disclosure is not limited thereto. The used reagent can be obtained as shown by various descriptions, other reagents of Merck, Sigma-Aldrich, Funakoshi, and CosmoBio may be used.

CKX31 (OLYMPUS, Tokyo, Japan) was used for the microscopy of cells, KUBOTA2800 (KUBOTA, Tokyo, Japan) was used as a centrifugal separator, CO2 WATER JACKETED INCUBATOR (Thermo Fisher Scientific Inc, Massachusetts, USA) was used as an incubator, KS-243 (TOMY SEIKO Co, Ltd., Tokyo, Japan) was used as an autoclave, and a biological clean bench (ORIENTAL GIKEN INC, Tokyo, Japan) was used as a clean bench. qNano Gold (Izon Science Ltd., Christchurch, New Zealand) was used for exosome concentration measurement. In addition, UV Ozone Cleaner (BioForce Nanosciences, Inc.) was used for the UV ozone treatment of a gold base plate, fluorescence microscope (Olympus Corporation, Tokyo, Japan) with a SIC automatic dispensing apparatus (SYSTEM INSTRUMENTS Co., Ltd., Tokyo, Japan) was used for fluorescence measurement, and Andor SOLIS (Andor Technology Ltd, Belfast, Northern Ireland) was used as a spectroscopic software.

In addition, MALDI-TOF/MS (MALDI-7090, SHIMADZU CORPORATION) was used as MALDI-TOF-MS, MALDI-TOF-MS analysis software (MALDI Solutions, SHIMADZU CORPORATION) was used, protein calibration standard I (BRUKER) was used for carburation. Sinapic acid was used for a matrix. J-725 Circular Dichroism spectrometer (JASCO Corporation, Tokyo, Japan) was used for CD spectrum. The pH was measured by a table pH meter F-52 (HORIBA, Kyoto, JAPAN) for buffer preparation. Fluorescence spectrum measurement was carried out using the Fluorescence spectrophotometer F-2500 (Tokyo, Japan) made by Hitachi High-Tech. Amicon Ultra-4 (10 kDa) was used for the ultrafiltration film used for ultrafiltration. Thermo Scientific™ Nano drop™ One ultramicro-ultraviolet-visible spectrophotometer (Thermo Fisher) was used for absorption spectrum measurement. The average particle size and polydispersion index (PDI) of the particle that was made were measured by Dynamic Light Scattering (DLS) with ZETASIZER NANO-ZS MAL500735 (Malvern, UK), and Zetasizer software was used as an analysis software. A transmission electron microscope (JEM-1230, made by JEOL) was used for TEM analysis.

### Example 1: Analysis Sensor 10a₁-C of a Detection Subject that is Base Plate-Type (Introducing an Anti-HER2 Affibody as a Specific Binding Substance and Alexa Fluor 647 as a Signal Substance

In the present Example, an analysis sensor 10a₁-C of a detection subject that is base plate-type (introducing AlexaFluor 647 as a signal substance) was made. This analysis sensor of a detection subject that is base plate-type set exosome secreted by an SK-BR-3 cell which is an HER2 overexpress-type breast cancer cell line as the detection subject, set HER2 as the target of the detection subject, and selected and introduced anti-HER2 affibody as a specific binding molecule. In addition, Alexa Fluor 647 was introduced as a signal substance. Furthermore, the detection subject was analyzed using the obtained analysis sensor of a detection subject that is base plate-type.

Regarding the method of making an analysis sensor of a detection subject that is base plate-type, a base plate 10a-C′ for making an analysis sensor of a detection subject that is base plate-type was made in accordance with FIG. 12-1, FIG. 12-2, FIG. 12-3a, FIG. 12-4, and FIG. 12-5, and an analysis sensor 10a₁-C of the detection subject was made in accordance with FIG. 12-6 (provided that a molecule comprising a specific binding group 42₁ (see FIG. 2B-2) having a different signal group and binding group 22″, wherein a different signal group sg is bound to the specific binding group 42, was used as the specific binding molecule R42).

<1> Making of a Silica Particle Modified with a Polyhistidine Tag and a Thiol Group

In accordance with FIG. 12-1a, a mold silica particle modified with a polyhistidine tag and a thiol group was made.

### <1-1> Reagent

Sicastar®-red F (micromod; average particle size of 200 nm; solid component concentration of 25 mg/mL; the functional group of the particle surface is a carboxyl group (1 μmol/g); the fluorescence property is 569 nm excitation, 585 nm fluorescence)
   Mold 60: silica particle
   Binding group BG22″: carboxyl group
2-aminoethanethiol hydrochloride (Tokyo Chemical Industry)
   Binding group BG12′: amino group
   Binding group 23″: thiol group
polyhistidine tag (Thermo Fisher Scientific; His-tag; Ac-KHHHHHH-NH₂; Ac represents acetyl group, K represents lysine residue, and H represents histidine residue)
   Binding group BG22′: amino group
   Binding group 22″: polyhistidine
DMT-MM (4-(4,6-dimethoxy-1,3,5-triazine-2-yl)-4-methylmorpholiniumchloride; triazine-based condensing agent)

### <1-2> Experimental Procedure

A silica particle modified with a polyhistidine tag and a thiol group was made by adding 20 equivalent of 2-aminoethanethiol hydrochloride, polyhistidine tag and DMT-MM to the carboxyl group of the Sicastar®-red F surface and shaking (25° C., overnight). Then, a dispersion medium was substituted with water by centrifugal separation (10,000 g, 15 minutes). Furthermore, the operation of substituting a dispersion medium with water by centrifugal separation (10,000 g, 10 minutes) was carried out two times. 100 μL of the made silica particle was collected, and the dry weight was measured. The solid component concentration of the obtained silica particle was 7.41 mg/mL.

### <2> Formation of a Monolayer (Step (1-1))

In accordance with FIG. 12-2, a monolayer having a NTA-Ni complex and a polymerization initiating group on the surface was formed at the base plate.

### <2-1> Reagent

Amino-EG₆-undecanethiol hydrochloride (DOJINDO LABORATORIES)
   Binding group BG1′: amino group
2-(2-bromoisobutyryloxy) undecyl thiol (SURF MODS)
   Polymerizable initiating group INI: —CBr group
Isothiocyanobenzyl-NTA (DOJINDO LABORATORIES), anhydrous NiCl₂ (NACALAI TESQUE)
   Group 22′ for binding a specific binding molecule: nickel-nitrilotriacetic acid-driven group

### <2-2> Experimental Procedure

After a gold sputtered or coated glass base plate was washed with ethanol, UV ozone treatment (20 minutes) was carried out to remove the organic matter on the gold base plate surface. Then, the base plate was immersed in an ethanol solution comprising 0.5 mM of amino-EG6-undecanthiol hydrochloride and 0.5 mM of 2-(2-bromoisobutyryloxy) undecyl thiol and settled (25° C., overnight) to form a mixed self-assembled monolayer (mixed SAMs).

After washing the base plate with pure water, 35 μL of the base plate was dropped in a 5 mM of an isothiocyanobenzyl-NTA solution (prepared by dissolving in 50 μL of DMSO to be prepared to 46 mM, and then diluting with a carbonate buffer of 10 mM, pH9.2) and settled (room temperature, 2 hours). After washing the base plate with pure water, the base plate was immersed in 4 mM of NiCl₂ aqueous solution and settled (room temperature, 15 minutes) to form a NTA-Ni complex (nickel-nitrilotriacetic acid-driven group) on the mixed SAM of the base plate surface, and then the base plate was washed with pure water.

### <3> Introduction of a Mold (Step (1-2))

In accordance with FIG. 12-3a, a mold was introduced to the base plate, and the surface of the mold was modified with a methacryloyl group.

### <3-1> Reagent or the Like

M23₁: 2-(2-pyridyl)dithioethyl methacrylamide

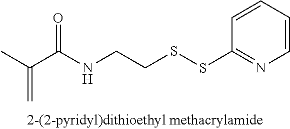 [SMILES: C=C(C)C(=O)NCCSSc1ccccn1]

Group 23′ for binding a signal substance: pyridyl disulfide group (converted to thiol group)
Polymerizable functional group 27: methacryloyl group
First reversible linking group 22: coordinate bond
Second reversible linking group 23: disulphide group

### <3-2> Experimental Procedure

A silica particle modified with a polyhistidine tag and a thiol group made in <1> above was dropped on the base substrate and settled (room temperature, 1 hour) to immobilize the silica particle.

After washing the base plate with pure water, 2-(2-pyridyl)dithioethyl methacrylamide was dissolved in 50 μL of DMSO and diluted with PBS (pH7.4) to prepare a PBS solution comprising 100 μM of 2-(2-pyridyl)dithioethyl methacrylamide. The base plate was immersed in this solution and settled (25° C., overnight) to introduce the methacryloyl group to the silica particle surface by disulfide exchange reaction.

### <4> Synthesis of a Molecularly Imprinted Polymer (Step (1-3))

In accordance with FIG. 12-4, a polymer film of a molecularly imprinted polymer was synthesized.

### <4-1> Reagent and Polymerization Recipe

M28: 2-methacryloyloxyethyl phosphorylcholine (MPC)

TABLE 4

| Reagent | Concentration | |
|---|---|---|
| 2-Methacryloyloxyethyl Phosphorylcholine (MPC) | 50 | mM |
| 2,2′-Bipyridyl | 2 | mM |
| CuBr₂ | 1 | mM |
| L-Ascorbic acid | 0.5 | mM |
| PBS (10 mM phosphate, 140 mM NaCl, pH7.4) | 10 | mL |

### <4-2> Experimental Procedure

In accordance with the polymerization recipe of Table 4, a surface-initiated atom transfer radical polymerization (SI-ATRP) was carried out on the base plate obtained in <3> above to synthesize a polymer thin film of a molecularly imprinted polymer 20a. Specifically, a solution of the above-mentioned polymerization recipe excluding L-ascorbic acid was prepared in a Schlenk flask, and the base plate obtained in <3> above was put in to carry out nitrogen substitution, and then L-ascorbic acid was injected with a syringe to initiate polymerization. Polymerization was carried out for 3 hours at 40° C., and then the base plate was washed with pure water.

### <5> Removal of the Mold (Step (1-4))

In accordance with FIG. 12-5, the mold was cut out from the molecularly imprinted polymer to obtain an analysis sensor 10a-C′ of a detection subject that is base plate-type.

100 mM of EDTA-4Na aqueous solution was dropped to the base plate obtained in <4> above and settled (room temperature, 15 minutes) to remove Cu²⁺. Next, the base plate was washed with pure water and 50 mM of TCEP aqueous solution was dropped and settled (room temperature, 3 hours) to reduce the disulfide bond and achieve conversion to a thiol group (group 23′ for binding a signal substance). Then, the base plate was washed with pure water, immersed in 50 mM of acetate buffer (pH 4.0) containing 0.5 wt % of SDS, and shaken (25° C., overnight) to cut the chelate (coordinate bond) between the NTA-Ni complex on the base plate and the polyhistidine tag on the silica particle, achieve conversion to a Ni-NTA group (group 22′ for binding a specific binding molecule to a target), and remove the silica particle. This causes obtainment of the base material 10a-C′ for making an analysis sensor of a detection subject that is base plate-type.

### <6> Introduction of an Anti-HER2 Affibody and Alexa Fluor 647 (Step (2) and Step (3))

In accordance with FIG. 12-6, Alexa Fluor 647 and an anti-HER2 affibody were introduced to the analysis sensor 10a-C′ of a detection subject that is base plate-type.

### <6-1> Reagent

R43: Alexa Fluor 647 C2-maleimide (Thermo Fisher Scientific)
   Signal group 43: Alexa Fluor 647
   Binding group 23″: maleimide group
R42: Z342-EGFP-Histag
   Specific binding group 42: anti-HER2 affibody
   Binding group 22″: polyhistidine tag
   Different signal group sg: EGFR

The natural Kd for HER2 of the anti-HER2 affibody in Z342-EGFP-Histag is greater than the natural Kd for HER2 of the anti-HER2 affibody where EGFR is not fused due to the effect of the EGFR being fused (i.e., the binding capacity is inferior to the anti-HER2 affibody where EGFR is not fused). In fact, the natural Kd for HER2 of the anti-HER2 affibody in Z342-EGFP-Histag was 1.06×10⁻¹⁰ M.

### <6-2> Experimental Procedure

The base material 10a-C′ for making an analysis sensor of a detection subject that is base plate-type obtained in <5> above was washed with pure water, and 100 μM of Alexa Fluor 647 C₂ Maleimide (in 20% DMSO aqueous solution) was dropped and settled (room temperature, 1 hour) to utilize the thiol group (group 23′ for binding a signal substance) within the molecular imprint recessed part 21 to introduce a fluorescence group Alexa Fluor 647 (signal group 43).

After washing the base plate with pure water, PBS (10 mM phosphate, 140 mM NaCl, pH7.4) solution comprising 0.3 μM of Z342-EGFP-Histag was dropped and settled (room temperature, 1 hour) to utilize the nickel-nitrilotriacetic acid-driven group (group 22′ for binding a specific binding molecule) within the molecular imprint recessed part 21 to introduce an anti-HER2 affibody (specific binding group 42). This causes obtainment of the analysis sensor 10a₁-C of a detection subject that is base plate-type.

<7> Analysis of a Detection Subject with Fluorescence Measurement

The analysis sensor 10a₁-C of a detection subject that is base plate-type obtained in <6> above was used to carry out exosome capturing test with a fluorescence microscope with a SIC automatic dispensing apparatus.

### <7-1> Experimental Procedure

Exosome (exosome secreted by a SK-BR-3 cell) was purified from a SK-BR-3 cell line (high Her2 expression) culture supernatant in the following manner.

A supernatant wherein an SK-BR3 cell was cultured for 3 days at a DMEM culture medium (containing 10% FBS (exosome free), 1% penicillin) was centrifugally separated for 10 minutes at 1500 g and for 10 minutes at 3000 G, which supernatant underwent ultrafiltration (10 minutes at 500 G×2 times). The exosome solution obtained by the ultrafiltration was purified using an exosome extraction kit qEV (Meiwafosis). The particle size and concentration of exosome were measured using q-Nano.

Exosome secreted by a SK-BR-3 cell was dissolved at the concentration of 3.33×10⁻¹⁷ M, 8.33×10⁻¹⁷ M, 1.67×10⁻¹⁶ M, 3.33×10⁻¹⁶ M, 8.33×10⁻¹⁶ M, 1.67×10⁻¹⁵ M, 3.33×10⁻¹⁵ M, and 1.67×10⁻¹⁴ M in PBS (10 mM phosphate, 140 mM NaCl, pH7.4) to prepare an exosome solution. The prepared exosome solution was added to the analysis sensor 10a-C of a detection subject that is base plate-type obtained in <6> above.

Regarding the measurement conditions of the fluorescence microscope, 9 points on the base plate were set as measurement locations, the filter is Cy5, the objective lens is 5-fold, the exposure time is 0.1 second, the power of light is 100%, and the light source is a mercury lamp.

### <7-2> Relationship Between Relative Fluorescence Intensity and Exosome Concentration

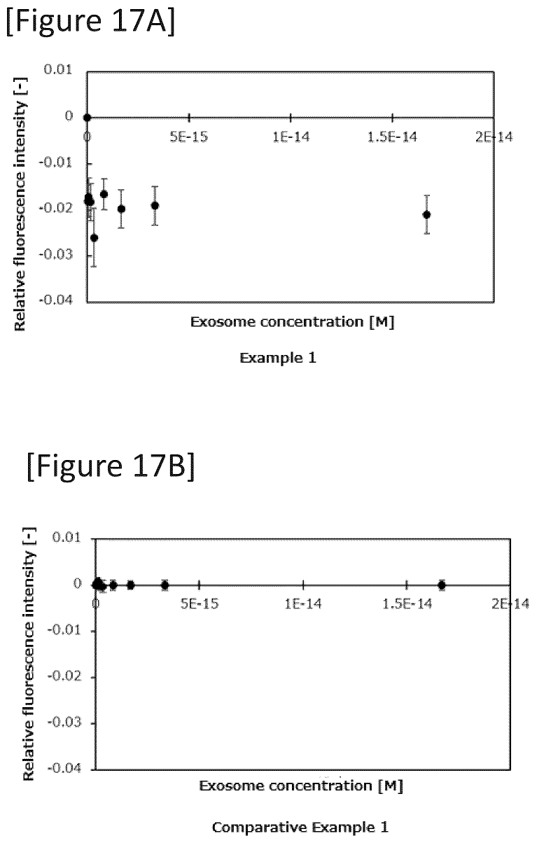

The relative fluorescence intensity was calculated in accordance with the following formula to study the relationship between relative fluorescence intensity and exosome concentration. The result is shown in FIG. 17A.

$$Δ I = \frac{I - {I}_{0}}{{I}_{0}} \qquad [ Numeral 1 ]$$

$$Δ I : \begin{matrix}Relative \\ fluorescence \\ intensity\end{matrix} {I}_{0} : \begin{matrix}Fluorescence intensity of \\ exosome concentration 0\end{matrix}$$

$$I : \begin{matrix}Fluorescence intensity \\ of each concentration\end{matrix}$$

As shown in FIG. 17A, in the analysis sensor 10a₁-C of a detection subject that is base plate-type introduced with Alexa Fluor 647 and anti-HER2 affibody, quenching of about a little less than 2% to a little less than 3% was confirmed at the exosome concentration of 3.33×10⁻¹⁷ M to 1.67×10⁻¹⁴ M. Meanwhile, as discussed in Comparative Example 1 below, in an analysis sensor of a detection subject for comparison where an anti-HER2 affibody is not introduced, there was no change in the fluorescence intensity in all exosome concentrations. In view of the above, it was shown that the analysis sensor 10a-C of a detection subject that is base plate-type where Alexa Fluor 647 and anti-HER2 affibody are introduced detected exosome secreted by a SK-BR-3 cell.

### <7-3> Derivation of Dissociation Constant

Dissociation constant Kd was calculated with curve fitting using DeltaGraph based on the fluorescence intensity change. The calculation formula is shown below.

$$Y = \{ ( 1 + K \times G + K \times H ) - {( {( 1 + K \times G + K \times H )}^{2} - 4 \times K \times K \times H \times G )}^{1 / 2} \} \times \frac{D}{2 KG} \qquad [ Numeral 2 ]$$

$$Y : \begin{matrix}Fluorescence \\ intensity change\end{matrix} H : Exosome concentration$$

$$G : First concentration of host molecule D : \begin{matrix}Maximum fluorescence \\ intensity change\end{matrix}$$

$$K : Binding constant ( provided that it is assumed that G = 0.01 )$$

As a result, the dissociation constant Kd by the analysis sensor 10a₁-C of a detection subject that is base plate-type introduced with Alexa Fluor 647 of the present Example is 6.1×10⁻¹⁸ M, achieving exceptional improvement effect of 8.5 orders with respect to the natural Kd (1.06×10⁻¹⁰) of the anti-HER2 affibody used in the present Example, wherein astonishing binding capacity improvement effect compared to Comparison Example 2 discussed below was recognized. In addition, the binding capacity improvement effect by the present Example was significantly excellent compared to Examples 3 and 5 discussed below.

### Comparative Example 1: Analysis Sensor of a Detection Subject that is Base Plate-Type not Comprising an Affibody

An analysis sensor of a detection subject for comparison was made in the same manner as Example 1 excluding the point of not introducing an affibody. The relationship between the relative fluorescence intensity and exosome concentration was studied in the same manner as Example 1 regarding this analysis sensor of a detection subject for comparison. The result is shown in FIG. 17B.

As shown in FIG. 17B, there was no change in fluorescence intensity in all exosome concentrations in an analysis sensor of a detection subject for comparison where anti-HER2 affibody is not introduced.

### Example 2: Analysis Sensor 10a₁-C of a Detection Subject that is Base Plate-Type (Introducing an Anti-HER2 Affibody as a Specific Binding Substance and Rhodamine as a Signal Substance

In the present Example, an analysis sensor 10a₁-C of a detection subject that is base plate-type (introducing rhodamine as a signal substance) was prepared. In the same manner as Example 1, this analysis sensor of a detection subject that is base plate-type set exosome secreted by an SK-BR-3 cell which is an HER2 overexpress-type breast cancer cell line as the detection subject, set HER2 as the target of the detection subject, and selected and introduced anti-HER2 affibody as a specific binding molecule. In addition, rhodamine was introduced as a signal substance. Furthermore, the obtained analysis sensor of a detection subject that is base plate-type was used to analyze the detection subject.

### <1> Experimental Procedure

The making was carried out with the same method as Example 1, excluding the point of using Tetramethylrhodamine (TAMRA)-5 C2 maleimide instead of Alexa Fluor 647 C2-maleimide as a signal substance R43. In the same manner, a total of three (N=3) analysis sensors 10a₁-C (introducing rhodamine as a signal substance) of a detection subject that are base plate-type were prepared.

### <2> Analysis of a Detection Subject Using Fluorescence Measurement

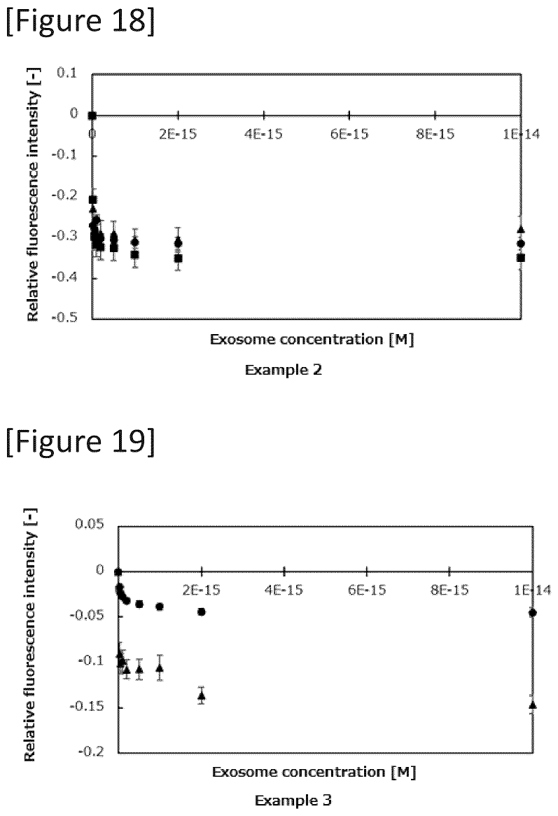

Analysis of exosome secreted by a SK-BR-3 cell was carried out with the same method as Example 1 regarding each of the analysis sensors 10a₁-C (introducing rhodamine as a signal substance, N=3) of a detection subject that are base plate-type obtained in <1> above. Furthermore, the concentrations of the exosome were set as 2.0×10⁻¹⁷ M, 5.0×10⁻¹⁷ M, 1.0×10⁻¹⁶ M, 2.0×10⁻¹⁶ M, 5.0×10⁻¹⁶ M, 1.0×10⁻¹⁵ M, 2.0×10⁻¹⁵ M, and 1.0×10⁻¹⁴ M. Regarding the measurement conditions of the fluorescence microscope, 7 points on the base plate were set as measurement locations, the filter is GW (excitation)-Cy3 (fluorescence), the objective lens is 5-fold, the exposure time is 0.1 second, the power of light is 100%, and the light source is a mercury lamp. The result is shown in FIG. 18.

As a result, in the analysis sensor 10a₁-C of a detection subject that is base plate-type introduced with rhodamine and anti-HER2 affibody, 10-fold or greater quenching was confirmed in all exosome concentrations compared to the analysis sensor 10a₁-C of a detection subject that is base plate-type introduced with Alexa Fluor 647 and anti-HER2 affibody of Example 1, which is recognized as significantly high sensitivity. For example, a little over 30% quenching was observed in the exosome concentration of 1.0×10⁻¹⁴ M. Such sensitivity of the present Example was also excellent compared to Example 3 discussed below. Furthermore, such highly sensitive detection is possible regarding any of N=3, and it was also understood to be excellent in terms of reproducibility. This excellent reproducibility was also significant compared to Example 1 discussed above and Example 3 discussed below.

### <3> Derivation of Dissociation Constant

The dissociation constant Kd was calculated based on the fluorescence intensity change in the same manner as <7> of Example 1. As a result, the dissociation constant Kd by the analysis sensor 10a₁-C of a detection subject that is base plate-type introduced with rhodamine and anti-HER2 affibody of the present Example is 8.41×10⁻¹⁸, achieving exceptional improvement effect of a little less than 9 orders of magnitude with respect to the natural Kd (1.06×10⁻¹⁰) of the anti-HER2 affibody used in the present Example, wherein astonishing binding capacity improvement effect compared to the Comparison Example 2 discussed below was recognized. In addition, the binding capacity improvement effect by the present Example was also significantly excellent compared to Examples 3 and 5 discussed below.

### <4> Derivation of Detection Limit

The detection limit (DL) was calculated in accordance with the following formula.

[Numeral 3]

DL=3.3s/a

s: Standard deviation of fluorescence intensity of exosome concentration 0
a: Inclination by the average fluorescence intensity of concentration 0, 2.0×10⁻¹⁷, 5.0×10⁻¹⁷ [M]

As a result, the detection limit (DL) by the analysis sensor 10a₁-C of a detection subject that is base plate-type introduced with rhodamine and anti-HER2 affibody of the present Example was 5.19×10⁻¹⁷ M, which was also significantly excellent compared to Example 3 discussed below.

### Comparative Example 2: Analysis Sensor of a Detection Subject that is Base Plate-Type Introduced with an Antibody

A sensor for analysis was prepared in the same manner as Example 1 excluding the point of introducing an anti-Her2 antibody (the original dissociation constant with respect to Her2 of this anti-Her2 antibody is 5.8×10⁻¹⁰ M) instead of an anti-HER2 affibody. Specifically, the following procedure was carried out instead of binding Z342-EGFP-Histag to the analysis sensor 10a-C′ of a detection subject that is base plate-type in <6> of Example 1.

100 μM of polyhistidine protein G dissolved in PBS was added to immobilize protein G that can bind an antibody via a polyhistidine tag. 0.3 μM of anti-Her2 antibody dissolved in PBS was added to the base plate to immobilize the anti-Her2 antibody via protein G.

Excluding the point above, a sensor for analysis was made in the same manner as Example 1, and the dissociation constant was derived in the same manner as <3> of Example 2. As a result, the dissociation constant Kd by the sensor for analysis of the present Comparative Example is 6.34×10⁻¹⁶ M, which remained an improvement effect of 6 orders with respect to the original Kd (5.8×10⁻¹⁰) of the anti-HER2 antibody used in the present Comparative Example.

### Example 3: Analysis Sensor of a Detection Subject that is Base Plate-Type (Provided that the Sensor is a Type of Analysis Sensor 101 of a Detection Subject that is Base Plate-Type that Utilizes a Signal Substance Fused to a Specific Binding Group

In the present Example, an analysis sensor of a detection subject that is base plate-type (provided that the sensor is a type of analysis sensor 101 of a detection subject that is base plate-type that utilizes a signal substance fused to a specific binding group) was made. In the same manner as Example 1, this analysis sensor of a detection subject that is base plate-type set exosome secreted by an SK-BR-3 cell which is an HER2 overexpress-type breast cancer cell line as the detection subject, set HER2 as the target of the detection subject, and selected and introduced anti-HER2 affibody as a specific binding molecule. In addition, a signal substance fused to a specific binding group without introducing a signal substance was utilized. Furthermore, analysis of the detection subject was carried out using the obtained analysis sensor of a detection subject that is base plate-type.

### <1> Experimental Procedure

An analysis sensor of a detection subject that is base plate-type was made in the same manner as Example 3 (N=2) excluding the step of introducing Tetramethylrhodamine-5 C2 maleimide. In other words, the analysis sensor of the detection subject that is base plate-type of the present Example is not introduced with a signal substance, unlike the analysis sensor 10a-C of a detection subject that is base plate-type of Example 2. In this regard, since Z342-EGFP-Histag (a molecule having a polyhistidine tag and an anti-HER2 affibody to which a fluorescence group EGFP is fused) is used as the specific binding molecule R42 that provides the specific binding group 42 as discussed above (the column of Example 1) in this sensor, strictly speaking, the specific binding group 42 is an anti-HER2 affibody to which a fluorescence group EGFP is fused, i.e., the specific binding group 44 wherein a signal group 43 is conjugated with the specific binding group 42. Therefore, the analysis sensor of a detection subject that is base plate-type of the present Example is a type of analysis sensor 101 of a detection subject that is base plate-type utilizing a signal substance fused to a specific binding group.

### <2> Analysis of a Detection Subject Using Fluorescence Measurement

Regarding each of the analysis sensors (utilizing EGFP fused to an anti-HER2 affibody) of a detection subject that are base plate-type obtained in <1> above, analysis of exosome secreted by an SK-BR-3 cell was carried out with the same method and condition as Example 2 (provided that the filter was changed to BW (excitation)-Cy3 (fluorescence) to measure EGFP, N=2). The result is shown in FIG. 19.

As a result, in a sensor that is a type of sensor 101 of a detection subject that is base plate-type utilizing EGFP fused to an anti-HER2 affibody, while the fluctuation range was about 5% to 15% with the quenching at the exosome concentration of 1.0×10⁻¹⁴ M, exosome was detected with high sensitivity.

### <3> Derivation of Dissociation Constant

The dissociation constant Kd was calculated in the same manner as <7> of Example 1 based on the fluorescence intensity change. As a result, the dissociation constant Kd is 2.29×10⁻¹⁷, which was an exceptional improvement with respect to the original Kd (1.06×10⁻¹⁰) of the anti-HER2 affibody to which a fluorescence group EGFP is fused.

### <4> Derivation of Detection Limit

The detection limit (DL) was calculated in the same manner as Example 2. As a result, the detection limit (DL) was 3.70×10⁻¹⁴ M.

### Example 4: Analysis Sensor 10-PC of a Detection Subject that is Particle-Like and Immobilized on a Base Plate (Introducing an Anti-HER2 Affibody as a Specific Binding Substance

In the present Example, an analysis sensor 10-PC of a detection subject that is particle-like and immobilized on a base plate was prepared. This analysis sensor of a detection subject that is particle-like and immobilized on a base plate set exosome secreted by an SK-BR-3 cell which is an HER2 overexpress-type breast cancer cell line as the detection subject, set HER2 as the target of the detection subject, and selected and introduced an anti-HER2 affibody as a specific binding molecule. Furthermore, analysis of the detection subject was carried out using the obtained analysis sensor 10-PC of a detection subject that is particle-like and immobilized on a base plate.

Regarding a method of making an analysis sensor of a detection subject that is particle-like and immobilized on a base plate, a base plate 10-PC′ for making an analysis sensor of a detection subject that is base plate-type was made in accordance with FIG. 13-1b, FIG. 13-2, FIG. 13-3, and FIG. 15, and the analysis sensor 10-PC of a detection subject was made in accordance with FIG. 13-4.

### <1> Synthesis of a Disulfide Bond-Type Methacryloyl Modification Reagent

In accordance with the following scheme, a disulfide bond-type methacryloyl modification reagent (Compound (5) of the following scheme) used in <2> discussed below was synthesized. In the present Example, this reagent was used as a monomer M222 comprising a binding group BG22′ and a first reversible linking group 22 polymerizable functional group 27.

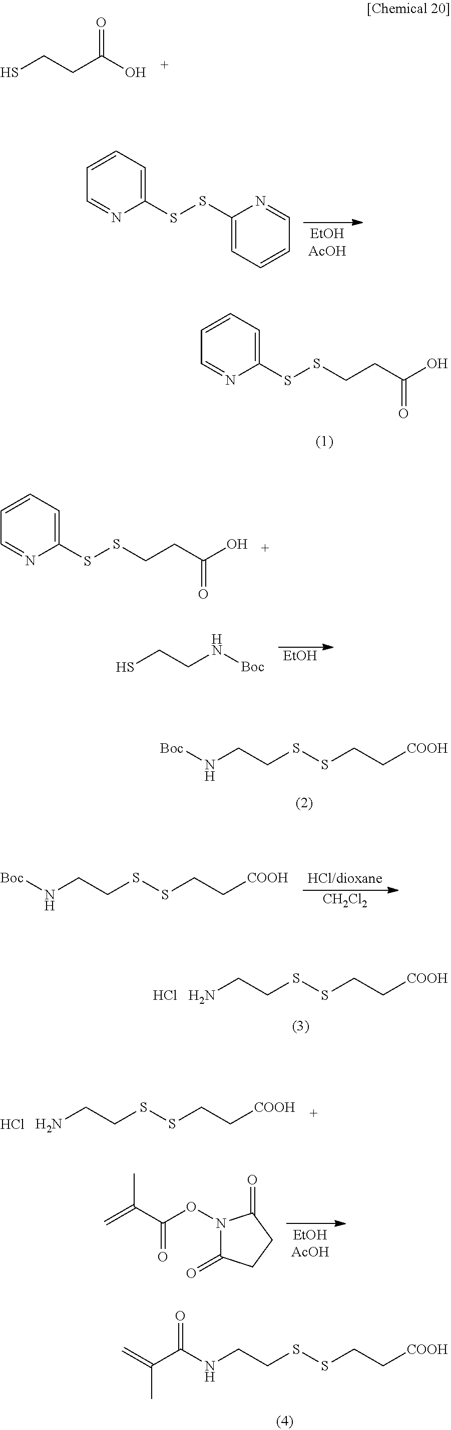

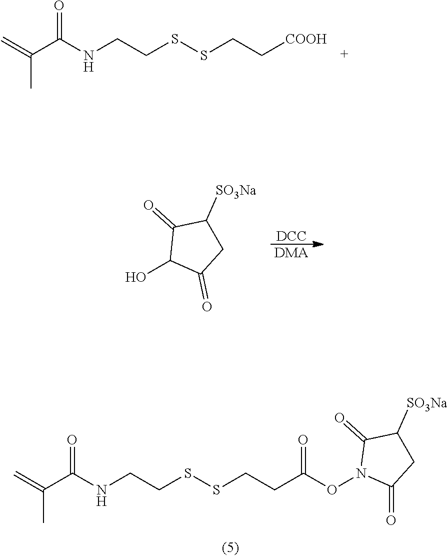

3-mercaptopropionic acid (0.900 g, 8.50 mmol) was dissolved in 20 mL of ethanol to add 1.6 mL of acetate. 2,2-dipyridyl disulfide (3.75 g, 17 mmol) dissolved in 30 ml of ethanol was dropped thereto while stirring at room temperature. Reaction was carried out for 3 hours at 25° C. to obtain Compound (1).

The purified Compound (1) (1.43 g, 3.97 mmol) was dissolved in ethanol 15 mL, and then 2-(Boc-amino) ethanethiol (1.69 mL, 9.99 mmol, 2 eq) dissolved in 20 mL of ethanol was added. Reaction was carried out for 3 hours at room temperature to obtain Compound (2).

4N HCl dioxane (6.60 mL, 25.5 mmol, 5 eq) dissolved in 10 mL of CH₂Cl₂ was dropped to the purified compound (2) (1.43 g, 5.10 mmol) dissolved in 5 mL of CH₂Cl₂ while being stirred under ice cooling. Then, reaction was carried out overnight at room temperature to obtain Compound (3).

The purified Compound (3) (531.6 mg, 2.45 mmol) was dispersed in CH₂Cl₂ 5 mL to add DIEA (1.28 mL, 7.35 mmol, 3 eq). 3.5 mL of CH₂Cl₂ that dissolved N-succinimidyl methacrylate (670 mg, 3.68 mmol, 1.5 eq) was dropped thereto in a nitrogen atmosphere to be reacted overnight at room temperature to obtain Compound (4).

The purified Compound (4) (290 mg, 1.16 mmol, 1.1 eq), sulfo-NHS (230 mg, 1.1 mmol), and DCC (304.5 mg, 1.54 mmol, 1.4 eq) were dissolved in DMA 5 mL to be reacted for 24 hours at room temperature. After the reaction, the solution was cooled at 4° C. and precipitate was removed by filtration. Then, vacuum distillation of the filtrate was carried out to remove DMA. The obtained white-colored individual was dissolved in AcOEt, to which Hexane was added to cause another precipitation to obtain Compound (5). (¹H-NMR chart 7 (500 MHz, CD₃OD): δ=8.1 (t, 1H, —CO—NH—C—), 5.7-5.3 (s, 2H, CH3-C═CH₂), 4.0-3.8 (d, 1H, —CO—CH—SO₃Na), 3.4-3.3 (m, 2H, NaSO₃—CH—CH₂—), 3.3-2.7 (m, 8H, —NH—CH₂—CH₂—S—S—CH₂—CH₂—), 1.85 (s, 3H, CH₃—C═CH₂))

### <2> Modification of a Mold (Step (1-11))

In accordance with FIG. 13-1b, a mold HSA modified with a methacryloyl group via a disulfide bond was made.

### <2-1> Reagent

Albumin, from Human Serum (HSA; human serum albumin) (WAKO JUNYAKU KOGYO)
   Mold 60: HSA
   Binding group BG22″: amino group
M222: Compound (5) synthesized in <1> above
   Binding group BG22′: carboxylic acid-active ester group
   First reversible linking group 22: disulfide group
   Polymerizable functional group 27: methacryloyl group

### <2-2> Experimental Procedure

2 mL of 10 mM phosphate buffer solution (pH 7.4) that dissolved HSA (100 mg, 2.23 μmol) and 1 mL of 10 mM phosphate buffer solution (pH 7.4) that dissolved Compound (5) synthesized in <1> above (20 mg, 44.6 μmol, 20 eq) were mixed to be reacted overnight at 4° C. Then, washing was carried out 3 times with Amicon Ultra-4 10 kDa (4° C., 7500 G, 20 min) to obtain a mold HSA modified with a methacryloyl group via a disulfide bond.

### <3> Synthesis of a Molecularly Imprinted Polymer (Step (1-12))

In accordance with FIG. 13-2, a particle of a molecularly imprinted polymer was synthesized.

### <3-1> Reagent and Polymerization Recipe

M28: 2-methacryloyloxyethyl phosphorylcholine (MPC)
M29: N-isopropylacrylamide (NIPAm)

TABLE 5

| Reagent | Amount of use |
|---|---|
| Methacryloyl group modification HSA | 18.3 mg (0.30 μmol) |
| N-Isopropylacrylamide (NIPAm) | 610 mg (5.40 mmol) |
| 2-Methacryloyloxyethyl Phosphorylcholine (MPC) | 88.5 mg (0.30 mmol) |
| Pyrrolidyl acrylate (PyA) | 63.6 mg (0.445 mmol) |
| N,N′-Methylenebisacrylamide (MBAA) | 46.2 mg (0.30 mmol) |
| 2-2′-Azobis(2-methyl propionamide) dihydrochloride (V-50) | 316 mg (1.20 mmol) |
| PBS (10 mM phosphate, 140 mM NaCl, pH7.4) | 50 mL |

### <3-2> Experimental Procedure

In accordance with the polymerization recipe of Table 5, emulsifier-free precipitation polymerization was carried out for 16 hours at 70° C. in a nitrogen atmosphere in a 50 mL Schlenk flask to synthesize particle MIP-NGs of a molecularly imprinted polymer 20.

### <4> Removal of Mold (Step (1-13))

In accordance with FIG. 13-3, the mold was removed from the molecularly imprinted polymer.

Regarding a PBS liquid comprising a composite of the particle MIP-NGs of the molecularly imprinted polymer 20 and the mold HSA, pure water substitution of a solvent was carried out by ultrafiltration (25° C., 7500 g, 20 minutes, 3 times) using a dialysis tube of Amicon Ultra-4 10 kDa. Tris(2-carboxyethyl)phosphine Hydrochloride (TCEP) was added to this composite so as to be 20 mM to be reacted overnight at 25° C. This causes the disulfide bond between the mold HSA and MIP-NGs to be reduced and cleaved, exposing the thiol group (group 22′ for binding a specific binding molecule to a target).

Furthermore, screening was carried out using a chromatography filling agent Sephadex G-100 for size exclusion chromatography, removal of TCEP was carried out by ultrafiltration (25° C., 7500 g, 20 min, 3 times) using a dialysis tube of Amicon Ultra-4 10 kDa, and solvent substitution to 140 mM NaCl 10 mM tris-hydrochloric acid buffer pH 7.4 was carried out. Furthermore, the mold HSA was removed by anion exchange chromatography. This caused obtainment of the particle MIP-NGs of the molecularly imprinted polymer 20.

### <5> Particle Analysis

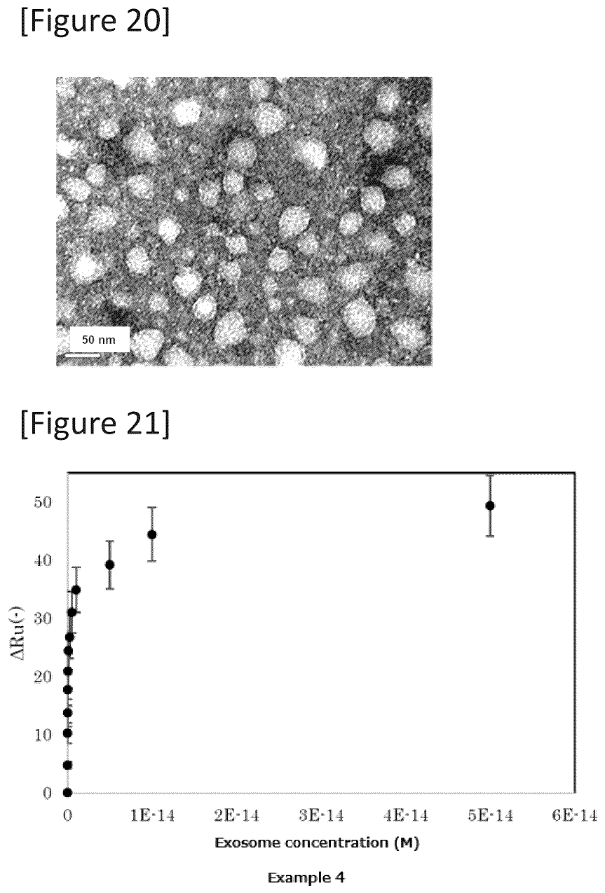

Regarding the obtained MIP-NGs, dynamic light scattering (DLS) measurement and transmission electron microscope (TEM) measurement were carried out. In the TEM measurement, the average value of the long axis and the short axis of the particle was set as a particle size in the transmission electron microscopic image, and said particle sizes of 100 randomly selected particles were measured to use the average value thereof as the number for average particle size. The result is shown in the table below. In addition, the TEM image is shown in FIG. 20. In FIG. 20, the length of the scale bar is 50 nm.

TABLE 6

| DLS | | | | TEM |
|---|---|---|---|---|
| | Volume | | | Number |
| Z average | average | | Solid | average |
| particle | particle | | component | particle |
| size | size | PDI | concentration | size |
| 128.6 nm | 156.4 nm | 0.293 | 0.04 | 50-80 nm |

### <6> Immobilization of a Particle to a Base Plate

In accordance with FIG. 15 (provided that the molecularly imprinted polymer 20 is replaced with the molecularly imprinted polymer 20), the particle MIP-NGs of the molecularly imprinted polymer 20 obtained in <4> above was immobilized on the base plate.

After washing the SPR gold base plate with pure water and ethanol, the SPR gold base plate was immersed in an ethanol solution prepared so that 11-Mercaptoundecanoic acid would be 1 mM to be incubated overnight at 25° C. After washing the base plate after the reaction with ethanol and water and drying the base plate with nitrogen gas, Biacore3000 was used to carry out injection of a PBS solution of 0.1 M N-Hydroxysuccinimide (NHS), 0.4 M 1-(3-dimethylaminopropyl)-3-ethylcarbodiimide hydrochloride (EDC) for incubation for 10 minutes at 25° C. The obtained base plate (wherein the base plate surface is modified with a carboxylic acid-active ester group as the binding group BG9″) was injected with 400 μg/mL of MIP-NGs solution to be incubated for 10 minutes at 25° C. to obtain a base plate wherein MIP-NGs is fixed. This causes obtainment of the base material 10-PC′ for making a sensor for analyzing a detection subject.

### <7> Introduction of Affibody (Step (2))

In accordance with FIG. 13-4, an anti-Her2 affibody was introduced to the base material 10-PC′ for making a sensor for analyzing a detection subject obtained in <6> above.

### <7-1> Reagent

Anti-Her2 Affibody® Molecule (anti-Her2 affibody molecule, Abcam; the natural Kd of this molecule is 3.2×10⁻¹¹ M, and the molecular weight is about 6,000)
Dithiothreitol
   Specific binding group 42: anti-Her2 affibody
   Binding group 22″: thiol group

### <7-2> Experimental Procedure

10 μL of a 1 mg/mL solution of anti-Her2 affibody molecule (10 mM PB, 137 mM NaCl, 0.05 vol % Tween20 (pH 7.4); PBST) was added to 40 μL of 20 mM of DTT solution (50 mM PB, 137 mM NaCl (pH7.5)), reacted for 2 hours at 25° C., then Amicon Ultra-0.5 (MWCO: 10 kDa) was used to carry out ultrafiltration (4° C., 14000×g, 15 minutes) 3 times to carry out purification at PBST. This causes obtainment of a molecule R42 that provides anti-Her2 affibody.

The base material 10-PC′ for making a sensor for analyzing a detection subject obtained in <6> above was immersed in 3.1 mM of 2,2-dipyridyl disulphide solution (ethanol:water=1:1 (volume ratio)) for incubation for 3 hours at 25° C. to activate the thiol group (group 22′ for binding a specific binding molecule to a target) within the molecular imprint recessed part 21 into a pyridyl disulfide group (group 22′ for binding a specific binding molecule to a target). Then, the pyridyl disulfide group was injected with 10 μg/mL of solution of the molecule R42 that provides an anti-Her2 affibody for 7 minutes at the flowrate of 20 μL/min to bind the anti-Her2 affibody group (specific binding group 42). Then, 1 mg/mL skim milk aqueous solution and 1 M aminoethanol (pH8.4) were reacted in order, to carry out blocking treatment of the base plate surface (binding group BG9″ that was not used to fix a particle). This caused obtainment of the sensor 10-PC for analyzing a detection subject.

### <8> Analysis of a Detection Subject by SPR Measurement

SPR measurement was carried out using the sensor 10-PC for analyzing a detection subject that is particle-like and immobilized at a base plate obtained in <7> above.

### <8-1> Experimental Procedure

Exosome secreted by an SK-BR-3 cell was dissolved in PBS (10 mM phosphate, 140 mM NaCl, pH7.4) at the concentration of 0×10⁻¹⁶ M, 0.032×10⁻¹⁶ M, 0.064×10⁻¹⁶ M, 0.125×10⁻¹⁶ M, 0.25×10⁻¹⁶ M, 0.5×10⁻¹⁶ M, 1.0×10⁻¹⁶ M, 2.5×10⁻¹⁶ M, 5.0×10⁻¹⁶ M, 10×10⁻¹⁶ M, 50×10⁻¹⁶ M, 100×10⁻¹⁶ M, 500×10⁻¹⁶ M, 1000×10⁻¹⁶ M to prepare an exosome solution. The prepared exosome solution was added to the sensor 10-PC for analyzing a detection subject that is particle-like and immobilized on a base plate obtained in <7> above.

The measurement conditions of SPR are as shown below.
Flowrate: 20 μL/min
Injection volume: 100 μL
Temperature: 25° C.
Data point: the point of 6 minutes after injection
Running buffer: PBS

### <8-2> Result

The difference in the resonance unit (RU) value between right before the injection of the exosome solution and at the point of 6 minutes after the injection was used as the amount of change in response to create an adsorption isotherm. The result thereof is shown in FIG. 21. Furthermore, the dissociation constant Kd was calculated by curve fitting in the same manner as <7> of Example 1. As a result, the dissociation constant Kd is 2.0×10⁻¹⁸ (M), which was an exceptional improvement with respect to the natural Kd (3.2×10⁻¹¹ M) of the anti-HER2 affibody used in the present Example.

### Example 5: Sensor 10b-PC of a Detection Subject that is Particle-Like and Immobilized on a Base Plate (Introducing Anti-HER2 Affibody as a Specific Binding Substance and ATT0647N as a Signal Substance

In the present Example, a sensor 10b-PC of a detection subject that is particle-like and immobilized on a base plate was made. This analysis sensor of a detection subject that is particle-like and immobilized on a base plate set exosome secreted by an SK-BR-3 cell which is an HER2 overexpress-type breast cancer cell line as the detection subject, set HER2 as the target of the detection subject, and selected and introduced anti-HER2 affibody as a specific binding molecule. In addition, ATT0647N was introduced as a signal substance. Furthermore, analysis of the detection subject was carried out using the obtained analysis sensor 10b-PC of a detection subject that is particle-like and immobilized on a base plate.

Regarding the analysis sensor 10b-PC of a detection subject that is particle-like and immobilized on a base plate, since the analysis sensor 10c-P of a detection subject that is particle-like is immobilized on the base plate, the sensor is an embodiment wherein the interaction group 25 is excluded. Therefore, regarding a method of making the analysis sensor 10b-PC of a detection subject that is particle-like and immobilized on a base plate, a base plate 10b-PC′ for making an analysis sensor of a detection subject that is particle-like was made in accordance with FIG. 14-1, FIG. 14-2 (provided that a monomer M25 having the interaction group 25 is not used), FIG. 14-3, FIG. 15, and FIG. 14-4, and the analysis sensor 10b-PC of a detection subject was made in accordance with FIG. 14-5.

### <1> Modification of Mold (Step (1-21))

In accordance with FIG. 14-1, a mold HSA modified with a methacryloyl group via a disulfide bond was made.

### <1-1> Reagent

Albumin, from Human Serum (HSA; human serum albumin) (WAKO JUNYAKU KOGYO)
   Mold 60c: HSA
   Binding group BG6″: amino group
M27: Compound (5) synthesized in <1> of Example 4
   Binding group BG6′: carboxylic acid-active ester group
   Third reversible linking group 26b: disulfide group
   Polymerizable functional group 27: methacryloyl group

### <1-2> Experimental Procedure

2 mL of 10 mM phosphate buffer solution (pH 7.4) wherein HSA (100 mg, 2.23 μmol) is dissolved and 1 mL of phosphate buffer solution (pH 7.4) wherein Compound (5) synthesized in <1> of Example 4 (20 mg, 44.6 μmol, 20 eq) is dissolved were mixed to be reacted overnight at 4° C. Then, washing was carried out with Amicon Ultra-4 10 kDa (4° C., 7500 G, 20 min) 3 times to obtain a mold HSA modified with a methacryloyl group via a disulfide bond.

### <2> Synthesis of a Molecularly Imprinted Polymer (Step (1-22))

In accordance with FIG. 14-2 (provided that a monomer M25 having the interaction group 25 was not used), a particle of a molecularly imprinted polymer was synthesized.

Specifically, emulsifier-free precipitation polymerization was carried out for 16 hours at 70° C. in a nitrogen atmosphere in a 50 mL Schlenk flask in accordance with the same polymerization recipe as Table 5 of Example 4 to synthesize particle MIP-NGs of a molecularly imprinted polymer 20b (in the form of not having the interaction group 25 of the molecularly imprinted polymer 20c).

### <3> Removal of Mold (Step (1-23))

In accordance with FIG. 14-3 (provided that the interaction group 25 is absent), a mold was removed from a molecularly imprinted polymer.

Regarding a PBS liquid comprising a composite of a particle MIP-NGs of the molecularly imprinted polymer 20b (in the form of not having the interaction group 25 of the molecularly imprinted polymer 20c) and the mold HSA, pure water substitution of a solvent was carried out by ultrafiltration (25° C., 7500 g, 20 minutes, 3 times) using a dialysis tube of Amicon Ultra-4 (10 kDa). Tris(2-carboxyethyl)phosphine Hydrochloride (TCEP) was added to this composite so as to become 20 mM to be reacted overnight at 25° C. This caused the disulfide bond between the mold HSA and MIP-NGs to be reduced and cut to expose the thiol group (binding group 26b′).

Furthermore, screening was carried out using a chromatography filling agent Sephadex G-100 for size exclusion chromatography, removal of TCEP was carried out by ultrafiltration (25° C., 7500 g, 20 min, 3 times) using a dialysis tube of Amicon Ultra-4 10 kDa, and solvent substitution to 140 mM NaCl, 10 mM tris-hydrochloric acid buffer pH 7.4 was carried out. Furthermore, the mold HSA was removed by anion exchange chromatography. This caused obtainment of the particle MIP-NGs of the molecularly imprinted polymer 20 (in the form of not having the interaction group 25 of the molecularly imprinted polymer 20c).

### <4> Fixing of a Particle to a Base Plate

In accordance with FIG. 15 (provided that the particle-like molecularly imprinted polymer 20c is replaced with the particle-like molecularly imprinted polymer 20b), the particle MIP-NGs of the molecularly imprinted polymer 20b (in the form of not having the interaction group 25 of the molecularly imprinted polymer 20c) obtained in <3> above was immobilized on the base plate. The specific Experimental procedure is the same as <6> of Example 4.

### <5> Synthesis of a Functional Molecule R24

The following scheme was used to synthesize a functional molecule R24 having a functional group 24b and a group 22b′ for binding a specific binding molecule to a target, which is used in <6> discussed below. The functional molecule R24 is a compound represented by Formula (7a) above.

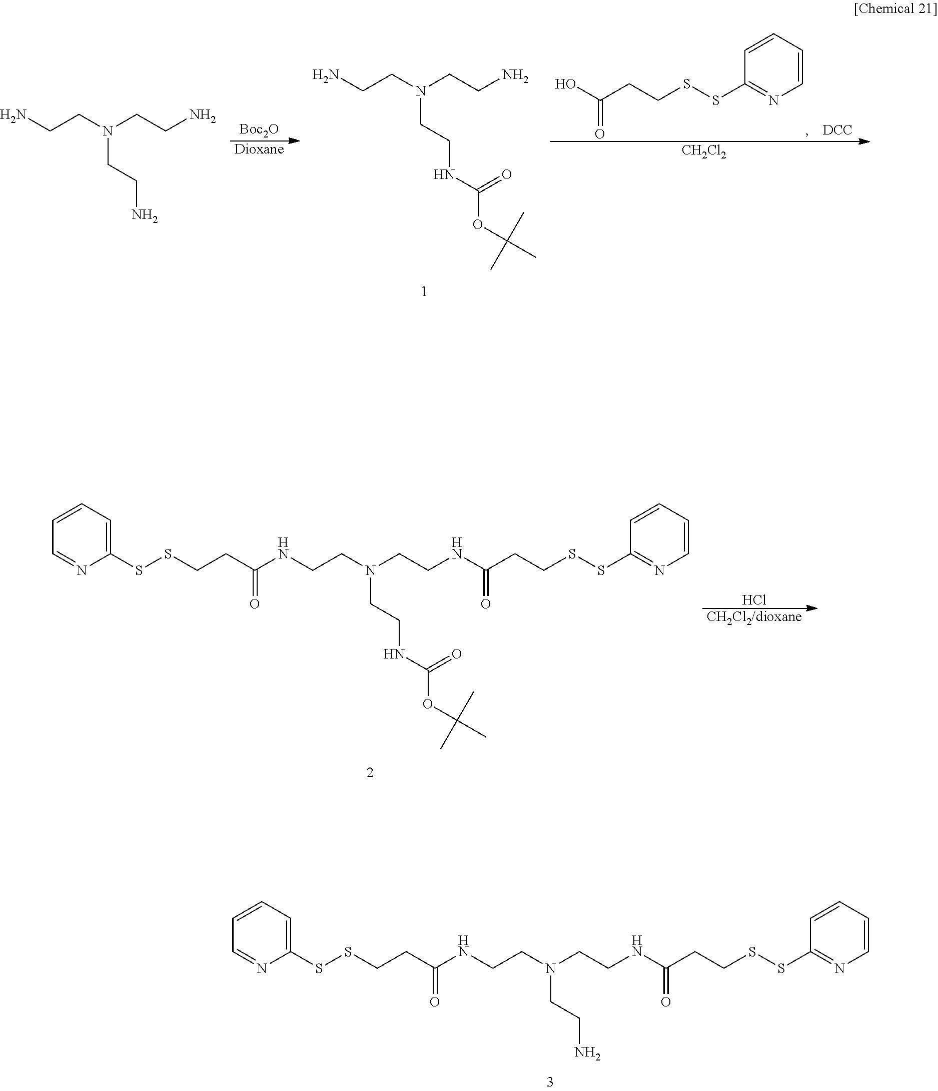

Tris(2-aminoethyl)amine 1800 μL (12 mmol) was dissolved in Dioxane (4 mL) to be stirred under ice cooling. dioxane (10 mL), where Boc₂O 440 mg (2.0 mmol) was dissolved, was slowly dropped thereto to be further stirred overnight (0° C.→room temperature). Vacuum distillation of the solvent after the reaction was carried out then, the residue was dissolve with Milli-Q water to carry out extraction with CH₂Cl₂ (15 mL) (×4). The organic layer was dried with Na₂SO₄, then the solvent was removed by using a rotary evaporator, and vacuum drying was carried out to obtain a clear oil-like targeted object (1). (yield: 364 mg, 74%) ¹H-NMR (300 MHz, CDCl₃) 5=5.29 (br, 1H, carbamide), 3.18 (t, 2H, —CH₂—NH-Boc), 2.75 (t, 4H, H₂N—CH₂—), 2.53 (m, 6H, —CH₂—), 1.45 (s, 9H, Boc)

(3-Pyridyldithio)propionic acid 323 mg (1.5 mmol) and DCC 330 mg (1.6 mmol) were dissolved in CH₂Cl₂ (10 mL) to be stirred under ice cooling. Compound (1) 130 mg (0.53 mmol) was added to be further stirred for 1 hour. After the reaction, the precipitate was removed by filtration, and the solvent underwent vacuum distillation with a rotary evaporator. The residue was purified with silica gel chromatography (EA/Hx=45/55→100/0, EA/MeOH=99/1→80/20) to obtain Compound (2). (yield: 230 mg, 68%) ¹H-MMR (300 MHz, CDCl₃) 5=8.44 (d, 2H, pyridyl), 7.70-7.60 (m, 4H, pyridyl), 7.18 (br, 2H, amide), 7.12-7.07 (m, 2H, pyridyl), 5.01 (br, 1H, carbamide), 3.34-3.29 (m, 4H, H₂N—CH₂—), 3.14-3.07 (m, 6H, —CH₂—NH-Boc, —CH₂—), 2.68 (t, 4H, —CH₂—), 2.61-2.51 (m, 6H, —CH₂—), 1.43 (s, 9H, Boc)

Compound (2) 230 mg (0.36 mmol) was dissolved in CH₂Cl₂ (5 mL), to which 4 N HCl in dioxane 0.5 mL was added to be stirred under ice cooling. After being stirred overnight (0° C.→room temperature), an excessive amount of diethyl ether is added to separate precipitate with decantation. The obtained solid was washed with diethyl ether, which underwent vacuum drying to obtain a targeted object (Compound (3)). (yield: 255 mg, quant.) ¹H-NMR (300 MHz, D₂O) 5=8.51 (d, 2H, pyridyl), 8.14 (t, 2H, pyridyl), 8.01 (d, 2H, pyridyl), 7.56 (m, 2H, pyridyl), 3.67-3.3.56 (m, 6H, HN—CH₂—), 3.43 (t, 6H, —CH₂—N), 3.09 (t, 4H, —CH₂—), 2.74 (t, 4H, —CH₂—)

### <6> Introduction of a Function Group 24b (Step (1-24))

In accordance with FIG. 14-4 (provided that the interaction group 25 is absent), the functional molecule R24 was introduced to the binding group 26b″ of the molecular imprint recessed part 21c (provided that the interaction group 25 is absent).

### <6-1> Reagent

Functional molecule R24: the compound represented by Formula (7a) above obtained in <5> above.
   Group 22b′ for binding a specific binding molecule: pyridyl disulfide group
   Group 23b′ for binding a signal substance: amino group
   Binding group 26b″: pyridyl disulfide group

### <6-2> Experimental Procedure

The base plate wherein particle MIP-NGs of the molecularly imprinted polymer 20b (in the form of not having the interaction group 25 of the molecularly imprinted polymer 20c) is immobilized that is obtained in <4> above was immersed in a 900 μg/mL PBS solution of the functional molecule R24 to react the pyridyl disulfide group (binding group 26b″) of the binding molecule R24 and the thiol group (binding group 26b′) of the molecularly imprinted polymer 20b for 2 hours at room temperature ° C. This caused obtainment of the base material 10b-PC′ for making a sensor for analyzing a detection subject.

### <7> Introduction of an Affibody and a Fluorescence Substance (Step (2) and Step (3))

In accordance with FIG. 14-5 (provided that the interaction group 25 is absent), an anti-Her2 affibody and a fluorescence substance were introduced to the base material 10b-PC′ for making a sensor for analyzing a detection subject.

### <7-1> Reagent

Molecule R42 that provides an anti-Her2 affibody: the compound synthesized in <7-2> of Example 4 using Anti-Her2 Affibody® Molecule (anti-Her2 affibody, Abcam) and dithiothreitol
   Specific binding group 42: anti-Her2 affibody (the natural Kd of this molecule is 3.2×10⁻¹¹ M, and the molecular weight is about 6,000)
   Binding group 22b″: thiol group
Signal substance R43: Atto647N-NHS (ATTO-TEC)
   Signal group 43: Atto647N
   Binding group 23b″: carboxylic acid-active ester group

### <7-2> Experimental Procedure

The 10 μg/mL PBS solution of the molecule R42 that provides the anti-Her2 affibody was reacted with the base material 10b-PC′ for making a sensor for analyzing a detection subject obtained in <7> above (room temperature, 2 hours) to introduce the anti-Her2 affibody (specific binding group 42) to the pyridyl disulfide group (group 22b′ for binding a specific binding molecule). Then, 100 μM of 2-mercaptoethanol solution was reacted to carry out capping of the remaining pyridyl disulfide group (remaining group of the group 22b′ for binding a specific binding molecule). Next, the base plate was reacted with an Atto647N-NHS solution (room temperature, 2 hours) to introduce Atto647N (signal group 43) on the amino group (group 23b′ for binding a signal substance). This caused obtainment of the sensor 10b-PC for analyzing a detection subject (wherein the sensor 10c-P for analyzing a detection subject with no interaction group 25 is immobilized on a base plate).

### <8> Analysis of a Detection Subject by Fluorescence Measurement

Exosome capturing test using a fluorescence microscope with a SIC automatic dispensing apparatus was carried out using the sensor 10b-PC for analyzing a detection subject obtained in <7> above (wherein the sensor 10c-P for analyzing a detection subject with no interaction group 25 is immobilized on a base plate).

### <8-1> Experimental Procedure

Exosome secreted by an SK-BR-3 cell was dissolved in PBS (10 mM phosphate, 140 mM NaCl, pH7.4) at the concentration of 0 to 1.0×10⁻¹³ M to prepare an exosome solution. The prepared exosome solution was dropped to the sensor for analyzing a detection subject obtained in <7> above.

Regarding the measurement conditions of the fluorescence microscope, the measurement location is point 3 on the base plate, the filter is Cy5, the objective lens is 5-fold, the exposure time is 0.1 second, the power of light is 100%, and the light source is a mercury lamp.

### <8-2> Result

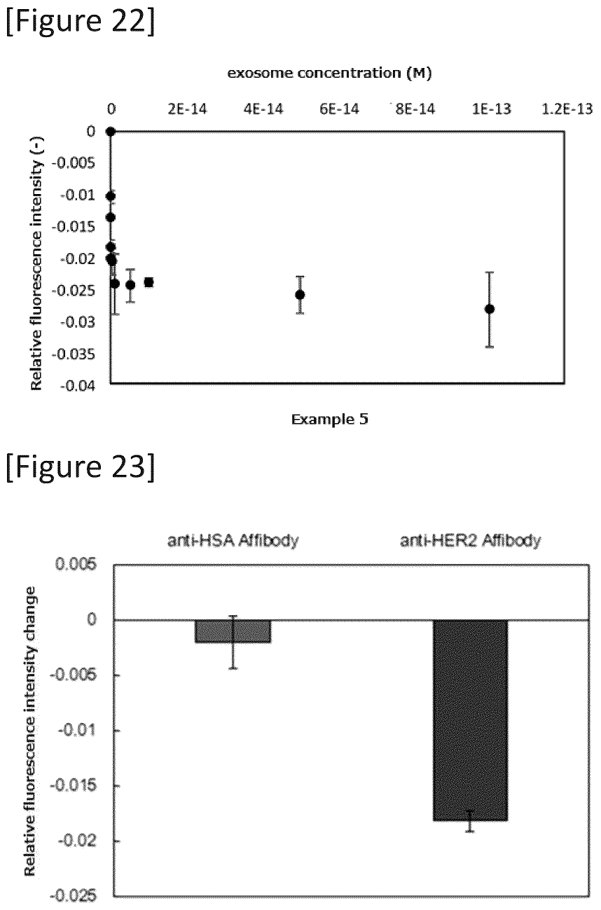

In the same manner as Example 1, the relative fluorescence intensity was calculated to study the relationship between the relative fluorescence intensity and exosome concentration. The result is shown in FIG. 22.

As shown in FIG. 22, in the sensor 10b-PC for analyzing a detection subject obtained in <7> above (wherein the sensor 10c-P for analyzing a detection subject with no interaction group 25 is immobilized on a base plate), it has been confirmed that the degree of quenching increases in an exosome concentration-dependent manner. In other words, the recognition capacity with respect to an exosome secreted by an SK-BR-3 cell was shown.

The dissociation constant Kd was calculated by curve fitting in the same manner as <7> of Example 1. As a result, the dissociation constant Kd was 1.0×10⁻¹⁷ (M), which was exceptional improvement with respect to the natural Kd (3.2×10⁻¹¹ M) of the anti-HER2 affibody molecule used in the present Example. In addition, detection limit was derived in the same manner as <4> of Example 2. As a result, the detection limit was 5.93×10⁻¹⁸ (M).

Another sensor 10b-PC for analyzing a detection subject (wherein the sensor 10c-P for analyzing a detection subject with no interaction group 25 is immobilized on a base plate) was obtained in the same manner as described above excluding the point of using an anti-HSA affibody instead of the anti-HER2 affibody. FIG. 23 shows the relative fluorescence intensity change of when exosome (5×10⁻¹⁷ M) secreted by an SK-BR-3 cell which is an HER2 overexpress-type breast cancer cell line was added to the sensor for analysis of the present Example, wherein the anti-HER2 affibody was introduced and a different sensor for analysis where the anti-HSA affibody was introduced.

As shown in FIG. 23, the exosome secreted by an SK-BR-3 cell was specifically detected by the sensor for analysis of the present Example where the anti-HER2 affibody was introduced.

### Example 6: Analysis Sensor 10a-C of a Detection Subject that is Base Plate-Type (Introducing Anti-HER2 Nanobody as a Specific Binding Substance and Alexa Fluor 647 as a Signal Substance

An analysis sensor 10a-C of a detection subject that is base plate-type (introducing Alexa Fluor 647 as a signal substance) was made in the same manner as Example 1 excluding the point of introducing an anti-Her2 nanobody instead of the anti-HER2 affibody. The original Kd of the anti-Her2 nanobody molecule is 1×10⁻⁹ M.

The dissociation constant Kd was calculated by curve fitting in the same manner as <7> of Example 1. As a result, the dissociation constant Kd is 5.81×10⁻¹⁹ (M), which was exceptional improvement with respect to the natural Kd (1×10⁻⁹ M) of the anti-HER2 nanobody molecule used in the present Example.

### Example 7: Analysis Sensor 10c-P of a Detection Subject that is Particle-Like and Immobilized on a Base Plate (Introducing Anti-HER2 Affibody as a Specific Binding Substance and ATT0647N as a Signal Substance

In the present Example, an analysis sensor 10c-P of a detection subject that is particle-like in the form of being immobilized on a base plate was made. This analysis sensor of a detection subject that is particle-like and immobilized on a base plate set exosome secreted in an SK-BR-3 cell which is an HER2 overexpress-type breast cancer cell line as a detection subject, set HER2 as a target of the detection subject, and selected and introduced an anti-HER2 affibody as a specific binding molecule. In addition, ATT0647N was introduced as a signal substance. In addition, this analysis sensor has an interaction group 25 that interacts with HSA which is a mold. Furthermore, analysis of the detection subject was carried out using the obtained analysis sensor 10c-P of a detection subject that is particle-like and immobilized on a base plate.

Regarding a method of making an analysis sensor 10c-P of a detection subject that is particle-like and immobilized on a base plate, a base plate 10c-P′ for making an analysis sensor of a detection subject that is particle-like in the form of being immobilized on a base plate was made in accordance with FIG. 14-1, FIG. 14-2, FIG. 14-3, FIG. 15 and FIG. 14-4, and furthermore, an analysis sensor 10c-P of a detection subject that is particle-like and immobilized on a base plate was made in accordance with FIG. 14-5.

### <1> Modification of Mold (Step (1-21))

In accordance with FIG. 14-1, a mold HSA modified with a methacryloyl group via a disulfide bond was made.

### <1-1> Reagent

Albumin, from Human Serum (HSA; human serum albumin) (WAKO JUNYAKU KOGYO)
   Mold 60c: HSA
   Binding group BG6″: amino group
   Interaction site 65c: carboxyl group
M27: Compound (5) synthesized in <1> of Example 4
   Binding group BG6′: carboxylic acid-active ester group
   Third reversible linking group 26b: disulfide group
   Polymerizable functional group 27: methacryloyl group

### <1-2> Experimental Procedure

The same procedure as <1-2> of Example 5 was carried out.

### <2> Synthesis of a Molecularly Imprinted Polymer (Step (1-22))

In accordance with FIG. 14-2, a particle of a molecularly imprinted polymer was synthesized.

### <2-1> Reagent and Polymerization Recipe

M25: pyrrolidyl acrylate (PyA)
   Interaction group 25: pyrrolidine group
M28: 2-methacryloyloxyethyl phosphorylcholine (MPC)
M29: N-isopropylacrylamide (NIPAm)

TABLE 7

| Reagent | Amount of use |
|---|---|
| Methacryloyl group modification HSA | 18.3 mg (0.30 μmol) |
| N-Isopropylacrylamide (NIPAm) | 610 mg (5.40 mmol) |
| 2-Methacryloyloxyethyl Phosphorylcholine (MPC) | 88.5 mg (0,30 mmol) |
| Pyrrolidyl acrylate (PyA) | 63.6 mg (0.45 mmol) |
| N,N′-Methylenebisacrylamide (MBAA) | 46.2 mg (0.30 mmol) |
| 2-2′-Azobis(2-methyl propionamide) dihydrochloride (V-50) | 316 mg (1.20 mmol) |
| PBS (10 mM phosphate, 140 mM NaCl, pH7.4) | 50 mL |

### <2-2> Experimental Procedure

Emulsifier-freer precipitation polymerization was carried out for 16 hours at 70° C. in a nitrogen atmosphere in a 50 mL Schlenk flask in accordance with the polymerization recipe of Table 7 to synthesize particle MIP-NGs of a molecularly imprinted polymer 20c.

### <3> Removal of Mold (Step (1-23))

In accordance with FIG. 14-3, the mold was removed from the molecularly imprinted polymer 20c. The specific procedure is the same as <3> of Example 5.

### <4> Particle Analysis

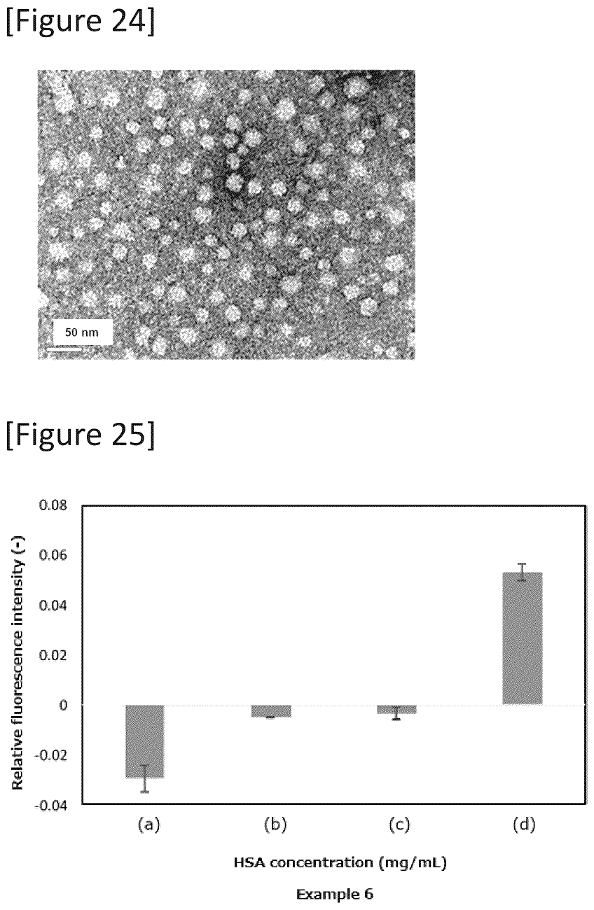

Regarding the obtained particle MIP-NGs of the molecularly imprinted polymer 20c, dynamic light scattering (DLS) measurement and transmission electron microscope (TEM) measurement were carried out in the same manner as <5> of Example 4. The measurement results are shown in the table below. In addition, the TEM image is shown in FIG. 24. In FIG. 24, the length of the scale bar is 50 nm.

TABLE 8

| DLS | | | | TEM |
|---|---|---|---|---|
| | Volume | | | Number |
| Z average | average | | Solid | average |
| particle | particle | | component | particle |
| size | size | PDI | concentration | size |
| 152.5 nm | 27.96 nm | 0.31 | 0.04 | 20-30 nm |

### <5> Fixing of a Particle to a Base Plate

In accordance with FIG. 15, the particle MIP-NGs of the molecularly imprinted polymer 20c obtained in <3> above was immobilized on the base plate. The specific Experimental procedure is the same as <6> of Example 4 and <4> of Example 5. This caused obtainment of a base plate 10c-P′ for making an analysis sensor of a detection subject that is particle-like and immobilized on the base plate.

### <6> Introduction of the Functional Group 24b (Step (1-24))

In accordance with FIG. 14-4, the functional molecule R24 was introduced to the binding group 26b″ of the molecular imprint recessed part 21c. The specific Experimental procedure is the same as <6> of Example 5.

### <7> Introduction of an Affibody and a Fluorescence Substance (Step (2) and Step (3))

In accordance with FIG. 14-5, an anti-Her2 affibody and a fluorescence substance (Atto647N-NHS) were introduced to the base material 10c-P′ for making a sensor for analyzing a detection subject that is immobilized on the base plate. The specific Experimental procedure is the same as <7> of Example 5. This caused obtainment of an analysis sensor 10c-P of a detection subject that is particle-like and immobilized on the base plate.

### <8> Analysis of a Detection Substance by Fluorescence Measurement

Exosome capturing test using a fluorescence microscope with a SIC automatic dispensing apparatus was carried out using the sensor 10c-P for analyzing a detection subject that is immobilized on a base plate obtained in <7> above.

### <8-1> Experimental Procedure

Sample liquids (a) to (d) comprising exosome secreted by an SK-BR-3 cell and HSA at the concentrations shown in the Table below were prepared.

TABLE 9

| | SK-BR-3 cell-driven | |
|---|---|---|
| | exosome final | HSA final |
| | concentration | concentration |
| | (M) | (M) |
| (a) | 1.93 × 10⁻¹⁶ | 0 |
| (b) | 1.93 × 10⁻¹⁶ | 0.72 × 10⁻⁴ |
| (c) | 1.93 × 10⁻¹⁶ | 1.50 × 10⁻⁴ |
| (d) | 1.93 × 10⁻¹⁶ | 7.52 × 10⁻⁴ |

The prepared sample liquids were added to the analysis sensor 10c-P of a detection subject that is particle-like and immobilized on a base plate obtained in <7> above.

Regarding the measurement conditions of the fluorescence microscope, per each sample, the measurement location is point 3 on the base plate, the filter is Cy5, the objective lens is 5-fold, the exposure time is 0.1 second, the power of light is 100%, and the light source is a mercury lamp.

### <8-2> Result

The relative fluorescence intensity was calculated in the same manner as Example 1. The result is shown in FIG. 25. As shown in FIG. 25, regardless of the point that sample liquids (a) to (d) all comprise the same concentration of detection subject (exosome secreted by an SK-BR-3 cell), while no quenching was confirmed when there is a certain degree of concentration of HSA like sample liquid (d), quenching was confirmed when there is zero or low HSA concentration like sample liquids (a) to (c). The result is recognized as achieving the double targeting that while HSA is captured by the interaction group 25 and the molecular imprint recessed part 21c which uses HSA as a mold, whereby an anti-Her2 affibody (specific binding group 42) is shielded, as shown in FIG. 16-1 in sample liquid (d) wherein there is a certain degree of concentration of HSA, HSA is not captured and anti-Her2 affibody (specific binding group 42) is exposed to capture the exosome secreted by an SK-BR-3 cell which is a detection subject as shown in FIG. 16-2 in sample liquids (a) to (c) with zero or low HSA concentration.

Table 10 shows the summary of the sensors for analysis of a detection subject made in Examples 1 to 7 and Comparative Example 2 above.

TABLE 10

| | | | Comparative | | | | | |
|---|---|---|---|---|---|---|---|---|
| | Example 1 | Example 2 | Example 2 | Example 3 | Example 4 | Example 5 | Example 6 | Example 7 |
| Sensor type | 10a₁-C | 10a₁-C | Antibody | 10₁ | 10-PC | 10b-PC | 10a-C | 10c-P |
| | | | substitution type | | | | | |
| | | | of Examples | | | | | |
| | | | 1and 2 | | | | | |
| MIP polymer shape | Film | Film | Film | Film | Particle | Particle | Film | Particle |
| MIP polymer | — | — | — | — | Volume | | — | Volume |
| particle size (nm) | | | | | average: 156.4 | | | average: 27.96 |
| | | | | | Z average: | | | Z average: |
| | | | | | 128.6 | | | 152.5 |
| | | | | | Number | | | Number |
| | | | | | average: 50-80 | | | average: 20-30 |
| Sensor shape | Base plate | Base plate | Base plate | Base plate | Base plate | Base plate | Base plate | Particle |
| Mold | Silica | Silica | Silica | Silica | HSA | HSA | Silica | HSA |
| | particle | particle | particie | particle | | | particle | |
| Mold size (nm) | 200 | 200 | 200 | 200 | — | — | 200 | — |
| Mold molecular | — | — | — | — | 66500 | 66500 | — | 66500 |
| weight | | | | | | | | |
| Signal group 43 | Alexa Flour | Rhodamine | Alexa Flour | Fusion | — | ATTO647N | Alexa Flour | ATTO647N |
| | 647 | | 647 | EGFP | | | 647 | |
| Specific binding | Anti-Her 2 | Anti-Her 2 | Anti-Her 2 | Anti-Her 2 | Anti-Her 2 | Anti-Her 2 | Anti-Her2 | Anti-Her 2 |
| group 42 | affibody | affibody | affibody | affibody | affibody | affibody | nanobody | affibody |
| Different signal | Fusion | Fusion | — | — | — | — | — | — |
| group sg | EGFP | EGFP | | | | | | |
| Specific binding | 6,000 | 6,000 | 150,000 | 6,000 | 6,000 | 6,000 | 15,000 | 6,000 |
| group 42 molecular | | | | | | | | |
| weight | | | | | | | | |
| Specific binding group | 1.06 × 10⁻¹⁰ | 1.06 × 10⁻¹⁰ | 5.8 × 10⁻¹⁰ | 1.06 × 10⁻¹⁰ | 3.2 × 10⁻¹¹ | 3.2 × 10⁻¹¹ | 1 × 10⁻⁹ | — |
| natural dissociation | | | | | | | | |
| constant (M) | | | | | | | | |
| Dissociation constant | 6.1 × 10⁻¹⁸ | 8.41 × 10⁻¹⁸ | 6.34 × 10⁻¹⁶ | 2.29 × 10⁻¹⁷ | 2.0 × 10⁻¹⁸ | 1.0 × 10⁻¹⁷ | 5.81 × 10⁻¹⁹ | |
| by sensor (M) | | | | | | | | |

### Example 8: Immune Response Using a Particle (Non-Immobilized) for Sensing for PSA

In the present example, a particle of which sensing subject is set as porcine serum albumin (PSA) was used in a free state of not being immobilized on a base plate, wherein immune response to each of PSA and transferrin was tested.

The particle with PSA as a sensing subject (fluorescence group introduction MIP-NGs; Example 4) was made with the same method as the “Synthesis of a nanoparticle for sensing-3” and “synthesis of a nanoparticle for sensing-4” of Example 7.

80 μg/mL of fluorescence group introduced MIP-NGs was added with a protein solution (in 10 mM phosphate buffer liquid (pH7.4) comprising 140 mM NaCl) so that the final concentration of the protein (PSA or transferrin) would be 5, 10, 50, 100, 500, 1000 μg/mL (final solution volume: 500 μL), and incubated for 15 minutes at 25° C. Then, the fluorescence spectrum of a solution was measured with a fluorescence spectrophotometer (F-2500, Hitachi High-Tech) (excitation wavelength λex: 647 nm, λem: 658 to 690 nm). The result thereof is shown in FIG. 16. In addition, the relative fluorescence intensity of the fluorescence wavelength 557 nm with respect to the protein concentration of each of PSA and transferrin (PSA: 5, 10, 50, 100, 500, 1000 μg/mL, transferrin: 10, 50, 100, 500, 1000 μg/mL) of PSA and transferrin was plotted. The result thereof is shown in FIG. 17.

As shown in FIG. 16, when a PSA solution is added to the fluorescence group introduction MIP-NGs of Example 4 in a free state, increase in fluorescence intensity was observed as the concentration increased. Since the same phenomenon is confirmed (Example 7) in the case of fixing the fluorescence group introduction MIP-NGs of Example 4 to a base plate, the increase in fluorescence intensity shown in FIG. 16 was suggested to be fluorescence intensity change associated with the PSA binding to the fluorescence group introduction MIP-NGs of Example 4. In addition, as shown in FIG. 17, greater fluorescence intensity change was shown in the case of adding PSA compared to the fluorescence change of the case of adding transferrin which is a reference protein. This showed that the fluorescence group introduction MIP-NGs of Example 4 can selectively adsorb PSA and transmit the binding information as a fluorescence signal even in a free state of not being immobilized on a base plate.

### <Example 9: Synthesis of a Nanoparticle for Sensing-5>

In the present Example, a nanoparticle for Fc domain sensing (and for IgG sensing via Fc domain capturing), which corresponds to the nanoparticle 10a for sensing of FIG. 2, was synthesized.

### 9-1. Synthesis of a Functional Monomer

The functional monomer shown in Formula 51b was designed and synthesized in accordance with the following scheme. Furthermore, the functional monomer shown in Formula 51b has a phenylboronic acid group and a secondary amino group, wherein the bornyl aryl group interacts with a sugar chain of an Fc domain of IgG and the secondary amino group is used for introduction of a fluorescence molecule in post-imprinting modification in MIP-NGs.

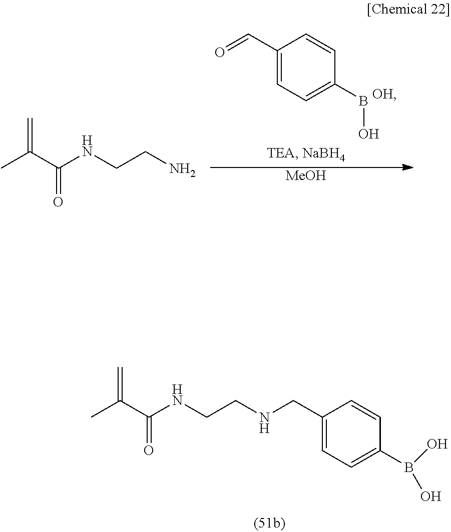

Ethylenediaminemonomethacrylamide hydrochloride (ca. 1.57 mmol) and 4-formylphenylboronic acid (215 mg, 1.5 mmol) and triethylamine (210 μL, 1.5 mmol) were dissolved in methanol (10 mL) to be stirred at room temperature (0.5 h). Reaction tracking was carried out at TLC (MeOH→UV@254 nm and ninhydrin), and appearance of a spot (Rf: 0.55, purple) dyed by ninhydrin and UV absorption was confirmed, from which it was judged that a Schiff base is formed, to which NaBH₄ (120 mg, 9.0 mmol) was added. After the addition, stirring was further carried out overnight (8 h) at room temperature. Since the targeted object spot (Rf: 0.25, yellow) was confirmed at TLC (MeOH UV@254 nm and ninhydrin), the reaction was terminated and the solvent underwent vacuum distillation with a rotary evaporator. The residue was purified using an automated column chromatography system (silica: universal premium, MeOH/DCM=70/30→100/0, GR) to obtain a targeted object.

Yield: 175 mg (0.668 mmol, 42.5%)

¹H-NMR (500 MHz, D₂O): δ=7.62 (d, 2H, phenyl), 7.30 (d, 2H, phenyl), 5.69 (s, 1H, vinyl), 5.44 (s, 1H, vinyl), 3.97 (s, 2H, N—CH₂—), 3.47 (t, 2H, —CH₂—), 2.99 (br, 2H, —CH₂—), 1.89 (s, 3H, -Me)

### 9-2. Preparation of an Fc Domain

10 mg of IgG was dissolved in 10 mM phosphate buffer saline (pH7.4, 140 mM NaCl) (5 mL) to prepare an antibody solution. 0.02M L-cysteine, 0.002M EDTA, and 0.1 mg/mL papain were dissolved in 10 mM phosphate buffer saline (pH7.4, 140 mM NaCl) to prepare an enzyme solution. The same volume of antibody solution and enzyme solution are mixed to be reacted for 24 hours at 37° C. Then, 10 kDa of ultrafiltration film (7500 g, 20 minutes×3) was used to the reaction liquid to substitute the solvent with 20 mM phosphate buffer (pH7.0). Next, 100 kDa of ultrafiltration film (Amicon Ultra) (7500 g, 20 min×3) was used to separate an unreacted IgG. The obtained solution (1 mL) was purified using a Protein A column (Hitrap™ Protein A HP, Cytiva). 20 mM phosphate buffer (pH7.0) was used as a binding buffer and citrate buffer (pH 3.0) was used as an elution buffer to carry out purification at a syringe (flow rate: about 1 mL/min). Regarding the solution after the purification, 10 kDa of ultrafiltration film (7500 g, 20 minutes×3) was used to substituted the solvent with 10 mM carbonate buffer liquid (pH 9.2). The purification of the Fc domain was confirmed with SDS-PAGE.

### 9-3. Synthesis of a Nanoparticle for Sensing-5

The mixture (pre-polymerization solution) of the components shown in the table below was exposed to emulsifier-free precipitation polymerization for 12 hours at 50° C. to synthesize a polymer nanogel (Step 1). In order to render the particle purification operation easy, labeling monomer was also used as a monomer to be comprised in the pre-polymerization solution. The solution (2 mL) after the polymerization was substituted with 10 mM phosphate buffer liquid (pH7.4, 140 mM NaCl) at an ultrafiltration film (10 kDa, 7500×g, 20 minutes×3). The obtained solution (2 mL) went through size exclusion chromatography (Sephadex G-100) to remove an unreacted monomer. The solution (1 mL) obtained thereby and 40 mg/mL SDS aqueous solution (1 mL) were mixed, incubated for 5 minutes, then added to an anion exchange resin (DEAE-Sephadex, 10 cm, 1.5 cm i.d.) to elute 10 mM Tris-HCl buffer (pH7.4, 140 mM NaCl) as an eluate (Step 2). Then, purification was carried out by going through a desalting column (eluate: 10 mM phosphate buffer (pH7.4, 140 mM NaCl)) in order to remove SDS.

TABLE 11

| | | | Comparative |
|---|---|---|---|
| | | Example 5 | Example 2 |
| | | MIP-NGS | NIP-NGS |
| Mold protein | Fe domain (IgG fragment) | 2.50 mg | — |
| | | (0.050 μmol) | |

| | | |
|---|---|---|
| N-substituted or | N-Isopropylacrylamide (NIPAm) | 102 mg (0.90 mmol) |
| non-substituted | | |
| (meth)acrylamide | | |
| Biocompatible | 2-Metacryloyloxyethyl | 3.69 mg (0.0125 mmol) |
| monomer | phosphorylcholine (MPC) | |
| Crosslinking agent | N,N′-Methylenbisacrylamide (MBAA) | 7.71 mg (0.050 mmol) |
| Labeling monomer | Methacryloxyethyl Thiocarbamoyl | 0.42 mg (0.625 μmol) |
| | Rhodamine B (MTRB) | |
| Functional momoner | Functional monomer shown in Fomrula | 15.73 mg (0.060 mmol) |
| | (51b) | |
| Initiating agent | 2-2′-Azobis(2-methyl propion amide) | 54.2 mg (0.20 mmol) |
| | dihydrochloride (V-50) | |
| Solvent | 10 mM Carbonate buffer 2% DMSO | 25 ml |
| | (pH 9.2) | |

Regarding the obtained MIP-NGs (Example 5) and NIP-NGs (Comparative Example 4), when the particle size was measured in the same manner as Example 1, existence of a nanometer-order particle was confirmed. Specifically, with Z average particle size, the particle size was 21 nm in MIP-NGs (Example 5) and the particle size was 18 nm in NIP-NGs (Comparative Example 4). The Z potential was shown to be positively charged at 19 mV in MIP-NGs (Example 5) and 2.9 mV in NIP-NGs (Comparative Example 4). These charges are considered to be derived from the secondary amino group (and polymerization initiating agent) of the functional monomer. In view of the above, it is suggested that a particle incorporated with a functional monomer has been synthesized. In addition, in view of the result of the fluorescence measurement (λₑₓ=280 nm) of a particle solution before and after the purification, since there was loss of the fluorescence derived from tryptophan comprised in the Fc region near 340 nm where it was recognized before the purification, it was confirmed that washing and removal of most Fc domain fragments were able to be carried out.

Furthermore, 5 μL of ATT0647N NHS (10 mg/mL) DMSO solution was added to MIP-NGs (0.5 mg/mL, 1 mL) after the purification to be incubated for 2 hours at 25° C. (Step 3). Ultrafiltration (10 kDa, 7500×g, 20 minutes×3) of the solution after the reaction removed the unreacted fluorescence molecule to obtain the fluorescence group introduction MIP-NGs (Example 5). Furthermore, fluorescence introduction was confirmed based on the point that there was observation of a fluorescence peak near 670 nm which was not found before the introduction based on the fluorescence measurement (excitation wavelength: 647 nm) before and after the fluorescence molecule (ATT0647N) introduction operation. The same operation was carried out to the NIP-NGs to obtain the fluorescence group introduction NIP-NGs (Comparative Example 4).

### 9-4. Base Plate for Sensing-4

The gold sputter glass base plate (4.3×9.8 mm) was washed with pure water and EtOH, and UV-03 washing (20 minutes) was carried out. The base plate after the washing was immersed in an 1 mM 11-mercaptoundecanoic acid EtOH solution to introduce carboxyl group to the surface utilizing self-assembled monolayer formation (25° C., 24 hour). After washing the base plate with EtOH, the base plate was immersed in an aqueous solution wherein 0.2M EDC and 0.05M NHS were dissolved to carry out activation of the surface carboxyl group (25° C., 1 hour). The solution (in PBS, 0.5 mg/mL) of 50 μL of fluorescence group introduction MIP-NGs (Example 5) or fluorescence group introduction NIP-NGs (Comparative Example 4) was added onto the base plate after the reaction to fix the nanoparticle by amine linking (25° C., 1 hour). Then, 1 M aminoethanol aqueous solution (50 μL) was dropped to the unreacted active ester to carry out inactivation (25° C., 0.5 hour). Finally, Protein free blocking buffer (50 μL) was dropped to carry out blocking of the surface (25° C., 0.5 hour). Furthermore, the florescence intensity at the surface before and after immobilization was measured, wherein since there had been observation of increase in the fluorescence intensity at the base plate after fixing, fixing of a particle was confirmed.

### 9-5. Fluorescence Measurement

Regarding the fluorescence detection capacity of a protein of the base plate for sensing that had been made, examination was carried out using a fluorescence microscope with an automatic liquid handling robot. The measurement sequence conditions are as shown below.

Fluorescence Microscope
Filter: Cy5
Object lens: ×5
Exposure time: 0.1 second
Power of light: 12%
Light source: mercury lamp
ROI: base plate central part 3×5=15 locations

Sequence of Automatic Dispensation Apparatus
1. Chip attachment
2. Sample suction 150 μL
3. Reaction 5 minutes (25° C.)
4. Measurement position keep Repeat 2 to 4

FIG. 18 shows the relative fluorescence intensity change of when an Fc domain fragment (0-1600 nM) which is a mold protein was added to the base plate for sensing that had been made. Since great fluorescence change was shown at the base plate wherein MIP-NGs (Example 5) is immobilized compared to the base plate wherein NIP-NGs (Comparative Example 4) is immobilized, it is suggested that a binding space of the Fc domain fragment was formed by molecular imprinting in MIP-NGs (Example 5), and a functional monomer was incorporated into said space and fluorescence labeling was carried out by post-imprinting modification, which enabled fluorescence signal conversion of the binding of the Fc domain fragment. In addition, the apparent binding constant (Kd) was calculated as 9.8×10⁻⁹ M from the obtained fluorescence change rate.

The Fc domain fragment and the whole IgG and Lysozyme (Lyz) as reference proteins were used to test the protein selectivity of the base plate for sensing that had been made. FIG. 19 shows the fluorescence response upon adding each reference protein (100 nM) with the relative fluorescence intensity change upon adding the Fc domain fragment (100 nM) as 1. MIP-NGs (Example 5) showed the greatest fluorescence change upon adding the Fc domain fragment. In addition, since about 70% of the whole IgG having the Fc domain fragment as the partial structure thereof is also responding, it is suggested that the Fc site of the IgG can be recognized. In other words, it is shown that the MIP-NGs (Example 5) that had been made while using the Fc domain fragment as a mold can also detect IgG via capturing of the Fc domain. Meanwhile, whole IgG and Lyz both showed greater fluorescence response than the Fc domain fragment in the NIP-NGs (Comparative Example 4). It is presumed that this is a non-specific bond with a functional monomer residue that is randomly present on the NIP-NGs (Comparative Example 4) surface and the fluorescence molecule introduced thereto. The pI of the whole IgG is up to 8.5, and Lyz is 11.2, which are positively charged in a neutral solution. In view of the above, it is considered that the electrostatic interaction with the phenylboronic acid group of the functional monomer, hydrogen binding with an amide binding site, and the like contribute to the binding.

While the preferable embodiments of the present disclosure are as discussed above, the present disclosure is not limited thereto, and other various embodiments that do not deviate from the gist of the present disclosure are carried out. The present application claims priority over Japanese Patent Application No. 2020-159008 filed on Sep. 23, 2020 in Japan, and PCTJP2021/007726 filed on Mar. 1, 2021. The entire content thereof is incorporated herein by reference.

### REFERENCE SIGNS LIST

Base material for making a sensor for analysis . . . 10′, 10a′, 10b′, and 10c′
Base material for king a sensor for analysis (base plate-type) . . . 10-C′, and 10a-C′
Base material for making a sensor for analysis (particle-like) . . . 10-P′, 10a-P′, and 10c-P′
Base material for making a sensor for analysis (particle-fixed base plate-type) . . . 10-PC′, and 10b-PC′
Sensor for analysis . . . 10, 101, 10a, 10b, and 10c
Sensor for analysis (base plate-type) . . . 10-C, 10a-C, and 10a₁-C
Sensor for analysis (particle-like) . . . 10-P, 10a-P, and 10c-P
Sensor for analysis (particle-immobilized base plate-type) . . . 10-PC, and 10b-PC
Molecularly imprinted polymer . . . 20, 20a, 20b, and 20c
Molecular imprint recessed part . . . 21, and 21c
Group for binding a specific binding molecule to a target . . . 22′, and 22b′ (thiol, disulfide)
Binding group . . . 22″ (disulfide)
Monomer comprising a group for binding a specific binding molecule and a polymerizable functional group . . . M22
First reversible linking group . . . 22
Group for binding a signal substance . . . 23′, and 23b′
Second reversible linking group . . . 23
Polymerizable monomer (monomer providing a group for binding a signal monomer) . . . M23₁, and M23₂
Functional group . . . 24b
Functional molecule having a functional group and a group for binding a specific binding molecule to a target . . . R24
Interaction group (for a mold of said molecular imprint recessed part of detection subject) . . . 25
Monomer comprising an interaction group for a mold . . . M25
Binding group . . . 26b″
Third reversible linking group 26b
Polymerizable functional group . . . 27
Polymerizable monomer . . . M22₁
Polymerizable monomer (e.g., Compound (5)) . . . M222
Polymerizable monomer (e.g., biocompatible monomer) . . . M28
Polymerizable monomer (e.g., acrylamide monomer) . . . M29
Base plate . . . 30
Binding group (on a base plate) . . . BG1′
Specific binding group (for a target) . . . 42
Specific binding molecule (for a target) . . . R42
Different signal group . . . sg
Specific binding group having a different signal group . . . 42₁
Signal group . . . 43
Signal substance . . . R43
Specific binding group having a signal group . . . 44
Mold . . . 60, 602, 602, and 60c
Interaction site (on a mold) . . . 65c
Binding group (on a mold) . . . BG6″, BG9″BG22″, and BG23″
Binding group (corresponding to BG6″, BG9″, BG22″, and BG23″) . . . BG6′, BG9′BG22′, and BG23′
Detection subject . . . 70
Target . . . 72
INI . . . Polymerizable initiating group
SAM . . . Monolayer

1. A base material for use in making a sensor for use in analyzing a detection subject, comprising a molecularly imprinted polymer having:
a molecular imprint recessed part that receives a target of a detection subject; and
a group for use in binding a binding molecule to the target provided within the molecular imprint recessed part,
wherein the group for use in binding a binding molecule is a group adapted to a small substance.

2. A base material for use in making a sensor for use in analyzing a detection subject, comprising a molecularly imprinted polymer having:
a molecular imprint recessed part that receives a target of a detection subject; and
a group for use in binding a binding molecule to the target provided within the molecular imprint recessed part,
wherein the molecular imprint recessed part has a shape or configuration adapted to a small substance.

3. The base material for use in making a sensor for use in analyzing a detection subject of claim 1 or 2, further having a group for use in binding a signal substance within the molecular imprint recessed part.

4. The base material for use in making a sensor for use in analyzing a detection subject of claim 3, wherein the group for use in binding a binding molecule and the group for use in binding a signal substance are independent from one another in the molecular imprint recessed part surface.

5. The base material for use in making a sensor for use in analyzing a detection subject of claim 3, wherein the group for use in binding a binding molecule and the group for use in binding a signal substance configure the functional group shown in Formula (1) below within the molecular imprint recessed part.
[Chemical 23]
—R²-L¹-R¹ (1)
<R¹ represents the group for use in binding a binding molecule, L¹ represents a direct binding or linking group, and R² represents a linking group comprising the group for use in binding a signal substance.>

6. The base material for use in making a sensor for use in analyzing a detection subject of claim 5, wherein a linking group comprising the group for use in binding a signal substance is represented by Formulas (2) and (3) below. 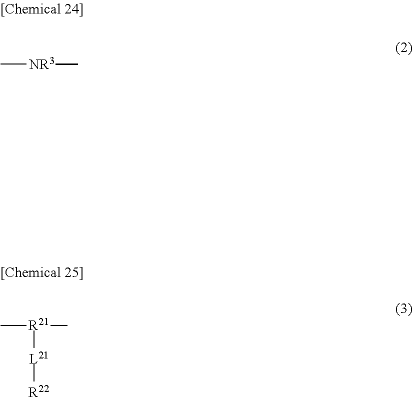
<R³ represents an alkyl group with a carbon number of 1 to 8 that may comprise hydrogen or a polyalkylene glycol chain, R²¹ represents a nitrogen atom or hydrocarbon, L²¹ represents a direct binding or linking group, and R²² represents the group for use in binding a signal substance.>

7. The base material for use in making a sensor for use in analyzing a detection subject of any of claims 1 to 6, wherein the group for use in binding a binding molecule is a group selected from the group consisting of an amino group, a carboxyl group, a boronic acid group, a cis-diol group, an aldehyde group, a ketone group, an oxyamino group, a biotin (biocytin, desthiobiotin) group, a nickel-nitrilotriacetic acid-driven group, a thiol group, and a pyridyl disulfide group.

8. The base material for use in making a sensor for use in analyzing a detection subject of any of claims 1 to 7, wherein the group for use in binding a binding molecule is a group selected from the group consisting of a nickel-nitrilotriacetic acid-driven group, a thiol group, and a pyridyl disulfide group.

9. The base material for use in making a sensor for use in analyzing a detection subject of any of claims 1 to 8, wherein the group for use in binding a binding molecule is adapted to a small substance via an Fc domain binding protein of an antibody, avidin, streptavidin, NeutrAvidin, a polymer comprising one or more of the binding group of claim 7, dendrimer, or oligoDNA.

10. The base material for use in making a sensor for use in analyzing a detection subject of any of claims 1 to 9, wherein the Fc domain binding protein of an antibody is protein A, protein G, protein L, or protein A/G.

11. The base material for use in making a sensor for use in analyzing a detection subject of any of claims 1 to 10, having an interaction group for a mold of the molecular imprint recessed part or the detection subject within the molecular imprint recessed part.

12. The base material for use in making a sensor for use in analyzing a detection subject of any of claims 1 to 11, wherein the binding molecule is a specific binding molecule.

13. The base material for use in making a sensor for use in analyzing a detection subject of any of claims 1 to 12, wherein the specific binding molecule is selected from the group consisting of an antibody mimetic, a phospholipid binding protein, an artificial (polymer) receptor, and lectin.

14. The base material for use in making a sensor for use in analyzing a detection subject of any of claims 1 to 13, wherein the detection subject is a microparticle having a film structure.

15. The base material for use in making a sensor for use in analyzing a detection subject of any of claims 1 to 14, wherein a mold of the molecular imprint recessed part is a particle core or a biomolecule.

16. The base material for use in making a sensor for use in analyzing a detection subject of any of claims 1 to 15, wherein a mold of the molecular imprint recessed part is a silica particle or serum protein.

17. The base material for use in making a sensor for use in analyzing a detection subject of any of claims 1 to 16, wherein a mold of the molecular imprint recessed part is a serum protein, the detection subject is a microparticle having a film structure, and the target is a molecule expressed on a surface of the microparticle.

18. The base material for use in making a sensor for use in analyzing a detection subject of any of claims 1 to 17, comprising a base plate and a polymer film provided on a surface of the base plate,
wherein the imprint polymer configures the polymer.

19. The base material for use in making a sensor for use in analyzing a detection subject of any of claims 1 to 17, wherein the imprint polymer is particle-like.

20. The base plate for use in making a sensor for use in analyzing a detection subject of claim 19, wherein the imprint polymer that is particle-like is immobilized on a base plate.

21. A sensor for use in analyzing a detection subject, comprising:
the base material for use in making a sensor for use in analyzing a detection subject of any one of claims 1 to 20; and
a specific binding group for a target of the detection subject bound to the group for use in binding a specific binding molecule,
wherein the specific binding molecule is a small substance group.

22. The sensor for use in analyzing a detection subject of claim 21, further comprising a signal group bound to the group for use in binding a signal substance.

23. An analysis method of a detection subject, comprising:
the step of contacting a sample comprising a detection subject to the sensor for use in analyzing a detection subject of claim 21 or 22 and binding the detection subject to the specific binding group; and
the step of detecting a bong between the specific binding group and the detection subject.

24. A method of manufacturing a base material for use in making a sensor for use in analyzing a detection subject, comprising:
the monolayer formation step (1-1) of forming a monolayer having a group for use in binding a binding molecule to a target of a detection subject and a polymerization initiating group on a surface at a base plate;
the step (1-2) of introducing a mold to the group for use in binding a binding molecule via a first reversible linking group;
the step (1-3) of adding a polymerizable monomer, using the polymerizable monomer as a substrate, using the polymerization initiating group as a polymerizable initiating agent, and synthesizing a molecularly imprinted polymer for a part of a surface of the mold to form a polymer film on the base plate surface; and
the step (1-4) of cleaving the first reversible linking group for conversion to the group for use in binding a binding molecule as well as removal of the mold,
wherein the group for use in binding a binding molecule is a group adapted to a small substance.

25. A method of manufacturing a base material for use in making a sensor for use in analyzing a detection subject, comprising:
the monolayer formation step (1-1) of forming a monolayer having a group for use in binding a binding molecule to a target of a detection subject and a polymerization initiating group on a surface at a base plate;
the step (1-2) of introducing a mold to the group for use in binding a binding molecule via a first reversible linking group;
the step (1-3) of adding a polymerizable monomer, using the polymerizable monomer as a substrate, using the polymerization initiating group as a polymerizable initiating agent, and synthesizing a molecularly imprinted polymer for a part of a surface of the mold to form a polymer film on the base plate surface; and
the step (1-4) of cleaving the first reversible linking group for conversion to the group for use in binding a binding molecule as well as removal of the mold,
wherein the molecular imprint recessed part has a shape or configuration adapted to a small substance.

26. A method of manufacturing the base material for use in making a sensor for use in analyzing a detection subject of claim 16, comprising:
the modification step of modifying the surface of the mold via a polymerizable functional group via a second reversible linking group,
wherein the polymerizable monomer and the polymerizable functional group are used as a substrate and the polymerization initiating group is used as a polymerizable initiating agent to synthesize a molecularly imprinted polymer for a part of the surface of the mold in the step (1-3), and
wherein the first reversible linking group and the second reversible linking group are cleaved for conversion to the group for use in binding a binding molecule and a group for use in binding a signal substance, respectively, as well as removal of the mold in the step (1-4).

27. A method of manufacturing a base material for use in making a sensor for use in analyzing a detection subject, comprising:
the step (1-11) of preparing a mold of a molecular imprint wherein a surface is modified with a polymerizable functional group via a first reversible linking group;
the step (1-12) of using the polymerizable functional group as a substrate and synthesizing a molecularly imprinted polymer for a part of the surface of the mold; and
the step (1-13) of cleaving the first reversible linking group and removing the mold from the molecularly imprinted polymer to generate the group for use in binding a binding molecule,
wherein the group for use in binding a binding molecule is adapted to a small substance.

28. A method of manufacturing a base material for use in making a sensor for use in analyzing a detection subject, comprising:
the step (1-11) of preparing a mold of a molecular imprint wherein a surface is modified with a polymerizable functional group via a first reversible linking group;
the step (1-12) of using the polymerizable functional group as a substrate and synthesizing a molecularly imprinted polymer for a part of the surface of the mold; and
the step (1-13) of cleaving the first reversible linking group and removing the mold from the molecularly imprinted polymer to generate the group for use in binding a binding molecule,
wherein the molecular imprint recessed part has a shape or configuration adapted to a small substance.

29. A method of manufacturing a base material for use in making a sensor for use in analyzing a detection subject, comprising:
the step (1-21) of preparing a mold of a molecular imprint wherein a surface is modified with a polymerizable functional group via a third reversible linking group;
the step (1-22) of using the polymerizable functional group as a substrate and synthesizing a molecularly imprinted polymer for a part of the surface of the mold;
the step (1-23) of cleaving the third reversible linking group and removing the mold from the molecularly imprinted polymer; and
the step (1-24) of introducing a molecule having functional group comprising a group for use in binding a binding molecule for a target of a detection subject and a group for use in binding a signal substance via third reversible linking group,
wherein the group for use in binding a binding molecule is a group adapted to a small substance.

30. A method of manufacturing a base material for use in making a sensor for use in analyzing a detection subject, comprising:
the step (1-21) of preparing a mold of a molecular imprint wherein a surface is modified with a polymerizable functional group via a third reversible linking group;
the step (1-22) of using the polymerizable functional group as a substrate and synthesizing a molecularly imprinted polymer for a part of the surface of the mold;
the step (1-23) of cleaving the third reversible linking group and removing the mold from the molecularly imprinted polymer; and
the step (1-24) of introducing a molecule having functional group comprising a group for use in binding a binding molecule for a target of a detection subject and a group for use in binding a signal substance via third reversible linking group,
wherein the molecular imprint recessed part has a shape or configuration adapted to a small substance.

31. The method of manufacturing a base material for use in making a sensor for use in analyzing a detection subject of claim 29 or 30, further using a monomer comprising an interaction group for the mold as the substrate in the step (1-22).

32. A method of manufacturing the base material for use in making a sensor for use in analyzing a detection subject of any of claims 17 to 21, wherein the binding molecule is a specific binding molecule.

33. A method of manufacturing a sensor for use in analyzing a detection subject, comprising:
the step (1) of carrying out the method of manufacturing a base material for use in making a sensor for use in analyzing a detection subject of any of claims 24 to 32; and
the step (2) of binding the binding molecule to the group for use in binding a binding molecule.

34. A method of manufacturing a sensor for use in analyzing a detection subject, comprising:
the step (1) of carrying out the method of manufacturing a base material for use in making a sensor for use in analyzing a detection subject of any of claims 26, 29 and 31;
the step (2) of binding the binding molecule to the group for use in binding a binding molecule; and
the step (3) of binding a signal substance to the group for use in binding a signal substance.
